(19) United States
(12) Patent Application Publication
(10) Pub. No.: US 2011/0182904 A1
(43) Pub. Date: Jul. 28, 2011

(54) ANTIBODIES TO BONE MORPHOGENIC PROTEINS AND RECEPTORS THEREFOR AND METHODS FOR THEIR USE

(76) Inventors: Deborah Zimmerman, Oakland, CA (US); Mark Selby, San Francisco, CA (US); Mohan Srinivasan, San Jose, CA (US); Alasdair Bell, Mountain View, CA (US); Sujata Singh, Saratoga, CA (US); Richard Theolis, Santa Cruz, CA (US); Heidi N. Leblanc, Mountain View, CA (US); Kyra D. Zens, San Mateo, CA (US); Timothy William Sproul, Livermore, CA (US)

(21) Appl. No.: 12/440,000

(22) PCT Filed: Sep. 5, 2007
(86) PCT No.: PCT/US07/19652

§ 371 Date: Jun. 8, 2010

Related U.S. Application Data
(60) Provisional application No. 60/824,596, filed on Sep. 5, 2006.

(51) Int. Cl. A61K 39/395; C07K 16/22; A61P 19/08; A61P 35/00; C12N 15/13; C12N 15/63; C12N 5/00; C12P 21/08

(52) U.S. Cl. 424/145.1; 530/388.23; 530/387.3; 530/391.1; 530/391.7; 530/391.3; 536/23.53; 435/320.1; 435/325; 435/69.6; 800/4

(57) ABSTRACT

The present invention provides isolated monoclonal antibodies, particularly human monoclonal antibodies, which specifically bind to BMP2, BMP4, BMPR1A, BMPR1B, ACTR1, and/or BMPR2 with high affinity. Nucleic acid molecules encoding the antibodies of the invention, expression vectors, host cells and methods for expressing the antibodies of the invention are also provided. Also provided are immunoconjugates, bispecific molecules and pharmaceutical compositions comprising the antibodies of the invention and, optionally, one or more additional therapeutic. The invention also provides methods for treating diseases associated with abnormal bone formation and ossification mediated by BMP2, BMP4, BMPR1A, BMPR1B, ACTR15 and/or BMPR2.

### CROSS-REFERENCE TO RELATED APPLICATIONS

This application claims priority to U.S. Provisional Application Ser. No. 60/824,596, filed Sep. 5, 2006, which is incorporated by reference herein in its entirety.

### FIELD OF THE INVENTION

The present invention relates generally to the fields of immunology and molecular biology. More specifically, provided herein are antibodies and other therapeutic proteins directed against bone morphogenic proteins (BMPs) and receptors therefor, nucleic acids encoding such antibodies and therapeutic proteins, methods for preparing inventive monoclonal antibodies and other therapeutic proteins, and methods for the treatment of diseases, such as bone diseases and cancers mediated by BMP expression/activity and/or associated with abnormal expression/activity of a receptor therefor.

### BACKGROUND OF THE INVENTION

The human skeleton comprises in excess of 200 articulated bones. During embryogenesis, the skeleton develops from undifferentiated mesenchyme according to a genetic program that dictates temporal and spatial formation. In healthy individuals, postnatal development includes the initiation of new skeletal elements through bone regeneration at sites of bone fracture.

Alteration in the normal regulation of skeletogenesis can result in the abnormal formation of bone in soft-tissues. Shafritz et al., N. Engl. J. Med. 335:555-561 (1996) and Kaplan et al., J. Am. Acad. Orthop. Surg. 2:288-296 (1994). In extreme cases, such abnormal bone formation, also referred to as heterotopic ossification, can lead to clinically significant or devastating consequences, which can dramatically compromise a patient's quality of life. The causes of heterotopic ossification are varied and may be acquired through injury of the central nervous system or soft tissue; vascular disease (e.g., atherosclerosis and valvular heart disease); and arthropathies (e.g., ankylosing spondylitis, psoriatic arthritis, seronegative arthropathies, and diffuse idiopathic skeletal hyperostosis). In other instances, heterotopic ossification may develop through a genetic cause such as fibrodysplasia ossificans progressiva or progressive osseous heteroplasia. Reviewed by Kaplan et al., “Heterotopic Ossification” J. Amer. Acad. of Orth. Surg. 12(2):116-125 (2004).

Spondyloarthritides (SpA) refers to a group of diseases that, together, are characterized by spinal inflammation, significant pain, and functional disability; these diseases greatly impact a patient's quality of life. Braun et al., Arthritis Rheum. 41:58-67 (1998); Zink et al., J. Rheumatol. 27:613-622 (2000); and Dagfinrud et al., Ann. Rheum. Dis. 63:1605-1610 (2004). SpA includes, for example, such debilitating disorders as ankylosing spondylitis, psoriatic spondyloarthritides, reactive spondyloarthritides, spondyloarthritides associated with inflammatory bowel disease, and undifferentiated spondyloarthritides.

Ankylosing spondylitis (AS) and related spondyloarthropathies are among the most common inflammatory rheumatic diseases. In the United States and Northern Europe, these disorders have an estimated prevalence of approximately 0.1% to 0.3%—primarily affecting individuals between 20 and 40 years of age. Khan, “A Worldwide Overview: The Epidemiology of HLA-B27 and Associated Spondyloarthritides,” (Oxford: Oxford University Press (1998)) and Saraux et al., J. Rheumatol. 26:2622-2627 (1999). The characteristic clinical features of AS include inflammatory back pain, usually caused by sacroiliitis and enthesitis. AS typically involves the axial skeleton, but may also affect the peripheral joints (shoulders and hips) and extra-articular structures.

Patients with ankylosing spondylitis present with the most severe spinal involvement due to new bone formation leading to syndesmophytes and ankylosis. AS is thus one of multiple diseases that present with heterotopic ossification. Gladman et al., Arthritis Rheum. 50:24-35 (2004) and Edmunds et al., J. Rheumatol. 18:696-698 (1991). Increasing evidence suggests that in AS an anatomical zone referred to as the enthesis, where tendons and ligaments attach to underlying bone, is the primary target of the pathological process. Ball, Ann. Rheum. Dis. 30:213-223 (1971) and Benjamin and McGonagle, J. Anat. 199:503-526 (2001).

Animal model systems for ankylosing spondylitis and related spondyloarthropathies have been described, most of which are based upon the close association between AS and human leukocyte antigen-B27 (HLA-B27) expression. Reviewed in, Zhang et al., Current Rheum. Reports 4:507-512 (2002). Introduction of the HLA-B27 transgene into rats induces the spontaneous development of a multisystem disorder that involves spondylitis. Hammer et al., Cell 63:1099-1112 (1990). HLA-B27 transgenic mice (C57BL/10) develop peripheral arthritis with progressive stiffening of the ankle or tarsal joints, although the spine is not affected. Weinreich et al., Hum. Immunol. 42:103-115 (1995). It has also been reported that immunity to either of the G1 domains of the proteoglycans aggrecan and versican can induce in BALB/c mice an AS-like pathology, which includes spondylitis, sacroiliitis, and enthesitis. Giant et al., Arthritis Rheum. 30:201-212 (1987) and Shi et al., Arthritis Rheum. 44:S240 (2001). DBA/1 mice are a spontaneous model of arthritis, ankylosing enthesitis and abnormal bone formation. Lories et al., J Clin. Invest. 115(6):1571-9 (2005). Mice deficient in matrix GLA protein have been shown to exhibit spontaneous calcification of arteries and cartilage and, therefore, are used as a model system for vascular calcification. Luo et al., Nature 386:78-81 (1997).

Bone morphogenic proteins (BMPs) are multi-functional growth factors that are members of the transforming growth factor β (TGFβ) superfamily. BMP signaling plays a role in heart, neural, and cartilage development as well as in postnatal bone formation. BMPs ectopically induce a cascade of endochondral bone formation and play a critical role in skeletal and joint morphogenesis. Urist, Science 150:893-899 (1965); Olsen et al., Annu. Rev. Cell Dev. Bial. 16:191-220 (2000); Kronenberg, Nature 423:332-336 (2003); Thomas et al., Nat. Genet. 12:315-317 (1996); Thomas et al., Nat. Genet. 17:58-64 (1997); Polinkowsky et al., Nat. Genet. 17:18-19 (1997); and Storm et al., Nature 368:639-643 (1994).

Approximately 20 members of the BMP family have been identified. BMPs signal through serine/threonine kinase receptors, which include both types I and II. Three type I receptors bind BMP ligands (type IA and IB BMP receptors and type I activin receptor (ActRI). Koenig et al., Mol. Cell. Biol. 14:5961-5974 (1994) and Ten Dijke et al., J. Biol Chem. 269:16985-16988 (1994); and Macias-Silva et al., J. Biol. Chem. 273:25628-25636 (1998). BMPs are synthesized and folded as large dimeric pro-proteins in the cytoplasm and cleaved by proteases during secretion. Each monomer contains about 300 amino acids as the proprotein. The functional carboxy region (100-120 amino acids in each monomer) is released into the extracellular compartment to bind membrane receptors on target cells. Although dimerization of BMPs relies on several disulfide bonds between the two subunits, the precise biochemistry of dimerization and cleavage remains to be characterized. Additionally, there appear to be an array of extracellular proteins that antagonize or otherwise alter the function of BMPs; these proteins include Glypican-3, Noggin, Chordin, Cerberus, and Follistatin. Fainsod et al., Mech. Dev. 63:39-50 (1997); Grisaru et al., Dev. Biol. 231:31-46 (2001); Holley et al., Cell 86:607-617 (1996); Iemura et al., Proc. Natl. Acad. U.S.A. 95:9337-9342 (1998); Jackson at al., Development 124:4113-4120 (1997); Paine-Saunders et al., Dev. Biol. 225:179-187 (2000); Piccolo et al., Cell 86:589-598 (1996); Re'em-Kalma at al., Proc. Natl. Acad. Sci. U.S.A. 92:12141-12145 (1995); Sasai et al., Nature 376:333-336 (1995); and Zimmerman at al., Cell 86:599-606 (1996). Three type II receptors for BMPs have also been identified (i.e. BMPRI, ActRII and ActRIIB). Yamashita at al., J. Cell. Biol. 130:217-226 (1995); Rosenzweig et al., Proc. Natl. Acad. Sci. U.S.A. 92:7632-7636 (1995); Kawabata at al., J. Biol. Chem. 270:5625-5630 (1995).

The type I and II BMP receptors are differentially expressed in various tissues yet both are indispensable for signal transduction. Upon ligand binding, the type I and II BMP receptors form heterotetrameric-activated receptor complexes, which include two pairs of a type I and II receptor complexes. Moustakas and Heidi, Genes Dev. 16:67-87 (2002). Both receptor types are essential for signal transduction. Hogan, Genes Dev. 10:1580-1594 (1996); Nellen et al., Cell 78:225-237 (1994); Ruberte et al., Cell 80:889-897 (1995); ten Dijke et al., Curr. Opin. Cell Biol. 8:139-145 (1996); Weis-Garcia and Massague, EMBO J. 15:276-289 (1996); and Wrana et al., Nature 370:341-347 (1994). Type II receptors have constitutively active kinase activity that phosphorylates type I receptors upon ligand binding. Phosphorylated type I receptors transduce the signal to downstream target proteins.

The type I BMP receptors signal through the Smad proteins (smad 1/5), which are important in relaying the BMP signal from the receptor to the target genes in the nucleus. Upon release from the receptor, the phosphorylated Smad proteins associate with the related protein Smad4, which acts as a shared partner. This complex translocates into the nucleus and participates in gene transcription with other transcription factors.

BMP signaling is controlled at many levels, including via extracellular antagonists such as noggin. Massague, Nat. Rev. Mol. Cell. Biol. 1:169-178 (2000). It has been suggested that untimely or unwanted activation of signaling cascades fundamental for normal development may promote disease processes such as spondyloarthropathies. The effects of BMP signaling on initiation and progression of arthritis by gene transfer of noggin have been described. Lories et al., J. Clin. Invest. 115(6):1571-1579 (2005).

The physiological roles of BMPs and BMP receptor signaling in normal bone formation, including skeletal and limb development, have been studied and recently reviewed in Zhao, Genetics 35:43-56 (2003). During endochondral ossification, mesenchymal cells condense and differentiate into chondrocytes. The chondrocytes undergo a highly organized differentiation program, forming the template for bone formation. Kronenberg, Nature 423:332-336 (2003) and Olsen et al., Annu. Rev. Cell. Dev. Biol. 16:191-220 (2000). BMPs were identified by their ability to promote ectopic cartilage and bone formation. Wozney, Prog. Growth Factor Res. 1:267-280 (1989). The differential affinities of distinct BMP ligands for the three type I receptors, BMPR1A, BMPR1B, and ActR1 (activin receptor type I), contribute to diversity of signaling during the course of development. These receptors participate in chondrogenesis—each having a distinct tissue distribution and function.

Mice deficient for BMP2 and BMP4 are nonviable. Homozygous BMP2 mutant embryos die between embryonic day 7.5 and 10.5 and have defects in cardiac development. Zhang and Bradley, Development 122:2977-2986 (1996). Homozygous BMP4 mutant embryos die between embryonic day 6.5 and 9.5 and are defective in mesodermal differentiation. Winnier et al., Genes Dev. 9:2105-2116 (1995).

Yoon et al. described the generation of mice that are null for both Bmpr1a and Bmpr1b in chondrocytes. Proc. Natl. Acad. Sci. U.S.A. 102(14):5062-5067 (2005). These authors demonstrate that Bmpr1a conditional knockout mice, like Bmpr1b null mice, exhibit few skeletal defects. Mice harboring both mutations, however, develop a severe and generalized chondrodysplasia. These data suggest that overlapping functions for BMPR1A and BMPR1B during early chondrogenesis and that BMP signaling is required for chondrocyte proliferation, survival, and differentiation. Null mutation of the BMPR1A gene causes embryonic lethality in mice; animals die at embryonic day 9.5. Homozygous mutants with morphological defects are detectable at embronic day 7.5 and the embryos are defective in mesoderm formation. Mishina et al., Genes Dev. 9:3027-3037 (1995).

Mice lacking BMPR1B are viable but exhibit defects in the appendicular skeleton. In BMPR1B deficient mice, proliferation of prechondorgenic cells and chondrocyte differentiation in the phalangeal region are reduced. In adult mutant mice, the proximal interphalangeal joint is absent, and the phalanges are replaced by a single rudimentary element, while the distal phalanges are unaffected. The lengths of the radius, ulna, and, tibia are normal, but the metacarpals and metatarsals are reduced. Yi et al., Development 127:621-630 (2000). It has been suggested that BMPR1B likely plays a non-redundant role in cartilage formation in vivo. Gannon et al., Hum. Pathol. 28:339-343 (1997). BMP ligands may utilize multiple type I BMP receptors to mediate their signaling during cartilage and bone formation and that BMPR1B and ActR1A (Alk2) may play synergistic and/or overlapping roles in cartilage and bone formation in vivo. Macias-Silva et al., J. Biol. Chem. 273:25628-25636 (1998).

Noggin is a secreted polypeptide that binds to and inactivates BMP-2 and BMP4. Co-crystal structures of noggin and BMPs show that noggin inhibits BMP signaling by blocking the molecular interfaces of the binding epitopes for both type I and type II BMP receptors. A transgenic mouse model has been established using the osteocalcin promoter to drive the noggin transgene. These animals developed osteoporosis as evidenced by significant reductions in bone mineral density, bone volume, and bone formation rates. Devlin et al., Endocrinology 144:1972-1978 (2003) and Wu el al., J. Clin. Investig. 112:924-924 (2003). In total, these experiments with BMP antagonists demonstrate that regulation of BMP signaling proteins is central to bone formation in vivo.

Animal model systems have been described and used for evaluating the capacity of BMP2 to heal bone defects by initiating chondrogenesis and bone formation. The osteoinductive capacity of BMP2 is consistent with the healing effects on long bone mediated by this growth factor seen in rats, rabbits, dogs, sheep, and non-human primates. Murakami et al., J. Biomed. Mater. Res. 62:169-174 (2002). Injection of BMP-2 locally over the surface of calvariae of mice induced periosteal bone formation on the surface of calvariae without a prior cartilage phase. Chen et al., Calcif. Tissue Int. 60:283-290 (1997). Also, systemic administration of recombinant human BMP2 increases mesenchymal stem cell activity and reverses ovariectomy-induced and age-related bone loss in mouse models suggesting that BMP2 may be effective therapeutically in the treatment of osteoporosis. Turgeman et al., J. Cell. Biochem. 86:461-474 (2002).

Over-expression of BMP2 and 4 as well as BMPR1A is associated with malignancy of the oral epithelium while overexpression of BMP2 has been reported in prostate cancer cells. Jin et al., Oral Oncol. 37:225-233 (2001) and Harris et al., Prostate 24 :204-211 (1994), respectively. BMP has also been shown to promote metastatic behavior in melanoma cell lines. Rothhammer et al., Cancer Res. 65(2):448-56 (2005).

Fibrodysplasia ossificans progressiva (FOP) is a rare and disabling genetic disorder characterized by congenital malformations of the great toes and by progressive heterotopic endochodral ossification in predictable anatomical patterns. Ectopic expression of BMP4 has been found in FOP patients. Gannon et al., Hum. Pathol. 28:339-343 (1997) and Xu et al., Clin. Genet. 58:291-298 (2000). It has recently been shown that patients with FOP have activating mutations in the BMP type I receptor ACVRI. Shore et al., Nat. Gen. 23 April advance online publication (2006). Transgenic mice overexpressing BMP4 under control of a neuron-specific enolase (NSE) promoter have also been described as developing an FOP-like phenotype. Kan et al., Am. J. of Path. 165(41:1107-1115 (2004). Mating these animals with transgenic mice that overexpress noggin prevents the disorder, thus confirming the role of BMP4 in the pathogeneissis of the disease.

SpA is another medical condition with involvement of heterotopic, or abnormal, bone formation. Existing therapeutic modalities for SpA, in particular ankylosing spondylitis, are reviewed in Zochling et al., Curr. Opin Rheumatol. 17:418-425 (2005) and van der Heijde et al., Ann. Rheum. Dis. 61:24-32 (2002). Baseline therapy includes the use of nonsteroidal anti-inflammatory drugs (NSAIDs) and structured exercise. Dougados et al., Arthritis Rheum. 44:186-185 (2001); Khan, Sem. Arthritis Rheum. 15(Suppl 1):80-84 (1985); Wasner et al., JAMA 246:2168-2172 (1981); Hidding et al., Arthritis Care Res. 6:117-125 (1993); Sweeney et al., J. Rheumatol. 29:763-766 (2002); and Dagfinrud et al., “The Cochrane Database of Systematic Reviews”, Issue 4, Art. No.: CD002822, DOI: 10.1002/14651858.CD002822.pub2 (2004). Attempts to treat ankylosing spondylitis with anti-rheumatic drugs have been disappointing. Sulfasalazine improves SpA-associated peripheral arthritis, but not spinal pain. Clegg et al., Arthritis Rheum. 39:2004-2012 (1996); Clegg et al., Arthritis Rheum. 42:2325-2329 (1999); Dougados et al., Arthritis Rheum. 38:618-627 (1995); and Nissila et al., Arthritis Rheum. 31:1111-1116 (1988). Similarly, methotrexate and leflunomide, while effective in the treatment of rheumatoid arthritis, exhibit minimal efficacy against ankylosing spondylitis. Chen et al., “The Cochrane Database of Systematic Reviews”, Iss. 3, Art. No.: CD004524, DOI: 10.1002/14651858.CD004524.pub2 (2003); Haibel et al., Ann. Rheum Dis. 64:124-126 (2005); and Van Denderen et al., Ann. Rheum. Dis. 63(Suppl 1):397 (2004).

More recently, the use of tumor necrosis factor (TNF) blockers has been attempted and enjoyed limited success. For example, Van der Heijde et al., Arthritis Rheum. 52:582-591 (2005) reported that 61% of a treatment group achieved an ASAS20 response after 24 weeks of treatment with infliximab. See, also, Braun et al., Ann. Rheum. Dis. 64:229-234 (2005); Braun et al., Lancet 359:1187-1193 (2002); and Mease et al., Lancet 356:385-390 (2000). Similarly, recent studies with etanercept have indicated an approximately 60% response rate in the treatment of ankylosing spondylitis where a positive response includes reduced spinal inflammation, back pain, and physical impairment. Brandt et al., Arthritis Rheum. 48:1667-1675 (2003), Davis et al., Arthritis Rheum. 48:3230-3236 (2003); and Gorman et al., N. Engl. J. Med. 346:1349-1356 (2002).

Although preliminary, initial studies with adalimumab, a humanized monoclonal anti-TNF antibody, indicate that this therapy may be comparable to infliximab and etanercept in the treatment of ankylosing spondylitis. Haibel et al., Arthritis Rheum. 50(Suppl):S217 (2004). Additionally, anakinra, a recombinant human interleukin-1 receptor antagonist; bisphosphonates and thalidomide; and antibiotic therapies have been attempted for the treatment of ankylosing spondylitis, but results are inconclusive to date. See, Tan et al., Ann. Rheum. Dis. 63:1041-1045 (2004); Maksymowych et al., Arthritis Rheum. 46:766-773 (2002); and Kvein et al., Ann. Rheum. Dis. 63:1113-1119 (2004).

As a whole, little progress has been made in developing therapeutic regimens for the treatment of ankylosing spondylitis and other spondyloarthritides diseases, in part because the treatments do not prevent bone formation and spinal fusion. In order to gain full control of the disease, therapeutic strategies specifically targeting cartilage and bone formation may be required, either as an alternative to or complementary with existing immunosuppressive therapies. Thus, there remains a need in the art for new modalities for the treatment of bone disorders associated with ankylosing spondylitis and other spondyloarthritides diseases as well as other diseases associated with abnormal bone formation and ossification, including those caused by abnormal expression/activity of bone morphogenic proteins and receptors therefor.

### SUMMARY OF THE INVENTION

The present invention addresses these and other related needs by providing antibodies and other therapeutic proteins directed against bone morphogenic proteins and receptors therefor, nucleic acids encoding such antibodies and therapeutic proteins, methods for preparing anti-BMP and anti-BMPR monoclonal antibodies and other therapeutic proteins, and methods for the treatment of diseases, such as bone diseases and cancers including, but not limited to, fibrodysplasia ossificans progressiva (FOP), progressive osseous heteroplasia (POH), spinal chord injury, blunt trauma resulting in intramuscular hematoma, orthopedic surgery, psoriatic arthritis, osteoarthritis, ankylosing spondylitis, seronegative anthropathies, skeletal hyperpstosis, otosclerosis, stapes ankylosis, bone cancers, prostate cancer and exotoses, artherosclerosis, valvular heart disease, lung cancer, melanoma, hematopoietic cancer, renal cancer, and breast cancer.

Thus, the present invention provides isolated monoclonal antibodies, in particular murine, chimeric, humanized, and fully-human monoclonal antibodies, that bind to one or more bone morphogenic protein and receptors therefor and that exhibit one or more desirable functional property. Such properties include, for example, high affinity specific binding to a human bone morphogenic protein such as BMP2 and/or BMP4 or high affinity specific binding to a human bone morphogenic protein receptor such as BMPR1A, BMPR1B, ACTR1, and/or BMPR2. Also provided are methods for treating a variety bone morphogenic protein-mediated diseases using the antibodies, proteins, and compositions of the present invention.

Antibodies and therapeutic proteins disclosed herein are capable of blocking (a) ligand (i.e., BMP2 and/or BMP4) binding to a cognate receptor (i.e., BMPR1A, BMPR1B, ACTR1, and/or BMPR2) and/or (b) receptor heterodimer formation and/or (c) receptor signaling.

In one aspect, the invention pertains to an isolated monoclonal antibody or an antigen-binding portion thereof, wherein the antibody:
(a) binds to a human bone morphogenic protein (e.g. BMP2, or BMP4) or a receptor therefore (e.g., BMPR1A, BMPR1B, ACTR1, or BMPR2) with a K_{D} of 1×10⁻⁷ M or less; and/or
(b) binds to a cells (e.g., human or CHO), wherein said cell expresses a human bone morphogenic protein and/or a receptor therefor.

In more specific embodiments, the antibody binds to a human bone morphogenic protein or receptor therefor with a K_{D} of 5×10⁻⁸M or less, typically 2×10⁻⁸ M or less, more typically 1×10⁻⁸ M or less, even more typically 6×10⁻⁹ M or less, 3×10⁻⁹ M or less, or 2×10⁻⁹ M or less.

In another embodiment, the invention provides an isolated monoclonal antibody or antigen binding portion thereof, wherein the antibody cross-competes for binding to a bone morphogenic protein, or a receptor therefor, with a reference antibody, wherein the reference antibody:
(a) binds to a human bone morphogenic protein or a receptor therefor with a K_{D} of 1×10⁻⁷ M or less; and/or
(b) binds to a cell that expresses a human bone morphogenic protein and/or a receptor therefor.
   In another embodiment, the invention provides an isolated monoclonal antibody, or antigen binding portion thereof, wherein the antibody cross-competes for binding to BMP2 or BMP4 with a reference antibody comprising:
(a) a heavy chain variable region comprising an amino acid sequence selected from the group consisting of SEQ ID NOs:31, 32, or 33; and
(b) a light chain variable region comprising an amino acid sequence selected from the group consisting of SEQ ID NOs:34, 35, or 36.

In various embodiments, the reference antibody comprises:
(a) a heavy chain variable region comprising the amino acid sequence of SEQ ID NO:31; and
(b) a light chain variable region comprising the amino acid sequence of SEQ ID NO:34;

or the reference antibody comprises:
(a) a heavy chain variable region comprising the amino acid sequence of SEQ ID NO:32; and
(b) a light chain variable region comprising the amino acid sequence of SEQ ID NO:35.

or the reference antibody comprises:
(a) a heavy chain variable region comprising the amino acid sequence of SEQ ID NO:33; and
(b) a light chain variable region comprising the amino acid sequence of SEQ ID NO:36.

In another aspect, the invention pertains to an isolated monoclonal antibody, or an antigen-binding portion thereof, comprising a heavy chain variable region that is the product of or derived from a human V_{H} 3-33 gene, wherein the antibody specifically binds BMP2 or BMP4. The invention also provides an isolated monoclonal antibody, or an antigen-binding portion thereof, comprising a heavy chain variable region that is the product of or derived from a human V_{H} 4-34 gene, wherein the antibody specifically binds BMP2 or BMP4. The invention also provides an isolated monoclonal antibody, or an antigen-binding portion thereof, comprising a heavy chain variable region that is the product of or derived from a human V_{H} 4-59 gene, wherein the antibody specifically binds BMP2 or BMP4. The invention still further provides an isolated monoclonal antibody, or an antigen-binding portion thereof, comprising a light chain variable region that is the product of or derived from a human V_{K} A27 gene, wherein the antibody specifically binds BMP2 or BMP4. The invention even further provides an isolated monoclonal antibody, or an antigen-binding portion thereof, comprising a light chain variable region that is the product of or derived from a human V_{K} L6 gene, wherein the antibody specifically binds BMP2 or BMP4. The invention even further provides an isolated monoclonal antibody, or an antigen-binding portion thereof, comprising a light chain variable region that is the product of or derived from a human V_{K} L15 gene, wherein the antibody specifically binds BMP2 or BMP4.

In a preferred embodiment, the invention provides an isolated monoclonal antibody, or an antigen-binding portion thereof, comprising:
(a) a heavy chain variable region of a human V_{H} 3-33, 4-34, or 4-59 gene; and
(b) a light chain variable region of a human V_{K} A27, L6, or V_{K} L15;

wherein the antibody specifically binds to BMP2 or BMP4.

In a preferred embodiment, the antibody comprises a heavy chain variable region of a human V_{H} 4-59 gene and a light chain variable region of a human V_{K} A27 gene. In another preferred embodiment, the antibody comprises a heavy chain variable region of a human V_{H} 4-34 gene and a light chain variable region of a human V_{K} L6 gene. In another preferred embodiment, the antibody comprises a heavy chain variable region of a human V_{H} 3-33 gene and a light chain variable region of a human V_{K} L15 gene.

In another aspect, the invention provides an isolated monoclonal antibody, or antigen binding portion thereof, comprising:
a heavy chain variable region that comprises CDR1, CDR2, and CDR3 sequences; and a light chain variable region that comprises CDR1, CDR2, and CDR3 sequences, wherein:
(a) the heavy chain variable region CDR3 sequence comprises an amino acid sequence selected from the group consisting of amino acid sequences of SEQ ID NOs:19, 20, and 21, and conservative modifications thereof;
(b) the light chain variable region CDR3 sequence comprises an amino acid sequence selected from the group consisting of amino acid sequence of SEQ ID NOs:28, 29, and 30, and conservative modifications thereof; and
(c) the antibody binds to human BMP2 or BMP4 with a K_{D} of 1×10⁻⁷ M or less.

Preferably, the heavy chain variable region CDR2 sequence comprises an amino acid sequence selected from the group consisting of amino acid sequences of SEQ ID NOs:16, 17, and 18, and conservative modifications thereof; and the light chain variable region CDR2 sequence comprises an amino acid sequence selected from the group consisting of amino acid sequences of SEQ ID NOs:25, 26, and 27, and conservative modifications thereof. Preferably, the heavy chain variable region CDR1 sequence comprises an amino acid sequence selected from the group consisting of amino acid sequences of SEQ ID NOs:13, 14, and 15, and conservative modifications thereof; and the light chain variable region CDR1 sequence comprises an amino acid sequence selected from the group consisting of amino acid sequences of SEQ ID NOs:22, 23, and 24, and conservative modifications thereof.

A preferred combination comprises:
(a) a heavy chain variable region CDR1 comprising SEQ ID NO:13;
(b) a heavy chain variable region CDR2 comprising SEQ ID NO:16;
(c) a heavy chain variable region CDR3 comprising SEQ ID NO:19;
(d) a light chain variable region CDR1 comprising SEQ ID NO:22;
(e) a light chain variable region CDR2 comprising SEQ ID NO:25; and
(f) a light chain variable region CDR3 comprising SEQ ID NO:28.

Another preferred combination comprises:
(a) a heavy chain variable region CDR1 comprising SEQ ID NO:14;
(b) a heavy chain variable region CDR2 comprising SEQ ID NO:17;
(c) a heavy chain variable region CDR3 comprising SEQ ID NO:20;
(d) a light chain variable region CDR1 comprising SEQ ID NO:23;
(e) a light chain variable region CDR2 comprising SEQ ID NO:26; and
(f) a light chain variable region CDR3 comprising SEQ ID NO:29.

Another preferred combination comprises:
(a) a heavy chain variable region CDR1 comprising SEQ ID NO:15;
(b) a heavy chain variable region CDR2 comprising SEQ ID NO:18;
(c) a heavy chain variable region CDR3 comprising SEQ ID NO:21;
(d) a light chain variable region CDR1 comprising SEQ ID NO:24;
(e) a light chain variable region CDR2 comprising SEQ ID NO:27; and
(f) a light chain variable region CDR3 comprising SEQ ID NO:30.

Other preferred antibodies of the invention, or antigen binding portions thereof comprise:
(a) a heavy chain variable region comprising an amino acid sequence selected from the group consisting of SEQ ID NOs:31, 32, and 33; and
(b) a light chain variable region comprising an amino acid sequence selected from the group consisting of SEQ ID NOs:34, 35, and 36;

wherein the antibody specifically binds BMP2 or BMP4.

A preferred combination comprises:
(a) a heavy chain variable region comprising the amino acid sequence of SEQ ID NO:31; and
(b) a light chain variable region comprising the amino acid sequence of SEQ ID NO:34.

Another preferred combination comprises:
(a) a heavy chain variable region comprising the amino acid sequence of SEQ ID NO:32; and
(b) a light chain variable region comprising the amino acid sequence of SEQ ID NO:35.

Another preferred combination comprises:
(a) a heavy chain variable region comprising the amino acid sequence of SEQ ID NO:33; and
(b) a light chain variable region comprising the amino acid sequence of SEQ ID NO:36.

In another aspect of the invention, antibodies, or antigen-binding portions thereof, are provided that compete for binding to BMP2 or BMP4 with any of the aforementioned antibodies.

The antibodies of the invention can be, for example, full-length antibodies, typically of an IgG1, IgG2, IgG3, or IgG4 isotype. Alternatively, the antibodies can be antibody fragments, such as Fab, Fab′, or Fab′₂ fragments or single chain antibodies (e.g., scFv).

The invention also provides an immunoconjugate comprising an antibody of the invention or antigen-binding portion thereof, linked to a therapeutic agent, such as a cytotoxin or a radioactive isotope. The invention also provides a bispecific molecule comprising an antibody or antigen-binding portion thereof, of the invention, linked to a second functional moiety having a different binding specificity than said antibody or antigen binding portion thereof. The invention also provides Affibodies, domain antibodies, Nanobodies, UniBodies, DARPins, Anticalins, Avimers, Versabodies, and Duocalins directed to BMP2, BMP4, BMPR1A, BMPR1B, ACTR1, or BMPR2.

Compositions comprising an antibody or antigen-binding portion thereof or immunoconjugate or bispecific molecule of the invention and a pharmaceutically acceptable carrier are also provided.

Nucleic acid molecules encoding the antibodies or antigen-binding portions thereof are also encompassed by the present invention, as are expression vectors comprising such nucleic acids, host cells comprising such expression vectors, and methods for making anti-BMP2, anti-BMP4, anti-BMPR1A, anti-BMPR1B, anti-ACTR1, and/or anti-BMPR2 antibodies using such host cells.

Moreover, the present invention provides a transgenic mouse comprising human immunoglobulin heavy and light chain transgenes, wherein the mouse expresses an antibody of the invention, as well as hybridomas prepared from such a mouse, wherein the hybridoma produces the antibody of the invention.

In yet another aspect, the invention provides a method for treating or preventing a disease characterized by growth of bone and/or tumor cells expressing BMP2, BMP4, BMPR1A, BMPR1B, ACTR1, and/or BMPR2, comprising administering to a subject an anti-BMP2, anti-BMP4, anti-BMPR1A, anti-BMPR1B, anti-ACTR1, and/or anti-BMPR2 human antibody of the present invention in an amount effective to treat or prevent the disease. The disease can be a bone disease and/or can be a cancer.

In yet another aspect, the invention provides a method of treating an autoimmune disorder, comprising administering to a subject an anti-BMP2, anti-BMP4, anti-BMPR1A, anti-BMPR1B, anti-ACTR1, and/or anti-BMPR2 human antibody of the present invention in an amount effective to treat the disorder.

Other features and advantages of the instant invention will be apparent from the following detailed description and examples which should not be construed as limiting. The contents of all references, GenBank accession numbers, patents and published patent applications cited throughout this application are expressly incorporated herein by reference.

### BRIEF DESCRIPTION OF THE DRAWINGS

FIG. 1a shows the nucleotide sequence (SEQ ID NO:37) and amino acid sequence (SEQ ID NO:31) of the heavy chain variable region of the 6H4 human monoclonal antibody. The CDR1 (SEQ ID NO:13), CDR2 (SEQ ID NO:16) and CDR3 (SEQ ID NO:19) regions are delineated and the V, D and J germline derivations are indicated.

FIG. 1b shows the nucleotide sequence (SEQ ID NO:40) and amino acid sequence (SEQ ID NO:34) of the light chain variable region of the 6H4 human monoclonal antibody. The CDR1 (SEQ ID NO:22), CDR2 (SEQ ID NO:25) and CDR3 (SEQ ID NO:28) regions are delineated and the V and J germline derivations are indicated.

FIG. 2a shows the nucleotide sequence (SEQ ID NO:38) and amino acid sequence (SEQ ID NO:32) of the heavy chain variable region of the 11F2 human monoclonal antibody. The CDR1 (SEQ ID NO:14), CDR2 (SEQ ID NO:17) and CDR3 (SEQ ID NO:20) regions are delineated and the V and J germline derivations are indicated.

FIG. 2b shows the nucleotide sequence (SEQ ID NO:41) and amino acid sequence (SEQ ID NO:35) of the light chain variable region of the 11F2 human monoclonal antibody. The CDR1 (SEQ ID NO:23), CDR2 (SEQ ID NO:26) and CDR3 (SEQ ID NO:29) regions are delineated and the V and J germline derivations are indicated.

FIG. 3a shows the nucleotide sequence (SEQ ID NO:39) and amino acid sequence (SEQ ID NO:33) of the heavy chain variable region of the 12E3 human monoclonal antibody. The CDR1 (SEQ ID NO:15), CDR2 (SEQ ID NO:18) and CDR3 (SEQ ID NO:21) regions are delineated and the V and J germline derivations are indicated.

FIG. 3b shows the nucleotide sequence (SEQ ID NO:42) and amino acid sequence (SEQ ID NO:36) of the light chain variable region of the 12E3 human monoclonal antibody. The CDR1 (SEQ ID NO:24), CDR2 (SEQ ID NO:27) and CDR3 (SEQ' ID NO:30) regions are delineated and the V and J germline derivations are indicated.

FIG. 4 shows the alignment of the amino acid sequence of the heavy chain variable region of 6114 (SEQ ID NO:31) with the human germline V_{H} 4-34 amino acid sequence (SEQ ID NO:51), the human germline D_{H} 3-10 amino acid sequence (SEQ ID NO:52), located between the V and J regions, and the human germline J_{H} JH1 amino acid sequence (SEQ ID NO:53).

FIG. 5 shows the alignment of the amino acid sequence of the heavy chain variable region of 11F2 (SEQ ID NO:32) with the human germline V_{H} 4-59 amino acid sequence (SEQ ID NO:43), the human germline D_{H} 2-2 amino acid sequence (SEQ ID NO:45), located between the V and J regions, and the human germline J_{H} JH5b amino acid sequence (SEQ ID NO:46).

FIG. 6 shows the alignment of the amino acid sequence of the heavy chain variable region of 12E3 (SEQ ID NO:33) with the human germline V_{H} 3-33 amino acid sequences (SEQ ID NO:44) and the human germline J_{H} JH6b amino acid sequence (SEQ ID NO:47).

FIG. 7 shows the alignment of the amino acid sequence of the light chain variable region of 6H4 (SEQ ID NO:34) with the human germline V_{K} L6 amino acid sequence (SEQ ID NO:54) and the human germline J_{K} JK2 amino acid sequence (SEQ ID NO:55).

FIG. 8 shows the alignment of the amino acid sequence of the light chain variable region of 11F2 (SEQ ID NO:35) with the human germline V_{K} A27 amino acid sequence (SEQ ID NO:48) and the human germline J_{K} JK4 amino acid sequence (SEQ ID NO:50).

FIG. 9 shows the alignment of the amino acid sequence of the light chain variable region of 12E3 (SEQ ID NO:36) with the human germline V_{K} L15 amino acid sequence (SEQ ID NO:49) and the human germline J_{K} JK4 amino acid sequence (SEQ ID NO:50).

FIG. 10 shows anti-BMP2/4 monoclonal antibodies blocking BMP4 binding to type-II (FIG. 10a) and type-1 (FIG. 10b) BMP receptors by Biacore analysis.

FIG. 11 shows inhibition of BMP2 and BMP4 signaling by anti-BMP2/4 antibodies. C2C12 cells were incubated with recombinant human BMP2 (FIG. 11a) or BMP4 (FIG. 11b) and varying concentrations of five different neutralizing anti-BMP2/4 monoclonal antibodies or IgG1 control mAb. Cells were fixed, lysed, and assayed for alkaline phosphatase activity.

FIG. 12 shows by densitometry scanning that bone formation is significantly reduced by the anti-BMP2 monoclonal antibodies of the invention.

### BRIEF DESCRIPTION OF THE BONE MORPHOGENIC PROTEIN SEQUENCES

SEQ ID NO: 1 is the nucleotide sequence of a cDNA encoding human bone morphogenic protein 2 (BMP2) disclosed under GenBank Accession No. NM_{—}001200.

SEQ ID NO: 2 is the amino acid sequence of human bone morphogenic protein 2 (BMP2) encoded by the nucleotide sequence presented in SEQ ID NO: 1.

SEQ ID NO: 3 is the nucleotide sequence of a cDNA encoding human bone morphogenic protein 4 (BMP4) disclosed under GenBank Accession No. NM_{—}130851.

SEQ ID NO: 4 is the amino acid sequence of human bone morphogenic protein 4 (BMP4) encoded by the nucleotide sequence presented in SEQ ID NO: 3.

SEQ ID NO: 5 is the nucleotide sequence of a cDNA encoding human bone morphogenic protein receptor 1A (BMPR1A) disclosed under GenBank Accession No. NM_{—}004329.

SEQ ID NO: 6 is the amino acid sequence of human bone morphogenic protein receptor 1A (BMPR1A) encoded by the nucleotide sequence presented in SEQ ID NO: 5.

SEQ ID NO: 7 is the nucleotide sequence of a cDNA encoding human bone morphogenic protein receptor 1B (BMPR1B) disclosed under GenBank Accession No. NM_{—}001203.

SEQ ID NO: 8 is shows the amino acid sequence of human bone morphogenic protein receptor 1B (BMPR1B) encoded by the nucleotide sequence presented in SEQ ID NO: 7.

SEQ ID NO: 9 is the nucleotide sequence of a cDNA encoding human activin A receptor, type I (ACTR1) disclosed under GenBank Accession No. BC033867.

SEQ ID NO: 10 is the amino acid sequence of human activin A receptor, type I (ACTR1) encoded by the nucleotide sequence presented in SEQ ID NO: 9.

SEQ ID NO: 11 is the nucleotide sequence of a cDNA encoding human bone morphogenic protein receptor 2 (BMPR2) disclosed under GenBank Accession No. NM_{—}001204.

SEQ ID NO: 12 is the amino acid sequence of human bone morphogenic protein receptor 2 (BMPR2) encoded by the nucleotide sequence presented in SEQ ID NO: 11.

### DETAILED DESCRIPTION

The present invention relates to isolated monoclonal antibodies, particularly murine, chimeric, humanized, and fully-human monoclonal antibodies, that bind specifically to one or more bone morphogenic protein (BMP) or one or more bone morphogenic protein receptor (BMPR) and/or activin A receptor (ACTR1) with high affinity. In certain embodiments, antibodies of the present invention are derived from particular heavy and light chain germline sequences and/or comprise particular structural features such as CDR regions comprising particular amino acid sequences. The invention thus provides isolated antibodies, immunoconjugates, bispecific molecules, Affibodies, domain antibodies, Nanobodies, UniBodies, DARPins, Anticalins, Avimers, Versabodies, and Duocalins, methods of making said molecules, and pharmaceutical compositions comprising said molecules and a pharmaceutical carrier. The invention also relates to methods for using said antibodies, immunoconjugates, bispecific molecules, Affibodies, domain antibodies, Nanobodies, UniBodies, DARPins, Anticalins, Avimers, Versabodies, and Duocalins to treat diseases with abnormal bone formation and cancers.

### Definitions

In order that the present invention may be more readily understood, certain terms are first defined. Additional definitions are set forth throughout the detailed description.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or “antibody portion”), as used herein, refers to one or more fragments of an antibody that retain the ability to specifically bind to an antigen (exemplified herein by BMP2, BMP4, BMPR1A, BMPR1B, ACTR1, and/or BMPR2). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fab′ fragment, which is essentially an Fab with part of the hinge region (see, Fundamental Immunology (Paul ed., 3^{rd} ed. 1993); (iv) a Fd fragment consisting of the V_{H} and C_{H1} domains; (v) a Fv fragment consisting of the V_{L} and V_{H} domains of a single arm of an antibody, (vi) a dAb fragment (Ward et al., (1989) Nature 341:544-546), which consists of a V_{H} domain; and (vii) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art and the fragments are screened for utility in the same manner as are intact antibodies.

An “isolated antibody”, as used herein, is intended to refer to an antibody that is substantially free of other antibodies having different antigenic specificities (e.g., an isolated antibody that specifically binds BMP2, BMP4, BMPR1A, BMPR1B, ACTR1, and/or BMPR2 is substantially free of antibodies that specifically bind antigens other than any one or more of these six proteins). An isolated antibody that specifically binds BMP2, BMP4, BMPR1A, BMPR1B, ACTR1, and/or BMPR2 may, however, have cross-reactivity to other antigens, such as BMP2, BMP4, BMPR1A, BMPR1B, ACTR1, and/or BMPR2 molecules from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody” or “human sequence antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies may include later modifications, including natural or synthetic modifications. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody”, which may include the term “sequence” after “human”, refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds” is intended to refer to an antibody that binds to its cognate antigen with a K_{D} of 1×10⁻⁷ or less, particularly 5×10⁻⁸ M or less, more particular 1×10⁻⁸ M or less, more particularly still 6×10⁻⁹ M or less, more particularly 3×10⁻⁹ M or less, even more particularly 2×10⁻⁹ M or less.

The term “does not substantially bind” to a protein or cells, as used herein, means does not bind or does not bind with a high affinity to the protein or cells, i.e. binds to the protein or cells with a K_{D} of 1×10⁻⁶ M or more, more preferably 1×10⁻⁵ M or more, more preferably 1×10⁻⁴ M or more, more preferably 1×10⁻³ M or more, even more preferably 1×10⁻² M or more.

The term “K_{assoc}” or “Kₐ”, as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e. K_{d}/Kₐ) and is expressed as a molar concentration (M). K_{D} values for antibodies can be determined using methods well established in the art. A preferred method for determining the K_{D} of an antibody is by using surface plasmon resonance, typically using a biosensor system such as a Biacore® system.

As used herein, the term “high affinity” for an IgG antibody refers to an antibody having a K_{D} of 10⁻⁷ M or less, more typically 10⁻⁸ M or less, more typically 10⁻⁹ M or less, and even more typically 10⁻¹⁰ M or less for a target antigen. However, “high affinity” binding can vary for other antibody isotypes. For example, “high affinity” binding for an IgM isotype refers to an antibody having a K_{D} of 10⁻⁷ M or less, more typically 10⁻⁸ M or less, even more typically 10⁻⁹ M or less.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, fish, reptiles, etc.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

A “signal transduction pathway” refers to the biochemical relationship between a variety of signal transduction molecules that play a role in the transmission of a signal from one portion of a cell to another portion of a cell. As used herein, the phrase “cell surface receptor” includes, for example, molecules and complexes of molecules capable of receiving a signal and the transmission of such a signal across the plasma membrane of a cell. An example of a “cell surface receptor” of the present invention are the BMPR1A, BMPR1B, ACTR1, and BMPR2 receptors.

As used herein, the term “BMP2” is used to refer to human bone morphogenic protein 2. The nucleotide sequence of human BMP2 is publicly available by reference to GenBank Accession No. NM_{—}001200 and is disclosed herein as SEQ ID NO. 1. The corresponding amino acid sequence of BMP2 is presented herein as SEQ ID NO: 2.

As used herein, the term “BMP4” is used to refer to human bone morphogenic protein 4. The nucleotide sequence of human BMP4 is publicly available by reference to GenBank Accession No. NM_{—}130851 and is disclosed herein as SEQ ID NO. 3. The corresponding amino acid sequence of BMP4 is presented herein as SEQ ID NO: 4.

As used herein, the term “BMPR1A” (aka Alk3) is used to refer to human bone morphogenic protein receptor 1A. The nucleotide sequence of human BMPR1A is publicly available by reference to GenBank Accession No. NM_{—}004329 and is disclosed herein as SEQ ID NO. 5. The corresponding amino acid sequence of BMPR1A is presented herein as SEQ ID NO: 6.

As used herein, the term “BMPR1B” (aka Alk6) is used to refer to human bone morphogenic protein receptor 1B. The nucleotide sequence of human BMPR1B is publicly available by reference to GenBank Accession No. NM_{—}001203 and is disclosed herein as SEQ ID NO. 7. The corresponding amino acid sequence of BMPR1B is presented herein as SEQ ID NO: 8.

As used herein, the term “ACTR1” is used to refer to human activin A receptor 1. The nucleotide sequence of human ACTR1 is publicly available by reference to GenBank Accession No. BC033867 and is disclosed herein as SEQ ID NO. 9. The corresponding amino acid sequence of ACTR1 is presented herein as SEQ ID NO: 10.

As used herein, the term “BMPR2” is used to refer to human bone morphogenic protein receptor 2. The nucleotide sequence of human BMPR2 is publicly available by reference to GenBank Accession No. NM_{—}001204 and is disclosed herein as SEQ ID NO. 11. The corresponding amino acid sequence of BMPR2 is presented herein as SEQ ID NO: 12.

Various aspects of the invention are described in further detail in the following subsections.

### Antibodies Directed Against BMP2, BMP4, BMPR1A, BMPR1B, ACTR1, and BMPR2

Antibodies of the present invention ate characterized by particular functional features or properties. For example, within certain embodiments, antibodies bind specifically to one or more bone morphogenic protein selected from human BMP2 and human BMP4. Within alternative embodiments, antibodies bind specifically to one or more bone morphogenic protein receptor selected from BMPR1A, BMPR1B, and BMPR2 and/or one or more activin type 1 receptor selected from ACTR1. Typically, an antibody of the invention binds with high affinity, for example with a K_{D} of 5×10⁻⁷ M or less, even more typically 5.5×10⁻⁹ or less, even more typically 3×10⁻⁹ or less, even more typically 2×10⁻⁹ or less or even more typically 1.5×10⁻⁹ or less.

In one embodiment, the antibodies preferably bind to an antigenic epitope present in BMP2 or BMP4, which epitope is not present in other proteins. The antibodies typically bind to BMP2 or BMP4 but does not bind to other proteins, or binds to other proteins with a low affinity, such as with a K_{D} of 1×10⁻⁶ M or more, more preferably 1×10⁻⁵ M or more, more preferably 1×10⁻⁴ M or more, more preferably 1×10⁻³M or more, even more preferably 1×10⁻² M or more. Preferably, the antibodies do not substantially bind to related proteins, for example, the antibodies do not substantially bind to BMP3 or BMP8b.

Standard assays to evaluate the binding ability of the antibodies toward one or more bone morphogenic proteins or receptors therefor are known in the art including, for example, ELISAs, Western blots, flow cytometry and RIAs. Suitable assays are described in detail in the Examples. The binding kinetics (e.g., binding affinity) of the antibodies also can be assessed by standard assays known in the art, such as by ELISA, Scatchard and Biacore analysis. As another example, the antibodies of the present invention may bind to a bone cell such as a prechondrocyte and/or a chondrocyte.

### Human Monoclonal Antibodies Directed against BMP2, BMP4, BMPR1A, BMPR1B, ACTR1, and/or BMPR2

It will be understood that antibodies directed to BMP2 may desirably cross-react with BMP4 and antibodies directed to BMP4 may desirably cross-react with BMP2. Similarly, antibodies directed against any one of BMPR1A, BMPR1B, ACTR1, and BMPR2 may desirably cross-react with any of the alternative BMP and/or ACV receptors. Thus, the present invention contemplates that V_{H} and V_{L} sequences may be advantageously “mixed and matched” to create other antigen-specific binding molecules within the scope of the presently claimed invention. Specific binding of such “mixed and matched” antibodies can be tested using the binding assays described above and in the Examples (e.g., FACS or ELISAs). Typically, when V_{H} and V_{L} chains are mixed and matched, a V_{H} sequence from a particular V_{H}/V_{L} pairing is replaced with a structurally similar V_{H} sequence. Likewise, typically a V_{L} sequence from a particular V_{H}/V_{L} pairing is replaced with a structurally similar V_{L} sequence.

Preferred antibodies of the invention were isolated and structurally characterized as described in Examples 1 and 2 and include the human monoclonal antibodies 6H4, 11F2, and 12E3. The V_{H} amino acid sequences of 6H4, 11F2, and 12E3 are shown in SEQ ID NOs:31, 32, and 33, respectively. The V_{L} amino acid sequences of 6H4, 11F2, and 12E3 are shown in SEQ ID NOs:34, 35, and 36, respectively.

In one aspect, the invention provides an isolated monoclonal antibody, or antigen binding portion thereof comprising:
(a) a heavy chain variable region comprising an amino acid sequence selected from the group consisting of SEQ ID NOs:31, 32, and 33; and
(b) a light chain variable region comprising an amino acid sequence selected from the group consisting of SEQ ID NOs:34, 35, and 36;

wherein the antibody specifically binds BMP2 or BMP4, preferably human BMP2 or BMP4.

Preferred heavy and light chain combinations include:
(a) a heavy chain variable region comprising the amino acid sequence of SEQ ID NO:31; and (b) a light chain variable region comprising the amino acid sequence of SEQ ID NO:34; or
(b) a heavy chain variable region comprising the amino acid sequence of SEQ ID NO:32; and (b) a light chain variable region comprising the amino acid sequence of SEQ ID NO:35; or
(b) a heavy chain variable region comprising the amino acid sequence of SEQ ID NO:33; and (b) a light chain variable region comprising the amino acid sequence of SEQ ID NO:36.

In another aspect, the invention provides antibodies that comprise the heavy chain and light chain CDR1s, CDR2s and CDR3s of 6H4, 11F2, and 12E3, or combinations thereof. The amino acid sequences of the V_{H} CDR1s of 6H4, 11F2, and 12E3 are shown in SEQ ID NOs:13, 14, and 15. The amino acid sequences of the V_{H} CDR2s of 6H4, 11F2, and 12E3 are shown in SEQ ID NOs:16, 17, and 18. The amino acid sequences of the V_{H} CDR3s of 6H4, 11F2, and 12E3 are shown in SEQ ID NOs:19, 20, and 21. The amino acid sequences of the V_{K} CDR1s of 6H4, 11F2, and 12E3 are shown in SEQ ID NOs:22, 23, and 24. The amino acid sequences of the V_{K} CDR2s of 6H4, 11F2, and 12E3 are shown in SEQ ID NOs:25, 26, and 27. The amino acid sequences of the V_{K} CDR3s of 6H4, 11F2, and 12E3 are shown in SEQ ID NOs:28, 29, and 30. The CDR regions are delineated using the Kabat system (Kabat, E. A., et al. (1991) Sequences of Proteins of Immunological Interest, Fifth Edition, U.S. Department of Health and Human Services, NIH Publication No. 91-3242).

Given that each of the monoclonal antibodies provided herein can bind (1) to a bone morphogenic protein selected from BMP2 and BMP4 or (2) to a bone morphogenic protein receptor selected from BMPR1A, BMPR1B, BMPR2 and/or to an activin type 1 receptor selected from ACTR1 and that antigen-binding specificity is provided primarily by the CDR1, CDR2 and CDR3 regions, the V_{H} CDR1, CDR2 and CDR3 sequences and Vₖ CDR1, CDR2 and CDR3 sequences can be “mixed and matched” (i.e. CDRs from different antibodies can be mixed and matched, although each antibody must contain a V_{H} CDR1, CDR2 and CDR3, and a Vₖ CDR1, CDR2 and CDR3) to create other antigen-specific binding molecules of the invention. Binding of such “mixed and matched” antibodies can be tested using the binding assays described above and in the Examples (e.g., FACS, ELISAs, Biacore analysis). Typically, when V_{H} CDR sequences are mixed and matched, the CDR1, CDR2, and/or CDR3 sequence from a particular V_{H} sequence is replaced with a structurally similar CDR sequence(s). Likewise, when Vₖ CDR sequences are mixed and matched, the CDR1, CDR2 and/or CDR3 sequence from a particular Vₖ sequence typically is replaced with a structurally similar CDR sequence(s). It will be readily apparent to the ordinarily skilled artisan that novel V_{H} and V_{L} sequences can be created by substituting one or more V_{H} and/or V_{L} CDR region sequences with structurally similar sequences from the CDR sequences disclosed herein for monoclonal antibodies of the present invention.

In another aspect, the invention provides an isolated monoclonal antibody or antigen binding portion thereof comprising:
(a) a heavy chain variable region CDR1;
(b) a heavy chain variable region CDR2;
(c) a heavy chain variable region CDR3;
(d) a light chain variable region CDR1;
(e) a light chain variable region CDR2; and
(f) a light chain variable region CDR3; wherein each heavy chain variable region CDR1, CDR2, and/or CDR3 and each light chain variable region CDR1, CDR 2, and/or CDR3 comprises an amino acid sequence selected from one, two, three, four, five, or six bone morphogenic protein receptor binding antibody(ies); and wherein the antibody(ies) specifically bind(s) to BMP2 and/or BMP4 (typically human BMP2 and/or BMP4).
   Accordingly, in another aspect, the invention provides an isolated monoclonal antibody, or antigen binding portion thereof comprising:
(a) a heavy chain variable region CDR1 comprising an amino acid sequence selected from the group consisting of SEQ ID NOs:13, 14, and 15;
(b) a heavy chain variable region CDR2 comprising an amino acid sequence selected from the group consisting of SEQ ID NOs:16, 17, and 18;
(c) a heavy chain variable region CDR3 comprising an amino acid sequence selected from the group consisting of SEQ ID NOs:19, 20, and 21;
(d) a light chain variable region CDR1 comprising an amino acid sequence selected from the group consisting of SEQ ID NOs:22, 23, and 24;
(e) a light chain variable region CDR2 comprising an amino acid sequence selected from the group consisting of SEQ ID NOs:25, 26, and 27; and
(f) a light chain variable region CDR3 comprising an amino acid sequence selected from the group consisting of SEQ ID NOs:28, 29, and 30;

wherein the antibody specifically binds BMP2 or BMP4, preferably human BMP2 or BMP4.

In a preferred embodiment, the antibody comprises:
(a) a heavy chain variable region CDR1 comprising SEQ ID NO:13;
(b) a heavy chain variable region CDR2 comprising SEQ ID NO:16;
(c) a heavy chain variable region CDR3 comprising SEQ ID NO:19;
(d) a light chain variable region CDR1 comprising SEQ ID NO:22;
(e) a light chain variable region CDR2 comprising SEQ ID NO:25; and
(f) a light chain variable region CDR3 comprising SEQ ID NO:28.

In another preferred embodiment, the antibody comprises:
(a) a heavy chain variable region CDR1 comprising SEQ ID NO:14;
(b) a heavy chain variable region CDR2 comprising SEQ ID NO:17;
(c) a heavy chain variable region CDR3 comprising SEQ ID NO:20;
(d) a light chain variable region CDR1 comprising SEQ ID NO:23;
(e) a light chain variable region CDR2 comprising SEQ ID NO:26; and
(f) a light chain variable region CDR3 comprising SEQ ID NO:29.

In another preferred embodiment, the antibody comprises:
(a) a heavy chain variable region CDR1 comprising SEQ ID NO:15;
(b) a heavy chain variable region CDR2 comprising SEQ ID NO:18;
(c) a heavy chain variable region CDR3 comprising SEQ ID NO:21;
(d) a light chain variable region CDR1 comprising SEQ ID NO:24;
(e) a light chain variable region CDR2 comprising SEQ ID NO:27; and
(f) a light chain variable region CDR3 comprising SEQ ID NO:30.

It is well known in the art that the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, for example, Klimka et al., British J. of Cancer 83(21:252-260 (2000) [describing the production of a humanized anti-CD30 antibody using only the heavy chain variable domain CDR3 of murine anti-CD30 antibody Ki-4]; Beiboer et al., J. Mol. Biol. 296:833-849 (2000) [describing recombinant epithelial glycoprotein-2 (EGP-2) antibodies using only the heavy chain CDR3 sequence of the parental murine MOC-31 anti-EGP-2 antibody]; Rader et al., Proc. Natl. Acad. Sci. U.S.A. 95:8910-8915 (1998) [describing a panel of humanized anti-integrin αᵥβ₃ antibodies using a heavy and light chain variable CDR3 domain of a murine anti-integrin αᵥβ₃ antibody LM609 wherein each member antibody comprises a distinct sequence outside the CDR3 domain and capable of binding the same epitope as the parent murine antibody with affinities as high or higher than the parent murine antibody]; Barbas et al., J. Am. Chem. Soc. 116:2161-2162 (1994) [disclosing that the CDR3 domain provides the most significant contribution to antigen binding]; Barbas et al., Proc. Natl. Acad. Sci. U.S.A. 92:2529-2533 (1995) [describing the grafting of heavy chain CDR3 sequences of three Fabs (SI-1, SI-40, and SI-32) against human placental DNA onto the heavy chain of an anti-tetanus toxoid Fab thereby replacing the existing heavy chain CD3 and demonstrating that the CDR3 domain alone conferred binding specificity]; and Ditzel et al., J. Immunol. 157:739-749 (1996) [describing grafting studies wherein transfer of only the heavy chain CDR3 of a parent polyspecific Fab LNA3 to a heavy chain of a monospecific IgG tetanus toxoid-binding Fab p313 antibody was sufficient to retain binding specificity of the parent Fab]. Each of these references is hereby incorporated by reference in its entirety.

Accordingly, within certain aspects, the present invention provides monoclonal antibodies comprising one or more heavy and/or light chain CDR3 domain from a non-human antibody, such as a mouse or rat antibody, wherein the monoclonal antibody is capable of specifically binding to BMP2 and/or BMP4 (typically human BMP2 and/or BMP4) or to BMPR1A, BMPR1B, ACTR1, and/or BMPR2 (typically human BMPR1A, BMPR1B, ACTR1, and/or BMPR2). Within some embodiments, such inventive antibodies comprising one or more heavy and/or light chain CDR3 domain from a non-human antibody (a) are capable of competing for binding with; (b) retain the functional characteristics; (c) bind to the same epitope; and/or (d) have a similar binding affinity of the corresponding parental non-human antibody.

Within other aspects, the present invention provides monoclonal antibodies comprising one or more heavy and/or light chain CDR3 domain from a first human antibody, such as, for example, a human antibody obtained from a non-human animal, wherein the first human antibody is capable of specifically binding to BMP2 and/or BMP4 (typically human BMP2 and/or BMP4) or to BMPR1A, BMPR1B, ACTR1, and/or BMPR2 (typically human BMPR1A, BMPR1B, ACTR1, and/or BMPR2) and wherein the CDR3 domain from the first human antibody replaces a CDR3 domain in a human antibody that is lacking binding specificity for BMP2 and/or BMP4 or to BMPR1A, BMPR1B, ACTR1, and/or BMPR2 to generate a second human antibody that is capable of specifically binding to BMP2 and/or BMP4 or to BMPR1A, BMPR1B, ACTR1, and/or BMPR2, respectively. Within some embodiments, such inventive antibodies comprising one or more heavy and/or light chain CDR3 domain from the first human antibody (a) are capable of competing for binding with; (b) retain the functional characteristics; (c) bind to the same epitope; and/or (d) have a similar binding affinity as the corresponding parental first human antibody.

### Antibodies Having Particular Germline Sequences

In certain embodiments, an antibody of the present invention comprises a heavy chain variable region from a particular germline heavy chain immunoglobulin gene and/or a light chain variable region from a particular germline light chain immunoglobulin gene.

As used herein, a human antibody comprises heavy or light chain variable regions that is “the product of” or “derived from” a particular germline sequence if the variable regions of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e. greatest % identity) to the sequence of the human antibody.

For example, in a preferred embodiment, the invention provides an isolated monoclonal antibody, or an antigen-binding portion thereof, comprising a heavy chain variable region that is the product of or derived from a human V_{H} 4-59 gene, wherein the antibody specifically binds BMP2 or BMP4. In another preferred embodiment, the invention provides an isolated monoclonal antibody, or an antigen-binding portion thereof, comprising a heavy chain variable region that is the product of or derived from a human V_{H} 4-34 gene, wherein the antibody specifically binds BMP2 or BMP4. In another preferred embodiment, the invention provides an isolated monoclonal antibody, or an antigen-binding portion thereof, comprising a heavy chain variable region that is the product of or derived from a human V_{H} 3-33 gene, wherein the antibody specifically binds BMP2 or BMP4. In another preferred embodiment, the invention provides an isolated monoclonal antibody, or an antigen-binding portion thereof, comprising a heavy chain variable region that is the product of or derived from a human V_{H} 1-69 gene, wherein the antibody specifically binds BMP2 or BMP4.

In another example, in a preferred embodiment, the invention provides an isolated monoclonal antibody, or an antigen-binding portion thereof, comprising a light chain variable region that is the product of or derived from a human V_{K} A27 gene, wherein the antibody specifically binds BMP2 or BMP4. In yet another preferred embodiment, the invention provides an isolated monoclonal antibody, or an antigen-binding portion thereof, comprising a light chain variable region that is the product of or derived from a human V_{K} L15 gene, wherein the antibody specifically binds BMP2 or BMP4. In yet another preferred embodiment, the invention provides an isolated monoclonal antibody, or an antigen-binding portion thereof, comprising a light chain variable region that is the product of or derived from a human V_{K} L6 gene, wherein the antibody specifically binds BMP2 or BMP4.

In another preferred embodiment, the invention provides an isolated monoclonal antibody, or antigen-binding portion thereof, wherein the antibody:
(a) comprises a heavy chain variable region that is the product of or derived from a human V_{H} 4-59, 4-34, or 3-33 gene (which genes encode the amino acid sequences set forth in SEQ ID NOs:43, 51 and 44, respectively);
(b) comprises a light chain variable region that is the product of or derived from a human V_{K} A27, L6, or L15 gene (which genes encode the amino acid sequences set forth in SEQ ID NOs:48, 54, and 49, respectively); and
(c) specifically binds to BMP2 or BMP4, preferably human BMP2 or BMP4.

An example of an antibody having V_{H} and V_{K} of V_{H} 4-34 and V_{K} L6, respectively, is 6H4. An example of an antibody having a V_{H} and V_{K} of V_{H} 4-59 and V_{K} A27, respectively, is 11F2. An example of an antibody having V_{H} and V_{K} of V_{H} 3-33 and V_{K} L15, respectively, is 12E3.

A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95% or even at least 96%, 97%, 98% or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5 or even no more than 4, 3, 2 or 1 amino acid difference from the amino acid sequence encoded by the germline immunoglobulin gene.

### Homologous Antibodies

In yet another embodiment, an antibody of the invention comprises heavy and light chain variable regions comprising amino acid sequences that are homologous to the amino acid sequences of the antibodies described herein and wherein the antibodies retain the desired functional properties of the antibodies of the present invention.

For example, the present invention provides an isolated monoclonal antibody or antigen binding portion thereof, comprising a heavy chain variable region and a light chain variable region, wherein:
(a) the heavy chain variable region comprises an amino acid sequence that is at least 80% homologous to an amino acid sequence selected from the group consisting of SEQ ID NOs:31, 32, and 33;
(b) the light chain variable region comprises an amino acid sequence that is at least 80% homologous to an amino acid sequence selected from the group consisting of SEQ ID NOs:34, 35, and 36; and
(c) the antibody binds to human BMP2 or BMP4 with a K_{D} of 1'10⁻⁷ M or less.

The antibody may also bind to CHO cells having a cell surface-bound human BMP2 or BMP4. The BMP2 or BMP4 may be bound to receptors or a bivalent entity on the cell surface or may be expressed as fusion proteins with transmembrane domains.

In various embodiments, the antibody can be, for example, a human antibody, a humanized antibody or a chimeric antibody.

In other embodiments, the V_{H} and/or V_{L} amino acid sequences may be 85%, 90%, 95%, 96%, 97%, 98% or 99% homologous to the sequences set forth above. An antibody having V_{H} and V_{L} regions having high (i.e., 80% or greater) homology to the V_{H} and V_{L} regions of the sequences set forth above, can be obtained by mutagenesis (e.g., site-directed or PCR-mediated mutagenesis) of nucleic acid molecules encoding SEQ ID NOs:31, 32, 33, 34, 35, and 36, followed by testing of the encoded altered antibody for retained function using the assays described herein.

The present invention also provides an isolated monoclonal antibody or antigen binding portion thereof, comprising a heavy chain variable region and a light chain variable region, wherein:
(a) the heavy chain variable region comprises an amino acid sequence that is at least 80% identical to the amino acid sequence of a heavy chain variable region presented herein wherein the heavy chain variable region is from an antibody that specifically binds to a bone morphogenic protein receptor selected from BMPR1A, BMPR1B, and/or BMPR2 and/or to an activin type 1 receptor selected from ACTR1;
(b) the light chain variable region comprises an amino acid sequence that is at least 80% homologous to the amino acid sequence of a light chain variable region presented herein wherein the light chain variable region is from an antibody that specifically binds to a bone morphogenic protein receptor selected from BMPR1A, BMPR1B, and/or BMPR2 and/or to an activin type 1 receptor selected from ACTR1; and
(c) the antibody specifically binds to a bone morphogenic protein receptor selected from BMPR1A, BMPR1B, and/or BMPR2 and/or to an activin type 1 receptor selected from ACTR1.

In other embodiments, the V_{H} and/or V_{L} amino acid sequences may be 85%, 90%, 95%, 96%, 97%, 98% or 99% identical to an anti-BMPR1A, BMPR1B, and/or BMPR2 antibody and/or to an anti-ACTR1 sequence set forth herein. An antibody having V_{H} and V_{L} regions having high (i.e. 80% or greater) identity to the V_{H} and V_{L} regions of the sequences set forth herein, can be obtained by mutagenesis (e.g., site-directed or PCR-mediated mutagenesis) of a nucleic acid molecules encoding a V_{H} or a V_{L} region of an anti-BMPR1A, BMPR1B, and/or BMPR2 antibody and/or to an anti-ACTR1.

As used herein, the percent identity between the two sequences is a function of the number of identical positions shared by the sequences (i.e. % homology=# of identical positions/total # of positions×100), taking into account the number of gaps and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)), which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at http://www.gcg.com), using either a Blossum 62 matrix or a PAM250 matrix and a gap weight of 16, 14, 12, 10, 8, 6 or 4 and a length weight of 1, 2, 3, 4, 5 or 6.

Additionally or alternatively, the protein sequences of the present invention can further be used as a “query sequence” to perform a search against public databases to, for example, identify related sequences. Such searches can be performed using the) (BLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

### Antibodies with Conservative Modifications

In certain embodiments, an antibody of the invention comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the exemplary antibodies described herein or conservative modifications thereof and wherein the antibodies retain the desired functional properties of the monoclonal antibodies of the present invention.

Accordingly, the invention provides an isolated monoclonal antibody or antigen binding portion thereof, comprising a heavy chain variable region comprising CDR1, CDR2 and CDR3 sequences and a light chain variable region comprising CDR1, CDR2 and CDR3 sequences, wherein:
(a) the heavy chain variable region CDR3 sequence comprises an amino acid sequence selected from the group consisting of amino acid sequences of SEQ ID NOs: 19, 20, and 21, and conservative modifications thereof;
(b) the light chain variable region CDR3 sequence comprises an amino acid sequence selected from the group consisting of amino acid sequence of SEQ ID NOs:28, 29, and 30, and conservative modifications thereof; and
(c) the antibody binds to human BMP2 or BMP4 with a K_{D} of 1×10⁻⁷ M or less.

The antibody may also bind to CHO cells having a cell surface bound BMP2 or BMP4.

In a preferred embodiment, the heavy chain variable region CDR2 sequence comprises an amino acid sequence selected from the group consisting of amino acid sequences of SEQ ID NOs:16, 17, and 18, and conservative modifications thereof; and the light chain variable region CDR2 sequence comprises an amino acid sequence selected from the group consisting of amino acid sequences of SEQ ID NOs:25, 26, and 27, and conservative modifications thereof.

In another preferred embodiment, the heavy chain variable region CDR1 sequence comprises an amino acid sequence selected from the group consisting of amino acid sequences of SEQ ID NOs:13, 14, and 15, and conservative modifications thereof; and the light chain variable region CDR1 sequence comprises an amino acid sequence selected from the group consisting of amino acid sequences of SEQ ID NOs:22, 23, and 24, and conservative modifications thereof.

In various embodiments, the antibody can be, for example, human antibodies, humanized antibodies or chimeric antibodies.

The present invention also provides an isolated monoclonal antibody or antigen binding portion thereof, comprising a heavy chain variable region comprising CDR1, CDR2 and CDR3 sequences and a light chain variable region comprising CDR1, CDR2 and CDR3 sequences, wherein:
(a) the heavy chain variable region CDR3 sequence comprises an amino acid sequence selected from an anti-BMPR1A, anti-BMPR1B, anti-ACTR1, and anti-BMPR2 monoclonal antibody disclosed herein and conservative modifications thereof;
(b) the light chain variable region CDR3 sequence comprises an amino acid sequence selected from an anti-BMPR1A, anti-BMPR1B, anti-ACTR1, and anti-BMPR2 monoclonal antibody disclosed herein and conservative modifications thereof; and
(c) the antibody specifically binds to BMPR1A, BMPR1B, ACTR1, and/or BMPR2.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for retained function (i.e. the function set forth in (c)) using the functional assays described herein.

### Antibodies that Bind to the Same Epitope as Antibodies of the Invention

In another embodiment, the invention provides antibodies that bind to the same epitope(s) on human BMP2, BMP4, BMPR1A, BMPR1B, ACTR1, and/or BMPR2 as any of the monoclonal antibodies of the present invention (i.e. antibodies that have the ability to cross-compete for binding to BMP2, BMP4, BMPR1A, BMPR1B, ACTR1, and/or BMPR2 with any of the monoclonal antibodies of the invention). In some embodiments, the reference antibody for cross-competition studies can be a monoclonal antibody disclosed herein. Such cross-competing antibodies can be identified based on their ability to cross-compete with an antibody disclosed herein in standard BMP2, BMP4, BMPR1A, BMPR1B, ACTR1, and/or BMPR2 binding assays.

In preferred embodiments, the reference antibody for cross-competition studies can be the monoclonal antibody 6H4 (having V_{H} and V_{L} sequences as shown in SEQ ID NOs:31 and 34, respectively), the monoclonal antibody 11F2 (having V_{H} and V_{L} sequences as shown in SEQ ID NOs:32 and 35, respectively), the monoclonal antibody 12E3 (having V_{H} and V_{L} sequences as shown in SEQ ID NOs:33 and 36, respectively), or any one of the monoclonal antibodies identified in Examples 1 and 2. Such cross-competing antibodies can be identified based on their ability to cross-compete with these antibodies in standard BMP2 or BMP4 binding assays. For example, BIAcore analysis, ELISA assays or flow cytometry may be used to demonstrate cross-competition with the antibodies of the current invention. The ability of a test antibody to inhibit the binding of, for example, 6H4, 11F2, or 12E3, to human BMP2 or BMP4 demonstrates that the test antibody can compete with 6H4, 11F2, or 12E3 for binding to human BMP2 or BMP4 and thus binds to the same epitope on human BMP2 or BMP4 as 6H4, 11F2, or 12E3. In a preferred embodiment, the antibody that binds to the same epitope on human BMP2 or BMP4 as 6H4, 11F2, or 12E3 is a human monoclonal antibody. Such human monoclonal antibodies can be prepared and isolated as described in the Examples.

### Engineered and Modified Antibodies

An antibody of the invention further can be prepared using an antibody having one or more of the V_{H} and/or V_{L} sequences disclosed herein as starting material to engineer a modified antibody, which modified antibody may have altered properties from the starting antibody. An antibody can be engineered by modifying one or more residues within one or both variable regions (i.e. V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al. (1998) Nature 332:323-327; Jones, P. et al. (1986) Nature 321:522-525; Queen, C. et al. (1989) Proc. Natl. Acad. See. U.S.A. 86:10029-10033; U.S. Pat. No. 5,225,539 to Winter and U.S. Pat. Nos. 5,530,101; 5,585,089; 5,693,762 and 6,180,370 to Queen et al.).

Accordingly, another embodiment of the invention pertains to an isolated monoclonal antibody or antigen binding portion thereof, comprising a heavy chain variable region comprising CDR1, CDR2 and CDR3 sequences comprising an amino acid sequence from a first anti-BMP2, anti-BMP4, anti-BMPR1A, anti-BMPR1B, anti-ACTR1, and/or anti-BMPR2 antibody presented herein and a light chain variable region comprising CDR1, CDR2 and CDR3 sequences comprising an amino acid sequence from a second anti-BMP2, anti-BMP4, anti-BMPR1A, anti-BMPR1B, anti-ACTR1 , and/or anti-BMPR2. In a preferred embodiment, an isolated monoclonal antibody, or antigen binding portion thereof, comprising a heavy chain variable region comprising CDR1, CDR2, and CDR3 sequences comprising an amino acid sequence selected from the group consisting of SEQ ID NOs:13, 14, and 15, SEQ ID NOs:16, 17, and 18, and SEQ ID NOs:19, 20, and 21, respectively, and a light chain variable region comprising CDR1, CDR2, and CDR3 sequences comprising an amino acid sequence selected from the group consisting of SEQ ID NOs:22, 23, and 24, SEQ ID NOs:25, 26, and 27, and SEQ ID NOs:28, 29, and 30, respectively. Thus, such antibodies contain the V_{H} and V_{L} CDR sequences of monoclonal antibodies 6H4, 11F2, and 12E3 yet may contain different framework sequences from these antibodies.

Such framework sequences can be obtained, for example, from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V_{H} Segments Reveals a Strong Bias in their Usage” Eur. J. Immunol. 24:827-836; the entire contents of each of which are expressly incorporated herein by reference. As another example, the germline DNA sequences for human heavy and light chain variable region genes can be found in the Genbank database. For example, the following heavy chain germline sequences found in the HCo7 HuMAb mouse are available in the accompanying Genbank accession numbers: 1-69 (NG_{—}0010109, NT_{—}024637 and BC070333), 3-33 (NG_{—}0010109 and NT_{—}024637) and 3-7 (NG_{—}0010109 and NT_{—}024637). As another example, the following heavy chain germline sequences found in the HCo12 HuMAb mouse are available in the accompanying Genbank accession numbers: 1-69 (NG_{—}0010109, NT_{—}024637 and BC070333), 5-51 (NG_{—}0010109 and NT_{—}024637), 4-34 (NG_{—}0010109 and NT_{—}024637), 3-30.3 (CAJ556644) and 3-23 (AJ406678).

Antibody protein sequences are compared against a compiled protein sequence database using one of the sequence similarity searching methods called the Gapped BLAST (Altschul et al. (1997) Nucleic Acids Research 25:3389-3402), which is well known to those skilled in the art. BLAST is a heuristic algorithm in that a statistically significant alignment between the antibody sequence and the database sequence is likely to contain high-scoring segment pairs (HSP) of aligned words. Segment pairs whose scores cannot be improved by extension or trimming is called a hit. Briefly, the nucleotide sequences of VBASE origin (http://vbase.mrc-cpe.cam.ac.uk/vbasel/list2.php) are translated and the region between and including FR1 through FR3 framework region is retained. The database sequences have an average length of 98 residues. Duplicate sequences which are exact matches over the entire length of the protein are removed. A BLAST search for proteins using the program blastp with default, standard parameters except the low complexity filter which is turned off and the substitution matrix of BLOSUM62, filters for top 5 hits yielding sequence matches. The nucleotide sequences are translated in all six frames and the frame with no stop codons in the matching segment of the database sequence is considered the potential hit. This is in turn confirmed using the BLAST program tblastx. This translates the antibody sequence in all six frames and compares those translations to the VBASE nucleotide sequences dynamically translated in all six frames.

The identities are exact amino acid matches between the antibody sequence and the protein database over the entire length of the sequence. The positives (identities+substitution match) are not identical but amino acid substitutions guided by the BLOSUM62 substitution matrix. If the antibody sequence matches two of the database sequences with same identity, the hit with most positives would be decided to be the matching sequence hit.

Preferred framework sequences for use in the antibodies of the invention are those that are structurally similar to the framework sequences used by selected antibodies of the invention, e.g., similar to the V_{H} 4-59 framework sequences (SEQ ID NO:43) and/or the Vr, 3-33 framework sequences (SEQ ID NO:44) and/or the V_{H} 4-34 framework sequences (SEQ ID NO:51) and/or the V_{H} 1-69 framework sequences and/or the V_{K} A27 framework sequences (SEQ ID NO:48) and/or the V_{K} L15 framework sequence (SEQ ID NO:49) and/or the L6 V_{K} framework sequences (SEQ ID NO:54) used by preferred monoclonal antibodies of the invention. The V_{H} CDR1, CDR2, and CDR3 sequences, and the V_{K} CDR1, CDR2, and CDR3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al.).

Another type of variable region modification is to mutate amino acid residues within the V_{H} and/or V_{K} CDR1, CDR2 and/or CDR3 regions to thereby improve one or more binding properties (e.g., affinity) of the antibody of interest. Site-directed mutagenesis or PCR-mediated mutagenesis can be performed to introduce the mutations) and the effect on antibody binding or other functional property of interest, can be evaluated in in vitro or in vivo assays as described herein and provided in the Examples. Typically conservative modifications (as discussed above) are introduced. The mutations may be amino acid substitutions, additions or deletions, but are typically substitutions. Moreover, typically no more than one, two, three, four or five residues within a CDR region are altered.

Accordingly, in another embodiment, the instant disclosure provides isolated anti-BMP2/BMP4 monoclonal antibodies, or antigen binding portions thereof, comprising a heavy chain variable region comprising: (a) a V_{H} CDR1 region comprising an amino acid sequence selected from the group consisting of SEQ ID NOs:13, 14, and 15, or an amino acid sequence having one, two, three, four or five amino acid substitutions, deletions or additions as compared to SEQ ID NOs:13, 14, and 15; (b) a V_{H} CDR2 region comprising an amino acid sequence selected from the group consisting of SEQ ID NOs:16, 17, and 18, or an amino acid sequence having one, two, three, four or five amino acid substitutions, deletions or additions as compared to SEQ ID NOs:16, 17, and 18; (c) a V_{H} CDR3 region comprising an amino acid sequence selected from the group consisting of SEQ ID NOs:19, 20, and 21, or an amino acid sequence having one, two, three, four or five amino acid substitutions, deletions or additions as compared to SEQ ID NOs:19, 20, and 21; (d) a V_{K} CDR1 region comprising an amino acid sequence selected from the group consisting of SEQ ID NOs:22, 23, and 24, or an amino acid sequence having one, two, three, four or five amino acid substitutions, deletions or additions as compared to SEQ ID NOs:22, 23, and 24; (e) a V_{K} CDR2 region comprising an amino acid sequence selected from the group consisting of SEQ ID NOs:25, 26, and 27, or an amino acid sequence having one, two, three, four or five amino acid substitutions, deletions or additions as compared to SEQ ID NOs:25, 26, and 27; and (f) a V_{K} CDR3 region comprising an amino acid sequence selected from the group consisting of SEQ ID NOs:28, 29, and 30, or an amino acid sequence having one, two, three, four or five amino acid substitutions, deletions or additions as compared to SEQ ID NOs:28, 29, and 30.

In yet another embodiment, the invention provides isolated monoclonal antibodies or antigen binding portions thereof, comprising a heavy chain variable region comprising: (a) a V_{H} CDR1 region; (b) a V_{H} CDR2 region; (c) a V_{H} CDR3 region; (d) a V_{K} CDR1 region; (e) a V_{K} CDR2 region; and (f) a V_{K} CDR3 region; wherein each V_{H} CDR1, CDR2, and/or CDR3 region and each V_{K} CDR1, CDR2, and/or CDR3 region is from one, two, three, four, five, or six distinct anti-BMPR1A antibody(ies); one, two, three, four, five, or six distinct anti-BMPR1B antibody(ies); one, two, three, four, five, or six distinct anti-ACTR1 antibody(ies), and/or one, two, three, four, five, or six distinct anti-BMPR2 antibody(ies); or an amino acid sequence having one, two, three, four or five amino acid substitutions, deletions or additions as compared to the one, two, three, four, five, or six distinct anti-BMPR1A antibody(ies); one, two, three, four, five, or six distinct anti-BMPR1B antibody(ies); one, two, three, four, five, or six distinct anti-ACTR1 antibody(ies); and/or one, two, three, four, five, or six distinct anti-BMPR2 antibody(ies).

Engineered antibodies of the invention include those in which modifications have been made to framework residues within V_{H} and/or V_{K}, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. Such “backmutated” antibodies are also intended to be encompassed by the invention.

For example, for 6H4, using the Kabat numbering system, amino acid residue #3 (within FR1) of V_{H} is an histidine (SEQ ID NO:31) whereas this residue in the corresponding V_{H} 4-34 germline sequence is a glutamine (SEQ ID NO:51). To return the framework region sequences to their germline configuration, the somatic mutations can be “backmutated” to the germline sequence by, for example, site-directed mutagenesis or PCR-mediated mutagenesis (e.g., residue #3 (residue #3 of FR1) of the V_{H} of 6H4 can be “backmutated” from histidine to glutamine).

As another example, for 11F2, amino acid residue #27 (within FR1) of V_{H} is an aspartate (SEQ ID NO:32) whereas this residue in the corresponding V_{H} 4-59 germline sequence is a glycine (SEQ ID NO:43). To return the framework region sequences to their germ line configuration, the somatic mutations can be “backmutated” to the germline sequence by, for example, site-directed mutagenesis or PCR-mediated mutagenesis (e.g., residue #27 (residue #27 of FR1) of the V_{H} of 11F2 can be “backmutated” from aspartate to glycine).

As another example, for 11F2, amino acid residue #30 (within FR1) of V_{H} is an arginine (SEQ ID NO:32) whereas this residue in the corresponding V_{H} 4-59 germline sequence is a serine (SEQ ID NO:43). To return the framework region sequences to their germline configuration, the somatic mutations can be “backmutated” to the germline sequence by, for example, site-directed mutagenesis or PCR-mediated mutagenesis (e.g., residue #30 (residue #30 of FR1) of the V_{H} of 11F2 can be “backmutated” from arginine to serine).

As another example, for 11F2, amino acid residue #54 (within CDR2) of V_{H} is a arginine (SEQ ID NO:32) whereas this residue in the corresponding V_{H} 4-59 germline sequence is a serine (SEQ ID NO:43). To return the framework region sequences to their germline configuration, the somatic mutations can be “backmutated” to the germline sequence by, for example, site-directed mutagenesis or PCR-mediated mutagenesis (e.g., residue #54 (residue #5 of CDR2) of the V_{H} of 11F2 can be “backmutated” from arginine to serine).

As another example, for 11F2, amino acid residue #58 (within CDR2) of V_{H} is a histidine (SEQ ID NO:32) whereas this residue in the corresponding V_{H} 4-59 germline sequence is a asparagine (SEQ ID NO:43). To return the framework region sequences to their germline configuration, the somatic mutations can be “backmutated” to the germline sequence by, for example, site-directed mutagenesis or PCR-mediated mutagenesis (e.g., residue #58 (residue #9 of CDR2) of the V_{H} of 11F2 can be “backmutated” from histidine to asparagine).

As another example, for 12E3, amino acid residue #52A (within CDR2) of V_{H} is a aspartate (SEQ ID NO:33) whereas this residue in the corresponding V_{H} 3-33 germline sequence is a tyrosine (SEQ ID NO:44). To return the framework region sequences to their germline configuration, the somatic mutations can be “backmutated” to the germline sequence by, for example, site-directed mutagenesis or PCR-mediated mutagenesis (e.g., residue #52A (residue #4 of CDR2) of the V_{H} of 12E3 can be “backmutated” from aspartate to tyrosine).

As another example, for 12E3, amino acid residue #55 (within CDR2) of V_{H} is a arginine (SEQ ID NO:33) whereas this residue in the corresponding V_{H} 3-33 germline sequence is a serine (SEQ ID NO:44). To return the framework region sequences to their germline configuration, the somatic mutations can be “backmutated” to the germ line sequence by, for example, site-directed mutagenesis or PCR-mediated mutagenesis (e.g., residue #55 (residue #7 of CDR2) of the V_{H} of 12E3 can be “backmutated” from arginine to serine).

As another example, for 12E3, amino acid residue #56 (within CDR2) of V_{H} is a lysine (SEQ ID NO:33) whereas this residue in the corresponding V_{H} 3-33 germline sequence is a asparagine (SEQ ID NO:44). To return the framework region sequences to their germline configuration, the somatic mutations can be “backmutated” to the germline sequence by, for example, site-directed mutagenesis or PCR-mediated mutagenesis (e.g., residue #56 (residue #8 of CDR2) of the V_{H} of 12E3 can be “backmutated” from lysine to asparagine).

As another example, for 11F2, amino acid residue #82 (within FR3) of V_{H} is a methionine (SEQ ID NO:32) whereas this residue in the corresponding V_{H} 4-59 germline sequence is a leucine (SEQ ID NO:43). To return the framework region sequences to their germline configuration, the somatic mutations can be “backmutated” to the germline sequence by, for example, site-directed mutagenesis or PCR-mediated mutagenesis (e.g., residue #82 (residue #17 of FR3) of the V_{H} of 11F2 can be “backmutated” from methionine to leucine).

Another type of framework modification involves mutating one or more residues within the framework region or even within one or more CDR regions, to remove T cell epitopes to thereby reduce the potential immunogenicity of the antibody. This approach is also referred to as “deimmunization” and is described in further detail in U.S. Patent Publication No. 20030153043 by Carr et al.

Engineered antibodies of the invention also include those in which modifications have been made to amino acid residues to increase or decrease immunogenic responses through amino acid modifications that alter interaction of a T-cell epitope on the antibody (see e.g., U.S. Pat. Nos. 6,835,550; 6,897,049 and 6,936,249).

In addition or alternative to modifications made within the framework or CDR regions, antibodies of the invention may be engineered to include modifications within the Fc region, typically to alter one or more functional properties of the antibody, such as serum half-life, complement fixation, Fc receptor binding and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In another embodiment, the antibody is produced as a UniBody as described in WO/2007/059782 which is incorporated herein by reference in its entirety.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region is modified to increase the ability of the antibody to mediate antibody dependent cellular cytotoxicity (ADCC) and/or to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγR1, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A.

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705 and Ms709 lack the fucosyltransferase gene, FUT8 (alpha (1,6) fucosyltransferase), such that antibodies expressed in the Ms704, Ms705 and Ms709 cell lines lack fucose on their carbohydrates. The Ms704, Ms705 and Ms709 FUT8^{-/-} cell lines were created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol Bioeng 87:614-22). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the alpha 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GleNac structures which results in increased ADCC activity of the antibodies (see also Umana et al. (1999) Nat. Biotech. 17:176-180). Alternatively, the fucose residues of the antibody may be cleaved off using a fucosidase enzyme. For example, the fucosidase alpha-L-fucosidase removes fucosyl residues from antibodies (Tarentino, A. L. et al. (1975) Biochem. 14:5516-23).

Defucosylation may also be achieved by the Potelligent™ methodology described by in U.S. Pat. No. 6,946,292 entitled “Cells Producing Antibody Compositions with Increased Antibody Dependent Cytotoxic Activity” (Kyowa Hakko Kogyo Co., Ltd, Tokyo, Japan). By this methodology, a fucosyltransferase-deficient host cell is employed for the production of an antibody having an enhanced level of antibody-dependent cellular cytotoxicity (ADCC) activity.

An alternative approach for generating defucosylated antibodies according to the present invention employs the methodology described by Zhu et al., “Production of Human Monoclonal Antibody in Eggs of Chimeric Chickens,” Nature Biotech. 23:1159-1169 (2005). By this methodology, fully functional monoclonal antibodies are expressed in the egg whites of chimeric chicken eggs with yields of approximately three milligrams per egg [Origen Therapeutics, Burlingame, Calif.]. Antibodies generated in this manner lack terminal sialic acid and fucose residues and, consequently, have up to 100-fold greater antibody-dependent cellular cytotoxicity than antibodies produced in conventional mammalian cell cultures (e.g., Chinese hamster ovary cells). Typically, inventive antibody variable domains are cloned into a vector system (described in Zhu et al.), which is transfected into a chicken embryonic stem cell, and introduced into a chick embryo, thereby producing a chimeric avian bioreactor.

Another modification of the antibodies herein that is contemplated by the invention is pegylation. An antibody can be pegylated to, for example, increase the biological (e.g., serum) half life of the antibody. To pegylate an antibody, the antibody or fragment thereof, typically is reacted with polyethylene glycol (PEG), such as a reactive ester or aldehyde derivative of PEG, under conditions in which one or more PEG groups become attached to the antibody or antibody fragment. Typically, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al.

### Antibody Physical Properties

The antibodies of the present invention may be further characterized by the various physical properties of the BMP2/BMP4 antibodies. Various assays may be used to detect and/or differentiate different classes of antibodies based on these physical properties.

In some embodiments, antibodies of the present invention may contain one or more glycosylation sites in either the light or heavy chain variable region. The presence of one or more glycosylation sites in the variable region may result in increased immunogenicity of the antibody or an alteration of the pK of the antibody due to altered antigen binding (Marshall et al (1972) Annu Rev Biochem 41:673-702; Gala F A and Morrison S L (2004) J Immunol 172:5489-94; Wallick et al (1988) J Exp Med 168:1099-109; Spiro R G (2002) Glycobiology 12:43R-56R; Parekh et al (1985) Nature 316:452-7; Mimura et al. (2000) Mol Immunol 37:697-706). Glycosylation has been known to occur at motifs containing an N—X—S/T sequence. Variable region glycosylation may be tested using a Glycoblot assay, which cleaves the antibody to produce a Fab, and then tests for glycosylation using an assay that measures periodate oxidation and Schiff base formation. Alternatively, variable region glycosylation may be tested using Dionex light chromatography (Dionex-LC), which cleaves saccharides from a Fab into monosaccharides and analyzes the individual saccharide content. In some instances, it is preferred to have an anti-CD19 antibody that does not contain variable region glycosylation. This can be achieved either by selecting antibodies that do not contain the glycosylation motif in the variable region or by mutating residues within the glycosylation motif using standard techniques well known in the art.

In a preferred embodiment, the antibodies of the present invention do not contain asparagine isomerism sites. A deamidation or isoaspartic acid effect may occur on N-G or D-G sequences, respectively. The deamidation or isoaspartic acid effect results in the creation of isoaspartic acid which decreases the stability of an antibody by creating a kinked structure off a side chain carboxy terminus rather than the main chain. The creation of isoaspartic acid can be measured using an iso-quant assay, which uses a reverse-phase HPLC to test for isoaspartic acid.

Each antibody will have a unique isoelectric point (pI), but generally antibodies will fall in the pH range of between 6 and 9.5. The pI for an IgG1 antibody typically falls within the pH range of 7-9.5 and the pI for an IgG4 antibody typically falls within the pH range of 6-8. Antibodies may have a pI that is outside this range. Although the effects are generally unknown, there is speculation that antibodies with a pI outside the normal range may have some unfolding and instability under in vivo conditions. The isoelectric point may be tested using a capillary isoelectric focusing assay, which creates a pH gradient and may utilize laser focusing for increased accuracy (Janini et al (2002) Electrophoresis 23:1605-11; Ma et al. (2001) Chromatographia 53:S75-89; Hunt et al (1998) J Chromatogr A 800:355-67). In some instances, it is preferred to have an anti-CD19 antibody that contains a pI value that falls in the normal range. This can be achieved either by selecting antibodies with a pI in the normal range, or by mutating charged surface residues using standard techniques well known in the art.

Each antibody will have a melting temperature that is indicative of thermal stability (Krishnamurthy R and Manning M C (2002) Curr Pharm Biotechnol 3:361-71). A higher thermal stability indicates greater overall antibody stability in vivo. The melting point of an antibody may be measure using techniques such as differential scanning calorimetry (Chen et al (2003) Pharm Res 20:1952-60; Ghirlando et al (1999) Immunol Lett 68:47-52). T_{M1} indicates the temperature of the initial unfolding of the antibody. T_{M2} indicates the temperature of complete unfolding of the antibody. Generally, it is preferred that the T_{M1} of an antibody of the present invention is greater than 60° C., preferably greater than 65° C., even more preferably greater than 70° C. Alternatively, the thermal stability of an antibody may be measure using circular dichroism (Murray et al. (2002) J. Chromatogr Sci 40:343-9).

In a preferred embodiment, antibodies are selected that do not rapidly degrade. Fragmentation of an anti-CD19 antibody may be measured using capillary electrophoresis (CE) and MALDI-MS, as is well understood in the art (Alexander A J and Hughes D E (1995) Anal Chem 67:3626-32).

In another preferred embodiment, antibodies are selected that have minimal aggregation effects. Aggregation may lead to triggering of an unwanted immune response and/or altered or unfavorable pharmacokinetic properties. Generally, antibodies are acceptable with aggregation of 25% or less, preferably 20% or less, even more preferably 15% or less, even more preferably 10% or less and even more preferably 5% or less. Aggregation may be measured by several techniques well known in the art, including size-exclusion column (SEC) high performance liquid chromatography (HPLC), and light scattering to identify monomers, dimers, trimers or multimers.

### Methods of Engineering Antibodies

As discussed above, the anti-BMP2, anti-BMP4, anti-BMPR1A, anti-BMPR1B, anti-ACTR1, and/or anti-BMPR2 antibodies having V_{H} and V_{K} sequences disclosed herein can be used to create new anti-BMP2, anti-BMP4, anti-BMPR1A, anti-BMPR1B, anti-ACTR1, and/or anti-BMPR2 antibodies by modifying the VH and/or V_{K} sequences or the constant region(s) attached thereto. Thus, in another aspect of the invention, the structural features of an anti-BMP2, anti-BMP4, anti-BMPR1A, anti-BMPR1B, anti-ACTR1, and/or anti-BMPR2 antibody of the invention are used to create structurally related anti-BMP2, anti-BMP4, anti-BMPR1A, anti-BMPR1B, anti-ACTR1, and/or anti-BMPR2 antibodies that retain at least one functional property of the antibodies of the invention, such as specific binding to human BMP2, BMP4, BMPR1A, BMPR1B, ACTR1, and/or BMPR2. For example, one or more CDR regions of an inventive anti-BMP2, anti-BMP4, anti-BMPR1A, anti-BMPR1B, anti-ACTR1, and/or anti-BMPR2 antibody or mutations thereof, can be combined recombinantly with known framework regions and/or other CDRs to create additional, recombinantly-engineered, antibodies of the present invention, as discussed above.

Other types of modifications include those described in the previous section. The starting material for the engineering method is one or more of the V_{H} and/or V_{K} sequences provided herein or one or more CDR regions thereof. To create the engineered antibody, it is not necessary to actually prepare (i.e. express as a protein) an antibody having one or more of the V_{H} and/or V_{K} sequences provided herein or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Accordingly, in another embodiment, the invention provides a method for preparing an anti-BMP2, anti-BMP4, anti-BMPR1A, anti-BMPR1B, anti-ACTR1, and/or anti-BMPR2 antibody. In a preferred embodiment, the invention provides a method for preparing an anti-BMP2/BMP4 antibody comprising:

(a) providing: (i) a heavy chain variable region antibody sequence comprising a CDR1 sequence selected from the group consisting of SEQ ID NOs:13, 14, and 15, a CDR2 sequence selected from the group consisting of SEQ ID NOs:16, 17, and 18, and/or a CDR3 sequence selected from the group consisting of SEQ ID NOs:19, 20, and 21; and/or (ii) a light chain variable region antibody sequence comprising a CDR1 sequence selected from the group consisting of SEQ ID NOs:22, 23, and 24, a CDR2 sequence selected from the group consisting of SEQ ID NOs:25, 26, and 27, and/or a CDR3 sequence selected from the group consisting of SEQ ID NOs:28, 29, and 30;

(b) altering at least one amino acid residue within the heavy chain variable region antibody sequence and/or the light chain variable region antibody sequence to create at least one altered antibody sequence; and

(c) expressing the altered antibody sequence as a protein.

Standard molecular biology techniques can be used to prepare and express the altered antibody sequence.

Typically, the antibody encoded by the altered antibody sequence(s) is one that retains one, some or all of the functional properties of one or more of the antibodies described herein, which functional properties include, but are not limited to specifically binding to BMP2, BMP4, BMPR1A, BMPR1B, ACTR1, and/or BMPR2.

The functional properties of the altered antibodies can be assessed using standard assays available in the art and/or described herein, such as those set forth in the Examples (e.g., flow cytometry, binding assays).

In certain embodiments of the methods of engineering antibodies of the invention, mutations can be introduced randomly or selectively along all or part of an anti-BMP2, anti-BMP4, anti-BMPR1A, anti-BMPR1B, anti-ACTR1, and/or anti-BMPR2 antibody coding sequence and the resulting modified anti-BMP2, anti-BMP4, anti-BMPR1A, anti-BMPR1B, anti-ACTR1, and/or anti-BMPR2 antibodies can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly or a combination thereof. Alternatively, PCT Publication WO 03/074679 by Lazar et at describes methods of using computational screening methods to optimize physiochemical properties of antibodies.

### Nucleic Acid Molecules Encoding Antibodies of the Invention

Another aspect of the invention pertains to nucleic acid molecules that encode the antibodies of the invention. The nucleic acids may be present in whole cells, in a cell lysate or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. (1987) Current Protocols in Molecular Biology, Greene Publishing and Wiley Interscience, New York. A nucleic acid of the invention can be, for example, DNA or RNA and may or may not contain intronic sequences. In a preferred embodiment, the nucleic acid is a cDNA molecule.

Nucleic acids of the invention can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. Nucleic acids encoding antibodies obtained from an immunoglobulin gene library (e.g., using phage display techniques) can be recovered. Exemplary nucleic acids molecules of the invention are those encoding the VH and VL sequences of the anti-BMP2, anti-BMP4, anti-BMPR1A, anti-BMPR1B, anti-ACTR1, and/or anti-BMPR2 monoclonal antibodies presented herein.

Preferred nucleic acids molecules of the invention are those encoding the V_{H} and V_{L} sequences of the 6H4, 11F2, and 12E3 monoclonal antibodies. DNA sequences encoding the V_{H} sequences of 6H4, 11F2, and 12E3 are shown in SEQ ID NOs:37, 38, and 39, respectively. DNA sequences encoding the V_{L} sequences of 6H4, 11F2, and 12E3 are shown in SEQ ID NOs:40, 41, and 42, respectively.

Other preferred nucleic acids of the invention are nucleic acids having at least 80% sequence identity, such as at least 85%, at least 90%, at least 95%, at least 98% or at least 99% sequence identity, with one of the sequences shown in SEQ ID NO:37, 38, 39, 40, 41, or 42, which nucleic acids encode an antibody of the invention, or an antigen-binding portion thereof.

The percent identity between two nucleic acid sequences is the number of positions in the sequence in which the nucleotide is identical, taking into account the number of gaps and the length of each gap, which need to be introduced for optimal alignment of the two sequences. The comparison of sequences and determination of percent identity between two sequences can be accomplished using a mathematical algorithm, such as the algorithm of Meyers and Miller or the XBLAST program of Altschul described above.

Still further, preferred nucleic acids of the invention comprise one or more CDR-encoding portions of the nucleic acid sequences shown in SEQ ID NO:37, 38, 39, 40, 41, and 42. In this embodiment, the nucleic acid may encode the heavy chain CDR1, CDR2 and/or CDR3 sequence of 6H4, 11F2, and 12E3 or the light chain CDR1, CDR2 and/or CDR3 sequence of 6H4, 11F2, and 12E3.

Nucleic acids which have at least 80%, such as at least 85%, at least 90%, at least 95%, at least 98% or at least 99% sequence identity, with such a CDR-encoding portion of SEQ ID NO:37, 38, 39, 40, 41, or 42 are also preferred nucleic acids of the invention. Such nucleic acids may differ from the corresponding portion of SEQ ID NO:37, 38, 39, 40, 41, or 42 in a non-CDR coding region and/or in a CDR-coding region. Where the difference is in a CDR-coding region, the nucleic acid CDR region encoded by the nucleic acid typically comprises one or more conservative sequence modification as defined herein compared to the corresponding CDR sequence of 6H4, 11F2, and 12E3.

Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to an scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (CH1, CH2, and CH3). The sequences of human heavy chain constant region genes are known in the art (see e.g., Kabat, E. A., et al. Sequences of Proteins of Immunological Interest, (Fifth Edition, U.S. Department of Health and Human Services, NIH Publication No. 91-3242, 1991) and DNA fragments encompassing these regions can be obtained by standard PCR amplification. The heavy chain constant region can be an IgG1, IgG2, IgG3, IgG4, IgA, IgE, IgM or IgD constant region, but most typically is an IgG1 or IgG4 constant region. For a Fab fragment heavy chain gene, the V_{H}-encoding DNA can be operatively linked to another DNA molecule encoding only the heavy chain CH1 constant region.

The isolated DNA encoding the V_{L} region can be converted to a full-length light chain gene (as well as a Fab light chain gene) by operatively linking the V_{L}-encoding DNA to another DNA molecule encoding the light chain constant region, C_{L}. The sequences of human light chain constant region genes are known in the art (see e.g., Kabat, E. A., et al. Sequences of Proteins of Immunological Interest (Fifth Edition, U.S. Department of Health and Human Services, NIH Publication No. 91-3242, 1991) and DNA fragments encompassing these regions can be obtained by standard PCR amplification. The light chain constant region can be a kappa or lambda constant region, but most typically is a kappa constant region.

To create an scFv gene, the V_{H}- and V_{L}-encoding DNA fragments are operatively linked to another fragment encoding a flexible linker, e.g., encoding the amino acid sequence (Gly₄-Ser)₃, such that the V_{H} and V_{L} sequences can be expressed as a contiguous single-chain protein, with the V_{L} and V_{H} regions joined by the flexible linker (see e.g., Bird et al. Science 242:423-426 (1988); Huston et al. Proc. Natl. Acad. Sci. USA 85:5879-5883 (1988); and McCafferty et al., Nature 348:552-554 (1990)).

### Production of Monoclonal Antibodies of the Invention

Monoclonal antibodies (mAbs) of the present invention can be produced by a variety of techniques, including conventional monoclonal antibody methodology e.g., the standard somatic cell hybridization technique of Kohler and Milstein Nature 256:495 (1975). Although somatic cell hybridization procedures are preferred, in principle, other techniques for producing monoclonal antibody can be employed e.g., viral or oncogenic transformation of B lymphocytes.

The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter and U.S. Pat. Nos. 5,530,101; 5,585,089; 5,693,762 and 6,180,370 to Queen et al.).

In a preferred embodiment, the antibodies of the invention are human monoclonal antibodies. Such human monoclonal antibodies directed against BMP2, BMP4, BMPR1A, BMPR1B, ACTR1; and/or BMPR2 can be generated using transgenic or transchromosomic mice carrying parts of the human immune system rather than the mouse system. These transgenic and transchromosomic mice include mice referred to herein as the HuMAb mouse® and KM mouse®, respectively and are collectively referred to herein as “human Ig mice.”

The HuMAb mouse® (Medarex, Inc.) contains human immunoglobulin gene miniloci that encode unrearranged human heavy (μ and γ) and κ light chain immunoglobulin sequences, together with targeted mutations that inactivate the endogenous μ and κ chain loci (see e.g., Lonberg, et al. Nature 368(6474):856-859 (1994)). Accordingly, the mice exhibit reduced expression of mouse IgM or κ and in response to immunization, the introduced human heavy and light chain transgenes undergo class switching and somatic mutation to generate high affinity human IgGκ monoclonal (Lonberg et al., supra; reviewed in Lonberg Handbook of Experimental Pharmacology 113:49-101 (1994); Lonberg and Huszar Intern. Rev. Immunol. 13:65-93 (1995) and Harding and Lonberg Ann. N.Y. Acad. Sci. 764:536-546 (1995)). The preparation and use of HuMab mice and the genomic modifications carried by such mice, is further described in Taylor, et al. Nucleic Acids Research 20:6287-6295 (1992); Chen et al. International Immunology 5:647-656 (1993); Tuaillon et al. Proc. Natl. Acad. Sci. USA 90:3720-3724 (1995); Choi et al. Nature Genetics 4:117-123 (1993); Chen et al. EMBO J. 12:821-830 (1993); Tuaillon et al. J. Immunol. 152:2912-2920 (1994); Taylor et al. International Immunology 6:579-591 (1994); and Fishwild et al. Nature Biotechnology 14:845-851 (1996), the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al.

In a related embodiment, mice carrying portions of human immunoglobulin genes may be immunized. For example, mice may carry only a V region of a human immunoglobulin gene. Immunization of these animals will result in chimeric antibodies.

In another embodiment, human antibodes of the invention can be raised using a mouse that carries human immunoglobulin sequences on transgenes and transchomosomes, such as a mouse that carries a human heavy chain transgene and a human light chain transchromosome. Such mice, referred to herein as the “KM mouse®”, are described in detail in PCT Publication WO 02/43478 to Ishida et al.

Still further, alternative transgenic animal systems expressing human immunoglobulin genes are available in the art and can be used to raise anti-BMP2, anti-BMP4, anti-BMPR1A, anti-BMPR1B, anti -ACTR1, and/or anti-BMPR2 antibodies of the invention. For example, an alternative transgenic system referred to as the Xenomouse (Amgen, Inc., Thousand Oaks, Calif.) can be used; such mice are described in, for example, U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise antibodies of the invention. For example, mice carrying both a human heavy chain transchromosome and a human light chain tranchromosome, referred to as “TC mice” can be used; such mice are described in Tomizuka et al. Proc. Natl. Acad. Sci. USA 97:722-727 (2000). As another example, cows carrying human heavy and light chain transchromosomes have been described in the art (Kuroiwa et al. Nature Biotechnology 20:889-894 (2002)) and can be used to raise anti-BMP2, anti-BMP4, anti-BMPR1A, anti-BMPR1B, anti-ACTR1, and/or anti-BMPR2 antibodies of the invention.

In addition, naked DNA immunization techniques known in the art can be used (with or without purified BMP2, BMP4, BMPR1A, BMPR1B, ACTR1, and/or BMPR2-related protein or BMP2, BMP4, BMPR1A, BMPR1B, ACTR1, and/or BMPR2 expressing cells) to generate an immune response to the encoded immunogen (for review, see Donnelly et al., 1997, Ann. Rev. Immunol. 15: 617-648, the entire contents of which are expressly incorporated herein by reference). Accordingly, the present invention also includes DNA immunization with an BMP2, BMP4, BMPR1A, BMPR1B, ACTR1, and/or BMPR2 gene or portion thereof.

Human monoclonal antibodies of the invention can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

Antibodies according to the present invention may also be produced by the LEX System™ and Plantibodies™ [Biolex, Inc., Pittsboro, N.C.] methodologies wherein the inventive antibody is produced in transgenic plants. See, recently issued U.S. Pat. No. 6,852,319, entitled “Method of Use of Transgenic Plant Expressed

Antibodies.” The LEX System™ couples the natural characteristics of the small green aquatic plant, Lemnaceae, with genetic engineering and protein recovery methods to create a development and production technology that, depending upon the precise application contemplated, may provide certain advantages over existing cell culture and transgenic expression systems. See, U.S. Pat. No. 6,040,498, entitled “Genetically Engineered Duckweed” and PCT Patent Application Publication No. WO 99/07210 (disclosing methods of transformation and selection, methods of transgenic plant regeneration, methods of growth and recovery, use of multiple genes and vectors, and transformed tissue and plants); PCT Patent Application Publication No. WO 02/10414, entitled “Expression of Biologically Active Polypeptides in Duckweed” (disclosing methods and compositions for expression, methods and compositions for recovery, methods for enhanced expression levels, and methods for directed secretion); PCT Patent Application Publication No. WO 02/097029 entitled “Plate and Method for High Throughput Screening”; PCT Patent Application Publication No. WO 02/097433, entitled “Use of Duckweed in High Throughput Screening”; and U.S. Pat. No. 6,680,200, entitled “LED Array for Illuminating Cell Well Plates and Automated Rack System for Handling the Same”. The Plantibodies™ methodology for the expression of human and other antibodies in plants is disclosed in U.S. Pat. Nos. 6,417,429; 5,202,422; 5,639,947; 5,959,177; and 6,417,429. Each of these patents and patent applications is hereby incorporated by reference in its entirety.

### Immunization of Human Ig Mice

When human Ig mice are used to raise human antibodies of the invention, such mice can be immunized with a BMP2, BMP4, BMPR1A, BMPR1B, ACTR1, and/or BMPR2 antibodies of the invention BMP2, BMP4, BMPR1A, BMPR1B, ACTR1, and/or BMPR2 antibodies of the invention BMP2, BMP4, BMPR1A, BMPR1B, ACTR1, and/or BMPR2 antibodies of the invention expressing cell line, a purified or enriched preparation of BMP2, BMP4, BMPR1A, BMPR1B, ACTR1, and/or BMPR2 antigen and/or recombinant BMP2, BMP4, BMPR1A, BMPR1B, ACTR1, and/or BMPR2 or a BMP2, BMP4, BMPR1A, BMPR1B, ACTR1, and/or BMPR2 fusion protein, as described by Lonberg et al. Nature 368(6474):856-859 (1994); Fishwild et al. Nature Biotechnology 14:845-851 (1996); and PCT Publication WO 98/24884 and WO 01/14424. Typically, the mice will be 6-16 weeks of age upon the first immunization. For example, a purified or recombinant preparation (5-50 μg) of antigen can be used to immunize the human Ig mice intraperitoneally.

Detailed procedures to generate fully human monoclonal antibodies to BMP2, BMP4, BMPR1A, BMPR1B, ACTR1, and/or BMPR2 are described in Example 1 below. Cumulative experience with various antigens has shown that the transgenic mice respond when initially immunized intraperitoneally (IP) with antigen in complete Freund's adjuvant, followed by every other week IP immunizations up to a total of 6) with antigen in incomplete Freund's adjuvant. However, adjuvants other than Freund's are also found to be effective. In addition, whole cells in the absence of adjuvant are found to be highly immunogenic. The immune response can be monitored over the course of the immunization protocol with plasma samples being obtained, for example, by retroorbital bleeds. The plasma can be screened by ELISA and mice with sufficient titers of human immunoglobulin can be used for fusions (as described in Example 1). Mice can be boosted intravenously with antigen 3 days before sacrifice and removal of the spleen. It is expected that 2-3 fusions for each immunization may need to be performed. Between 6 and 24 mice are typically immunized for each antigen. Usually, both HCo7 and HCo12 strains are used. Generation of HCo7 and HCo12 mouse strains are described in U.S. Pat. No. 5,770,429 and Example 2 of PCT Publication WO 01/09187, respectively. In addition, both HCo7 and HCo12 transgene can be bred together into a single mouse having two different human heavy chain transgenes (HCo7/HCo12). Alternatively or additionally, the KM mouse® strain can be used, as described in PCT Publication WO 02/43478.

### Generation of Hybridomas Producing Human Monoclonal Antibodies of the Invention

To generate hybridomas producing human monoclonal antibodies of the invention, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3X63-Ag8.653 nonsecreting mouse myeloma cells (ATCC, CRL 1580) with 50% PEG. Alternatively, the single cell suspensions of splenic lymphocytes from immunized mice can be fused using an electric field based electrofusion method, using a Cyto Pulse large chamber cell fusion electroporator (Cyto Pulse Sciences, Inc., Glen Burnie, Md.). Cells are plated at approximately 2×10⁵ in flat bottom microtiter plate, followed by a one week incubation in DMEM high glucose medium with L-glutamine and sodium pyruvate (Mediatech, Inc., Herndon, Va.) and further containing 20% fetal Bovine Serum (Hyclone, Logan, Utah), 18% P388DI conditional media, 5% Origen Hybridoma cloning factor (BioVeris, Gaithersburg, Va.), 4 mM L-glutamine, 5 mM HEPES, 0.055 mM β-mercaptoethanol, 50 units/ml penicillin, 50 mg/ml streptomycin and 1× Hypoxanthine-aminopterin-thymidine (HAT) media (Sigma; the HAT is added 24 hours after the fusion). After one week, cells cultured in medium in which HAT was used was replaced with HT. Individual wells can then be screened by ELISA for human monoclonal IgM and IgG antibodies. Hybridoma growth can be observed usually after 10-14 days. The antibody secreting hybridomas can be re-plated, screened again and if still positive for human IgG, the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS and the concentration can be determined by OD280 using 1.43 extinction coefficient. The monoclonal antibodies can be aliquoted and stored at −80° C.

### Generation of Transfectomas Producing Monoclonal Antibodies of the Invention

Antibodies of the invention also can be produced in a host cell transfectoma using, for example, a combination of recombinant DNA techniques and gene transfection methods as is well known in the art (e.g., Morrison Science 229:1202 (1985)).

For example, to express the antibodies or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector or blunt end ligation if no restriction sites are present).

The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{K} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e. a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe et al., Mol. Cell. Biol. 8:466-472 (1988)).

In addition to the antibody chain genes and regulatory sequences, the recombinant expression vectors of the invention may carry additional sequences, such as sequences that regulate replication of the vector in host cells (e.g. origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells and most typically mammalian host cells, is the most preferred because such eukaryotic cells and in particular mammalian cells, are more likely than prokaryotic cells to assemble and secrete a properly folded and immunologically active antibody. Prokaryotic expression of antibody genes has been reported to be ineffective for production of high yields of active antibody (Boss and Wood, Immunology Today 6:12-13 (1985)).

Preferred mammalian host cells for expressing the recombinant antibodies of the invention include Chinese Hamster Ovary (CHO cells) (including dhfr-CHO cells, described in Urlaub and Chasin, Proc. Natl. Acad. Sci. U.S.A. 77:4216-4220 (1980), used with a DHFR selectable marker, e.g., as described in Kaufman and Sharp Mol. Biol. 159:601-621 (1982)), NSO myeloma cells, COS cells and SP2 cells. In particular, for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more typically, secretion of the antibody into the culture medium in which the host cells are grown. Antibodies can be recovered from the culture medium using standard protein purification methods.

### Characterization of Antibody Binding to Antigen

Antibodies of the invention can be tested for binding to BMP2, BMP4, BMPR1A, BMPR1B, ACTR1, and/or BMPR2 by, for example, flow cytometry. Briefly, BMP2, BMP4, BMPR1A, BMPR1B, ACTR1, and/or BMPR2 expressing cells are freshly harvested from tissue culture flasks and a single cell suspension prepared. BMP2, BMP4, BMPR1A, BMPR1B, ACTR1, and/or BMPR2 expressing cell suspensions are either stained with primary antibody directly or after fixation with 1% paraformaldehyde in PBS with or without permeabilization. Approximately one million cells are resuspended in PBS containing 0.5% BSA and 50-200 μg/ml of primary antibody and incubated on ice for 30 minutes. The cells are washed twice with PBS containing 0.1% BSA, 0.01% NaN₃, resuspended in 100 μl of 1:100 diluted F1TC-conjugated goat-anti-human IgG (Jackson ImmunoResearch, West Grove, Pa.) and incubated on ice for an additional 30 minutes. The cells are again washed twice, resuspended in 0.5 ml of wash buffer and analyzed for fluorescent staining on a FACSCalibur cytometer (Becton-Dickinson, San Jose, Calif.).

Alternatively, antibodies of the invention can be tested for binding to BMP2, BMP4, BMPR1A, BMPR1B, ACTR1, and/or BMPR2 by standard ELISA. Briefly, microtiter plates are coated with purified BMP2, BMP4, BMPR1A, BMPR1B, ACTR1, and/or BMPR2 at 0.25 μg/ml in PBS and then blocked with 5% bovine serum albumin in PBS. Dilutions of antibody (e.g., dilutions of plasma from BMP2, BMP4, BMPR1A, BMPR1B, ACTR1, and/or BMPR2 immunized mice) are added to each well and incubated for 1-2 hours at 37° C. The plates are washed with PBS/Tween and then incubated with secondary reagent (e.g., for human antibodies, a goat-anti-human IgG Fc-specific polyclonal reagent) conjugated to alkaline phosphatase for 1 hour at 37° C. After washing, the plates are developed with pNPP substrate (1 mg/ml) and analyzed at OD of 405-650. Typically, mice which develop the highest titers will be used for fusions.

An ELISA assay as described above can also be used to screen for hybridomas that show positive reactivity with BMP2, BMP4, BMPR1A, BMPR1B, ACTR1, and/or BMPR2 immunogen. Hybridomas that bind with high avidity to BMP2, BMP4, BMPR1A, BMPR1B, ACTR1, and/or BMPR2 are subcloned and further characterized. One clone from each hybridoma, which retains the reactivity of the parent cells (by ELISA), can be chosen for making a 5-10 vial cell bank stored at -140° C. and for antibody purification.

To purify anti-BMP2, anti-BMP4, anti-BMPR1A, anti-BMPR1B, anti-ACTR1, and/or anti-BMPR2 antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS and the concentration can be determined by OD₂₈₀ using 1.43 extinction coefficient. The monoclonal antibodies can be aliquoted and stored at −80° C.

To determine if the selected anti-BMP2, anti-BMP4, anti-BMPR1A, anti-BMPR1B, anti-ACTR1, and/or anti-BMPR2 monoclonal antibodies bind to unique epitopes, each antibody can be biotinylated using commercially available reagents (Pierce, Rockford, Ill.). Competition studies using unlabeled monoclonal antibodies and biotinylated monoclonal antibodies can be performed using BMP2, BMP4, BMPR1A, BMPR1B, ACTR1, and/or BMPR2 coated-ELISA plates as described above. Biotinylated mAb binding can be detected with a strep-avidin-alkaline phosphatase probe. Alternatively, competition studies can be performed using radiolabelled antibody and unlabelled competing antibodies can be detected in a Scatchard analysis, as further described in the Examples below.

To determine the isotype of purified antibodies, isotype ELISAs can be performed using reagents specific for antibodies of a particular isotype. For example, to determine the isotype of a human monoclonal antibody, wells of microtiter plates can be coated with 1 μg/ml of anti-human immunoglobulin overnight at 4° C. After blocking with 1% BSA, the plates are reacted with 1 μg/ml or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

Anti-BMP2, anti-BMP4, anti-BMPR1A, anti-BMPR1B, anti-ACTR1, and/or anti-BMPR2 human IgGs can be further tested for reactivity with BMP2, BMP4, BMPR1A, BMPR1B, ACTR1, and/or BMPR2 antigen by Western blotting. Briefly, BMP2, BMP4, BMPR1A, BMPR1B, ACTR1, and/or BMPR2 can be prepared and subjected to sodium dodecyl sulfate polyacrylamide gel electrophoresis. After electrophoresis, the separated antigens are transferred to nitrocellulose membranes, blocked with 10% fetal calf serum and probed with the monoclonal antibodies to be tested. Human IgG binding can be detected using anti-human IgG alkaline phosphatase and developed with BCIP/NBT substrate tablets (Sigma Chem. Co., St. Louis, Mo.).

### Immunoconjugates

In another aspect, the present invention features anti-BMP2, anti-BMP4, anti-BMPR1A, anti-BMPR1B, anti-ACTR1, and/or anti-BMPR2 antibodies, or a fragments thereof, conjugated to a therapeutic moiety, such as a cytotoxin, a drug (e.g., an immunosuppressant) or a radiotoxin. Such conjugates are referred to herein as “immunoconjugates”. Immunoconjugates that include one or more cytotoxins are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thiotepa, chlorambucil, melphalan, carmustine (BSNU) and lomustine (CCNU), cyclothosphamide, busulfan, dibromomannitol, streptozotocin, mitomycin C and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin and anthramycin (AMC)) and anti-mitotic agents (e.g., vincristine and vinblastine).

Within certain aspects of the present invention are provided immunoconjugates comprising an anti-BMP2, anti-BMP4, anti-BMPR1A, anti-BMPR1B, anti-ACTR1, and/or anti-BMPR2 antibodies, or a fragments thereof, conjugated to a therapeutic moiety wherein the therapeutic moiety is an Ultra-potent Therapeutic (UPT™; Medarex, Inc., Milpitas, Calif.) as disclosed in U.S. Pat. Nos. 6,1003,236 and 6,638,509 [describing a toxin conjugate wherein the toxin (e.g., vinblastine, risin, diphtheria toxin, abrin, vinblastine hydrazide, methotrexate hydrazide, anthracycline, chelates of indium and yttrium, metal chelates, and anthracycline) is bound via a cleavable spacer comprising polyethylene glycol and dipeptide to a residue, such as on an antibody or fragment thereof]; U.S. Pat. No. 6,989,452 and U.S. patent application Ser. Nos. 10/160,972, 10/161,234, 11/133,970, 11/134,685, 11/134,826, 11/224,580, and 11/398,854 [describing cytotoxic agents, disulfide prodrugs, peptidyl prodrugs, and linkers, including chemical linkers].

Other preferred examples of therapeutic cytotoxins that can be conjugated to an antibody of the invention include duocarmycins, calicheamicins, maytansines and auristatins and derivatives thereof. An example of a calicheamicin antibody conjugate is commercially available (Mylotarg®; American Home Products).

Cytotoxins can be conjugated to antibodies of the invention using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D).

Examples of cytotoxins are described, for example, in U.S. Pat. Nos. 6,989,452, 7,087,600, and 7,129,261, and in PCT Application Nos. PCT/US02/17210, PCT/US2005/017804, PCT/US06/37793, PCT/US06/060050, PCT/US2006/060711, WO/2006/110476, and in U.S. Patent Application No. 60/891,028, all of which are incorporated herein by reference in their entirety. For further discussion of types of cytotoxins, linkers and methods for conjugating therapeutic agents to antibodies, see also Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264.

Antibodies of the present invention also can be conjugated to a radioactive isotope to generate cytotoxic radiopharmaceuticals, also referred to as radioimmunoconjugates. Examples of radioactive isotopes that can be conjugated to antibodies for use diagnostically or therapeutically include, but are not limited to, iodine¹³¹, iodine¹²⁵, indium¹¹¹, yttrium⁹⁰ and lutetium¹⁷⁷. Method for preparing radioimmunconjugates are established in the art. Examples of radioimmunoconjugates are commercially available, including Zevalin™ (Biogen® IDEC) and Bexxar™ (Glaxo-SmithKline) and ®(Corixa Pharmaceuticals), and similar methods can be used to prepare radioimmunoconjugates using the antibodies of the invention.

The antibody conjugates of the invention can be used to modify a given biological response and the drug moiety is not to be construed as limited to classical chemical therapeutic agents. For example, the drug moiety may be a protein or polypeptide possessing a desired biological activity. Such proteins may include, for example, an enzymatically active toxin or active fragment thereof, such as abrin, ricin A, pseudomonas exotoxin or diphtheria toxin; a protein such as tumor necrosis factor or interferon-γ; or, biological response modifiers such as, for example, lymphokines, inter leukin-1 (”IL-1”), interleukin-2 (“IL-2”), interleukin-6 (“IL-6”), granulocyte macrophage colony stimulating factor (“GM-CSF”), granulocyte colony stimulating factor (“G-CSF”) or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy pp. 243-56 (Reisfeld et al., eds., Alan R. Liss, Inc. 1985); Hellstrom et al., “Antibodies For Drug Delivery”, in Controlled Drug Delivery pp. 623-53 (2nd Ed., Robinson et al., eds., Marcel Dekker, Inc. 1987); Thorpe, “Antibody Carriers Of Cytotoxic Agents In Cancer Therapy: A Review”, in Monoclonal Antibodies '84: Biological and Clinical Applications, pp. 475-506 (1985); “Analysis, Results and Future Prospective Of The Therapeutic Use Of Radiolabeled Antibody In Cancer Therapy”, in Monoclonal Antibodies For Cancer Detection And Therapy, pp. 303-16 (Academic Press 1985); and Thorpe et al., “The Preparation And Cytotoxic Properties Of Antibody-Toxin Conjugates”, Immunol. Rev. 62:119-58 (1982).

### Bispecific Molecules

In another aspect, the present invention features bispecific molecules comprising an anti-BMP2, anti-BMP4, anti-BMPR1A, anti-BMPR1B, anti-ACTR1, and/or anti-BMPR2 antibodies or fragments thereof, of the present invention. Such an antibody or antigen-binding portion thereof, can be derivatized or linked to another functional molecule, e.g., another peptide or protein (e.g., another antibody or ligand for a receptor) to generate a bispecific molecule that binds to at least two different binding sites or target molecules. The antibody of the invention may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule of the invention, an antibody of the invention can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present invention includes bispecific molecules comprising at least one first binding specificity for BMP2, BMP4, BMPR1A, BMPR1B, ACTR1, and/or BMPR2 and a second binding specificity for a second target epitope. In a particular embodiment of the invention, the second target epitope is an Fc receptor, e.g., human FcγRI (CD64) or a human Fcα receptor (CD89). Therefore, the invention includes bispecific molecules capable of binding both to FcγR or FcαR expressing effector cells (e.g., monocytes, macrophages or polymorphonuclear cells (PMNs)) and to target cells expressing BMP2, BMP4, BMPR1A, BMPR1B, ACTR1, and/or BMPR2. These bispecific molecules target BMP2, BMP4, BMPR1A, BMPR1B, ACTR1, and/or BMPR2 expressing cells to effector cell and trigger Fc receptor-mediated effector cell activities, such as phagocytosis of a BMP2, BMP4, BMPR1A, BMPR1B, ACTR1, and/or BMPR2 expressing cell, antibody dependent cell-mediated cytotoxicity (ADCC), cytokine release or generation of superoxide anion.

In an embodiment of the invention in which the bispecific molecule is multispecific, the molecule can further include a third binding specificity, in addition to an anti-Fc binding specificity and an anti-BMP2, anti-BMP4, anti-BMPR1A, anti-BMPR1B, anti-ACTR1, and/or anti-BMPR2 binding specificity. In one embodiment, the third binding specificity is an anti-enhancement factor (EF) portion, e.g., a molecule which binds to a surface protein involved in cytotoxic activity and thereby increases the immune response against the target cell. The “anti-enhancement factor portion” can be an antibody, functional antibody fragment or a ligand that binds to a given molecule, e.g., an antigen or a receptor and thereby results in an enhancement of the effect of the binding determinants for the F_{c} receptor or target cell antigen. The “anti-enhancement factor portion” can bind an F_{c} receptor or a target cell antigen. Alternatively, the anti-enhancement factor portion can bind to an entity that is different from the entity to which the first and second binding specificities bind. For example, the anti-enhancement factor portion can bind a cytotoxic T-cell (e.g., via CD2, CD3, CD8, CD28, CD4, CD40, ICAM-1 or other immune cell that results in an increased immune response against the target cell).

In one embodiment, the bispecific molecules of the invention comprise as a binding specificity at least one antibody or an antibody fragment thereof, including, e.g., an Fab, Fab′, F(ab′)₂, Fv, Fd, dAb, or a single chain Fv. The antibody may also be a light chain or heavy chain dimer or any minimal fragment thereof such as a Fv or a single chain construct as described in Ladner et al. U.S. Pat. No. 4,946,778 to Ladner et al., the contents of which is expressly incorporated by reference.

In another embodiment, the binding specificity for an Fcγ receptor is provided by a monoclonal antibody, the binding of which is not blocked by human immunoglobulin G (IgG). As used herein, the term “IgG receptor” refers to any of the eight γ-chain genes located on chromosome 1. These genes encode a total of twelve transmembrane or soluble receptor isoforms which are grouped into three Fcγ receptor classes: FcγRI (CD64), FcγRII(CD32) and FcγRIII (CD16). In one preferred embodiment, the Fcγ receptor a human high affinity FcγRI. The human FcγRI is a 72 kDa molecule, which shows high affinity for monomeric IgG (10⁸-10⁹ M⁻¹).

The production and characterization of certain anti-Fcγ monoclonal antibodies are described by Fanger et al. in PCT Publication WO 88/00052 and in U.S. Pat. No. 4,954,617 to Fanger et al., the teachings of which are fully incorporated by reference herein. These antibodies bind to an epitope of FcγRI, FcγRII or FcγRIII at a site which is distinct from the Fcγ binding site of the receptor and, thus, their binding is not blocked substantially by physiological levels of IgG. Specific anti-FcγRI antibodies useful in this invention are mAb 22, mAb 32, mAb 44, mAb 62 and mAb 197. The hybridoma producing mAb 32 is available from the American type Culture Collection, ATCC Accession No. HB9469. In other embodiments, the anti-Fcγ receptor antibody is a humanized form of monoclonal antibody 22 (H22). The production and characterization of the H22 antibody is described in Graziano et al. J. Immunol 155 (10):4996-5002 (1995) and PCT Publication WO 94/10332 to Tempest et al. The H22 antibody producing cell line was deposited at the American type Culture Collection under the designation HA022CL1 and has the Accession No. CRL 11177.

In still other embodiments, the binding specificity for an Fc receptor is provided by an antibody that binds to a human IgA receptor, e.g., an Fc-alpha receptor (FcαRI (CD89)), the binding of which is typically not blocked by human immunoglobulin A (IgA). The term “IgA receptor” is intended to include the gene product of one α-gene (FcαRI) located on chromosome 19. This gene is known to encode several alternatively spliced transmembrane isoforms of 55 to 110 kDa. Fcα RI (CD89) is constitutively expressed on monocytes/macrophages, eosinophilic and neutrophilic granulocytes, but not on non-effector cell populations. FcαRI has medium affinity (≈5×10⁷ M⁻¹) for both IgA1 and IgA2, which is increased upon exposure to cytokines such as G-CSF or GM-CSF (Morton et al., Critical Reviews in Immunology 16:423-440 (1996)). Four FcαRI-specific monoclonal antibodies, identified as A3, A59, A62 and A77, which bind FcαRI outside the IgA ligand binding domain, have been described (Monteiro et al., J. Immunol. 148:1764 (1992)).

FcαRI and FcγRI are preferred trigger receptors for use in the bispecific molecules of the invention because they are (1) expressed primarily on immune effector cells, e.g., monocytes, PMNs, macrophages and dendritic cells; (2) expressed at high levels (e.g., 5,000-100,000 per cell); (3) mediators of cytotoxic activities (e.g., ADCC, phagocytosis); and (4) mediate enhanced antigen presentation of antigens, including self-antigens, targeted to them.

Bispecific molecules of the present invention can be prepared by conjugating the constituent binding specificities, e.g., the anti-FcR and anti-BMP2, anti-BMP4, anti-BMPR1A, anti-BMPR1B, anti-ACTR1, and/or anti-BMPR2 binding specificities, using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP) and sulfosuccinimidyl 4-(N-maleimidomethyl)cyclohaxane-1-carboxylate (sulfo-SMCC) (see e.g., Karpovsky et al. J. Exp. Med. 160:1686 (1984); Liu et al. Proc. Natl. Acad. Sci. U.S.A. 82:8648 (1985)). Other methods include those described in Paulus, Behring Ins. Mitt. No. 78:118-132 (1985); Brennan et al. Science 229:81-83 (1985)) and Glennie et al. J. Immunol. 139:2367-2375 (1987)). Preferred conjugating agents are SATA and sulfo-SMCC, both available from Pierce Chemical Co. (Rockford, Ill.).

When the binding specificities are antibodies, they can be conjugated via sulfhydryl bonding of the C-terminus hinge regions of the two heavy chains. In a particularly preferred embodiment, the hinge region is modified to contain an odd number of sulfhydryl residues, typically one, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)₂ or ligand×Fab fusion protein. A bispecific molecule of the invention can be a single chain molecule comprising one single chain antibody and a binding determinant or a single chain bispecific molecule comprising two binding determinants. Bispecific molecules may comprise at least two single chain molecules. Methods for preparing bispecific molecules are described for example in U.S. Pat. Nos. 5,260,203; 5,455,030; 4,881,175; 5,132,405; 5,091,513; 5,476,786; 5,013,653; 5,258,498; and 5,482,858, each of which is incorporated by reference herein in its entirety.

Binding of the bispecific molecules to their specific targets can be confirmed by, for example, enzyme-linked immunosorbent assay (ELISA), radioimmunoassay (RIA), FACS analysis, bioassay (e.g., growth inhibition) or Western Blot assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography.

### Antibody Fragments and Antibody Mimetics

The instant invention is not limited to traditional antibodies and may be practiced through the use of antibody fragments and antibody mimetics. As detailed below, a wide variety of antibody fragment and antibody mimetic technologies have now been developed and are widely known in the art. While a number of these technologies, such as domain antibodies, Nanobodies, and UniBodies make use of fragments of, or other modifications to, traditional antibody structures, there are also alternative technologies, such as Affibodies, DARPins, Anticalins, Avimers, and Versabodies that employ binding structures that, while they mimic traditional antibody binding, are generated from and function via distinct mechanisms.

Domain Antibodies (dAbs) are the smallest functional binding units of antibodies, corresponding to the variable regions of either the heavy (VH) or light (VL) chains of human antibodies. Domain Antibodies have a molecular weight of approximately 13 kDa. Domantis Limited has developed a series of large and highly functional libraries of fully human VH and VL dAbs (more than ten billion different sequences in each library), and uses these libraries to select dAbs that are specific to therapeutic targets. In contrast to many conventional antibodies, Domain Antibodies are well expressed in bacterial, yeast, and mammalian cell systems. Further details of domain antibodies and methods of production thereof may be obtained by reference to U.S. Pat. Nos. 6,291,158; 6,582,915; 6,593,081; 6,172,197; 6,696,245; US Serial No. 2004/0110941; European patent application No. 1433846 and European Patents 0368684 & 0616640; WO05/035572, WO04/101790, WO04/081026, WO04/058821, WO04/003019 and WO03/002609, each of which is incorporated herein by reference in its entirety.

Nanobodies are antibody-derived therapeutic proteins that contain the unique structural and functional properties of naturally-occurring heavy-chain antibodies. These heavy-chain antibodies contain a single variable domain (VHH) and two constant domains (CH2 and CH3). Importantly, the cloned and isolated VHH domain is a perfectly stable polypeptide harboring the full antigen-binding capacity of the original heavy-chain antibody. Nanobodies have a high homology with the VH domains of human antibodies and can be further humanized without any loss of activity. Importantly, Nanobodies have a low immunogenic potential, which has been confirmed in primate studies with Nanobody lead compounds.

Nanobodies combine the advantages of conventional antibodies with important features of small molecule drugs. Like conventional antibodies, Nanobodies show high target specificity, high affinity for their target and low inherent toxicity. However, like small molecule drugs they can inhibit enzymes and readily access receptor clefts. Furthermore, Nanobodies are extremely stable, can be administered by means other than injection (see, e.g., WO 04/041867, which is herein incorporated by reference in its entirety) and are easy to manufacture. Other advantages of Nanobodies include recognizing uncommon or hidden epitopes as a result of their small size, binding into cavities or active sites of protein targets with high affinity and selectivity due to their unique 3-dimensional, drug format flexibility, tailoring of half-life and ease and speed of drug discovery.

Nanobodies are encoded by single genes and are efficiently produced in almost all prokaryotic and eukaryotic hosts e.g., E. coli (see e.g. U.S. Pat. No. 6,765,087, which is herein incorporated by reference in its entirety), molds (for example Aspergillus or Trichoderma) and yeast (for example Saccharomyces, Kluyveromyces, Hansenula or Pichia) (see, e.g., U.S. Pat. No. 6,838,254, which is herein incorporated by reference in its entirety). The production process is scalable and multi-kilogram quantities of Nanobodies have been produced. Because Nanobodies exhibit a superior stability compared with conventional antibodies, they can be formulated as a long shelf-life, ready-to-use solution.

The Nanoclone method (see e.g., WO 06/079372, which is herein incorporated by reference in its entirety) is a proprietary method for generating Nanobodies against a desired target, based on automated high-throughout selection of B-cells and could be used in the context of the instant invention.

UniBodies are another antibody fragment technology, however this one is based upon the removal of the hinge region of IgG4 antibodies. The deletion of the hinge region results in a molecule that is essentially half the size of traditional IgG4 antibodies and has a univalent binding region rather than the bivalent binding region of IgG4 antibodies. It is also well known that IgG4 antibodies are inert and thus do not interact with the immune system, which may be advantageous for the treatment of diseases where an immune response is not desired, and this advantage is passed onto UniBodies. For example, UniBodies may function to inhibit or silence, but not kill, the cells to which they are bound. Additionally, UniBody binding to cancer cells do not stimulate them to proliferate. Furthermore, because UniBodies are about half the size of traditional IgG4 antibodies, they may show better distribution over larger solid tumors with potentially advantageous efficacy. UniBodies are cleared from the body at a similar rate to whole IgG4 antibodies and are able to bind with a similar affinity for their antigens as whole antibodies. Further details of UniBodies may be obtained by reference to PCT Publication No. WO2007/059782, which is herein incorporated by reference in its entirety.

Affibody molecules represent a new class of affinity proteins based on a 58-amino acid residue protein domain, derived from one of the IgG-binding domains of staphylococcal protein A. This three helix bundle domain has been used as a scaffold for the construction of combinatorial phagemid libraries, from which Affibody variants that target the desired molecules can be selected using phage display technology (Nord K, Gunneriusson E, Ringdahl J, Stahl S, Uhlen M, Nygren P A, Binding proteins selected from combinatorial libraries of an α-helical bacterial receptor domain, Nat Biotechnol 1997; 15:772-7. Ronmark J, Gronlund H, Uhlen M, Nygren P A, Human immunoglobulin A (IgA)-specific ligands from combinatorial engineering of protein A, Eur J Biochem 2002; 269:2647-55.). The simple, robust structure of Affibody molecules in combination with their low molecular weight (6 kDa), make them suitable for a wide variety of applications, for instance, as detection reagents (Ronmark J, Hansson M, Nguyen T, et al, Construction and characterization of affibody-Fc chimeras produced in Escherichia coli, J Immunol Methods 2002; 261:199-211) and to inhibit receptor interactions (Sandstorm K, Xu Z, Forsberg G, Nygren P A, Inhibition of the CD28-CD80 co-stimulation signal by a CD28-binding Affibody ligand developed by combinatorial protein engineering, Protein Eng 2003; 16:691-7). Further details of Affibodies and methods of production thereof may be obtained by reference to U.S. Pat. No 5,831,012 which is herein incorporated by reference in its entirety.

Labeled Affibodies may also be useful in imaging applications for determining abundance of Isoforms.

DARPins (Designed Ankyrin Repeat Proteins) are one example of an antibody mimetic DRP (Designed Repeat Protein) technology that has been developed to exploit the binding abilities of non-antibody polypeptides. Repeat proteins such as ankyrin or leucine-rich repeat proteins, are ubiquitous binding molecules, which occur, unlike antibodies, intra- and extracellularly. Their unique modular architecture features repeating structural units (repeats), which stack together to form elongated repeat domains displaying variable and modular target-binding surfaces. Based on this modularity, combinatorial libraries of polypeptides with highly diversified binding specificities can be generated. This strategy includes the consensus design of self-compatible repeats displaying variable surface residues and their random assembly into repeat domains.

DARPins can be produced in bacterial expression systems at very high yields and they belong to the most stable proteins known. Highly specific, high-affinity DARPins to a broad range of target proteins, including human receptors, cytokines, kinases, human proteases, viruses and membrane proteins, have been selected. DARPins having affinities in the single-digit nanomolar to picomolar range can be obtained.

DARPins have been used in a wide range of applications, including ELISA, sandwich ELISA, flow cytometric analysis (FACS), immunohistochemistry (IHC), chip applications, affinity purification or Western blotting. DARPins also proved to be highly active in the intracellular compartment for example as intracellular marker proteins fused to green fluorescent protein (GFP). DARPins were further used to inhibit viral entry with IC50 in the pM range. DARPins are not only ideal to block protein-protein interactions, but also to inhibit enzymes. Proteases, kinases and transporters have been successfully inhibited, most often an allosteric inhibition mode. Very fast and specific enrichments on the tumor and very favorable tumor to blood ratios make DARPins well suited for in vivo diagnostics or therapeutic approaches.

Additional information regarding DARPins and other DRP technologies can be found in US Patent Application Publication No. 2004/0132028 and International Patent Application Publication No. WO 02/20565, both of which are hereby incorporated by reference in their entirety.

Anticalins are an additional antibody mimetic technology, however in this case the binding specificity is derived from lipocalins, a family of low molecular weight proteins that are naturally and abundantly expressed in human tissues and body fluids. Lipocalins have evolved to perform a range of functions in vivo associated with the physiological transport and storage of chemically sensitive or insoluble compounds. Lipocalins have a robust intrinsic structure comprising a highly conserved β-barrel which supports four loops at one terminus of the protein. These loops form the entrance to a binding pocket and conformational differences in this part of the molecule account for the variation in binding specificity between individual lipocalins.

While the overall structure of hypervariable loops supported by a conserved β-sheet framework is reminiscent of immunoglobulins, lipocalins differ considerably from antibodies in terms of size, being composed of a single polypeptide chain of 160-180 amino acids which is marginally larger than a single immunoglobulin domain.

Lipocalins are cloned and their loops are subjected to engineering in order to create Anticalins. Libraries of structurally diverse Anticalins have been generated and Anticalin display allows the selection and screening of binding function, followed by the expression and production of soluble protein for further analysis in prokaryotic or eukaryotic systems. Studies have successfully demonstrated that Anticalins can be developed that are specific for virtually any human target protein can be isolated and binding affinities in the nanomolar or higher range can be obtained.

Anticalins can also be formatted as dual targeting proteins, so-called Duocalins. A Duocalin binds two separate therapeutic targets in one easily produced monomeric protein using standard manufacturing processes while retaining target specificity and affinity regardless of the structural orientation of its two binding domains.

Modulation of multiple targets through a single molecule is particularly advantageous in diseases known to involve more than a single causative factor. Moreover, bi- or multivalent binding formats such as Duocalins have significant potential in targeting cell surface molecules in disease, mediating agonistic effects on signal transduction pathways or inducing enhanced internalization effects via binding and clustering of cell surface receptors. Furthermore, the high intrinsic stability of Duocalins is comparable to monomeric Anticalins, offering flexible formulation and delivery potential for Duocalins.

Additional information regarding Anticalins can be found in U.S. Pat. No. 7,250,297 and International Patent Application Publication No. WO 99/16873, both of which are hereby incorporated by reference in their entirety.

Another antibody mimetic technology useful in the context of the instant invention are Avimers. Avimers are evolved from a large family of human extracellular receptor domains by in vitro exon shuffling and phage display, generating multidomain proteins with binding and inhibitory properties. Linking multiple independent binding domains has been shown to create avidity and results in improved affinity and specificity compared with conventional single-epitope binding proteins. Other potential advantages include simple and efficient production of multitarget-specific molecules in Escherichia coli, improved thermostability and resistance to proteases. Avimers with sub-nanomolar affinities have been obtained against a variety of targets.

Additional information regarding Avimers can be found in US Patent Application Publication Nos. 2006/0286603, 2006/0234299, 2006/0223114, 2006/0177831, 2006/0008844, 2005/0221384, 2005/0164301, 2005/0089932, 2005/0053973, 2005/0048512, 2004/0175756, all of which are hereby incorporated by reference in their entirety.

Versabodies are another antibody mimetic technology that may be used in the context of the instant invention. Versabodies are small proteins of 3-5 kDa with >15% cysteines, which form a high disulfide density scaffold, replacing the hydrophobic core that typical proteins have. The replacement of a large number of hydrophobic amino acids, comprising the hydrophobic core, with a small number of disulfides results in a protein that is smaller, more hydrophilic (less aggregation and non-specific binding), more resistant to proteases and heat, and has a lower density of T-cell epitopes, because the residues that contribute most to MHC presentation are hydrophobic. All four of these properties are well-known to affect immunogenicity, and together they are expected to cause a large decrease in immunogenicity.

The inspiration for Versabodies comes from the natural injectable biopharmaceuticals produced by leeches, snakes, spiders, scorpions, snails, and anemones, which are known to exhibit unexpectedly low immunogenicity. Starting with selected natural protein families, by design and by screening the size, hydrophobicity, proteolytic antigen processing, and epitope density are minimized to levels far below the average for natural injectable proteins.

Given the structure of Versabodies, these antibody mimetics offer a versatile format that includes multi-valency, multi-specificity, a diversity of half-life mechanisms, tissue targeting modules and the absence of the antibody Fc region. Furthermore, Versabodies are manufactured in E. coli at high yields, and because of their hydrophilicity and small size, Versabodies are highly soluble and can be formulated to high concentrations. Versabodies are exceptionally heat stable (they can be boiled) and offer extended shelf-life.

Additional information regarding Versabodies can be found in US Patent Application Publication No. 2007/0191272 which is hereby incorporated by reference in its entirety.

The detailed description of antibody fragment and antibody mimetic technologies provided above is not intended to be a comprehensive list of all technologies that could be used in the context of the instant specification. For example, and also not by way of limitation, a variety of additional technologies including alternative polypeptide-based technologies, such as fusions of complimentary determining regions as outlined in Qui et al., Nature Biotechnology, 25(8) 921-929 (2007), which is hereby incorporated by reference in its entirety, as well as nucleic acid-based technologies, such as the RNA aptamer technologies described in U.S. Pat. Nos. 5,789,157, 5,864,026, 5,712,375, 5,763,566, 6,013,443, 6,376,474, 6,613,526, 6,114,120, 6,261,774, and 6,387,620, all of which are hereby incorporated by reference, could be used in the context of the instant invention.

### Pharmaceutical Compositions

In another aspect, the present invention provides a composition, e.g., a pharmaceutical composition, containing one or a combination of monoclonal antibodies or antigen-binding portion(s) thereof, of the present invention, formulated together with a pharmaceutically acceptable carrier. Such compositions may include one or a combination of (e.g., two or more different) antibodies or immunoconjugates or bispecific molecules of the invention. For example, a pharmaceutical composition of the invention can comprise a combination of antibodies (or immunoconjugates or bispecifics) that bind to different epitopes on the target antigen or that have complementary activities.

Pharmaceutical compositions of the invention also can be administered in combination therapy, i.e. combined with other agents. For example, the combination therapy can include an anti-BMP2, anti-BMP4, anti-BMPR1A, anti-BMPR1B, anti-ACTR1, and/or anti-BMPR2 antibody of the present invention combined with at least one other anti-inflammatory or immunosuppressant agent, one or more other antibody having efficacy against a bone disease or cancer, and/or one or more chemotherapeutic modality. It will be appreciated that a wide variety of co-therapeutic approaches are contemplated with the advantage that decreased dosages of an inventive anti-BMP2, anti-BMP4, anti-BMPR1A, anti-BMPR1B, anti-ACTR1, and/or anti-BMPR2 antibody may result in a reduction of therapeutic side effects.

Within other embodiments, therapeutic antibodies disclosed herein may be used in combination with one or more antibody that suppresses an immunosuppressive pathway such as, for example, in combination with an anti-CTLA-4 antibody (exemplified herein by the antibody designated MDX-010). The CTLA-4 protein is found on certain lymphocytes that, when recognizing a foreign substance such as a virus or bacteria, initiate an immune response to fight the infection. CTLA-4 proteins help stop the immune response by decreasing the number of immune cells fighting against the virus or bacteria. When an immune response is mounted against bone and/or tumor cells, however, it may be beneficial not to stop the immune response, but instead, to keep a large number of lymphocytes available. Thus, an anti-CTLA-4 antibody, such as MDX-010 may be advantageously used in combination with one or more anti-BMP2, anti-BMP4, anti-BMPR1A, anti-BMPR1B, anti-ACTR1, and/or anti-BMPR2 antibody of the present invention to block CTLA-4 and maintain immune activity.

As used herein, “pharmaceutically acceptable carrier” includes any and all solvents, dispersion media, coatings, antibacterial and antifungal agents, isotonic and absorption delaying agents and the like that are physiologically compatible. Typically, the carrier is suitable for intravenous, intramuscular, subcutaneous, parenteral, spinal or epidermal administration (e.g., by injection or infusion). Depending on the route of administration, the active compound, i.e. antibody, immunoconjugate or bispecific molecule, may be coated in a material to protect the compound from the action of acids and other natural conditions that may inactivate the compound.

The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge et al., J. Pharm. Sci. 66:1-19 (1977)). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol and the like; and (3) metal chelating agents, such as citric acid, ethylenediamine tetraacetic acid (EDTA), sorbitol, tartaric acid, phosphoric acid and the like.

Examples of suitable aqueous and nonaqueous carriers that may be employed in the pharmaceutical compositions of the invention include water, ethanol, polyols (such as glycerol, propylene glycol, polyethylene glycol and the like) and suitable mixtures thereof, vegetable oils, such as olive oil and injectable organic esters, such as ethyl oleate. Proper fluidity can be maintained, for example, by the use of coating materials, such as lecithin, by the maintenance of the required particle size in the case of dispersions and by the use of surfactants.

These compositions may also contain adjuvants such as preservatives, wetting agents, emulsifying agents and dispersing agents. Prevention of presence of microorganisms may be ensured both by sterilization procedures, supra and by the inclusion of various antibacterial and antifungal agents, for example, paraben, chlorobutanol, phenol sorbic acid and the like. It may also be desirable to include isotonic agents, such as sugars, sodium chloride and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome or other ordered structure suitable to high drug concentration. The carrier can be a solvent or dispersion medium containing, for example, water, ethanol, polyol (for example, glycerol, propylene glycol and liquid polyethylene glycol and the like) and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, typically from about 0.1 per cent to about 70 percent, most typically from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved and (b) the limitations inherent in the art of compounding such an active compound for the treatment of sensitivity in individuals.

For administration of the antibody, the dosage ranges from about 0.0001 to 100 mg/kg and more usually 0.01 to 25 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. Higher dosages, e.g., 15 mg/kg body weight, 20 mg/kg body weight or 25 mg/kg body weight can be used as needed. An exemplary treatment regime entails administration once per week, once every two weeks, once every three weeks, once every four weeks, once a month, once every 3 months or once every three to 6 months. Particular dosage regimens for an antibody of the invention include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks.

In some methods, two or more anti-BMP2, anti-BMP4, anti-BMPRIA, anti-BMPR1B, anti-ACTR1, and/or anti-BMPR2 monoclonal antibodies of the invention with different binding specificities are administered simultaneously, in which case the dosage of each antibody administered falls within the ranges indicated. Antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml.

In other methods, one or more anti-BMP2, anti-BMP4, anti-BMPR1A, anti-BMPR1B, anti-ACTR1, and/or anti-BMPR2 monoclonal antibody of the invention are administered simultaneously with an antibody having distinct binding specificity such as, for example, anti-CTLA-4 and/or anti-PD-1, in which case the dosage of each antibody administered falls within the ranges indicated.

Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated and typically until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition and mode of administration, without being toxic to the patient. The selected dosage level will depend upon a variety of pharmacokinetic factors including the activity of the particular compositions of the present invention employed or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated and like factors well known in the medical arts.

A “therapeutically effective dosage” of an anti-BMP2, anti-BMP4, anti-BMPR1A, anti-BMPR1B, anti-ACTR1, and/or anti-BMPR2 antibody of the invention typically results in a decrease in severity of disease symptoms, an increase in frequency and duration of disease symptom-free periods or a prevention of impairment or disability due to the disease affliction. For example, for the treatment of bone disease or cancers associated with BMP2, BMP4, BMPR1A, BMPR1B, ACTR1, and/or BMPR2 cells or tumors, a “therapeutically effective dosage” typically inhibits cell growth or tumor growth by at least about 20%, more typically by at least about 40%, even more typically by at least about 60% and still more typically by at least about 80% relative to untreated subjects. The ability of a compound to inhibit tumor growth can be evaluated in an animal model system predictive of efficacy in human tumors. Alternatively, this property of a composition can be evaluated by examining the ability of the compound to inhibit cell growth, such inhibition can be measured in vitro by assays known to the skilled practitioner. A therapeutically effective amount of a therapeutic compound can decrease tumor size or otherwise ameliorate symptoms in a subject. One of ordinary skill in the art would be able to determine such amounts based on such factors as the subject's size, the severity of the subject's symptoms and the particular composition or route of administration selected.

A composition of the present invention can be administered via one or more routes of administration using one or more of a variety of methods known in the art. As will be appreciated by the skilled artisan, the route and/or mode of administration will vary depending upon the desired results. Preferred routes of administration for antibodies of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an antibody of the invention can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches and microencapsulated delivery systems. Biodegradable, biocompatible polymers can be used, such as ethylene .vinyl acetate, polyanhydrides, polyglycolic acid, collagen, polyorthoesters and polylactic acid. Many methods for the preparation of such formulations are patented or generally known to those skilled in the art. See, e.g., Sustained and Controlled Release Drug Delivery Systems (J. R. Robinson, ed., Marcel Dekker, Inc., New York, 1978).

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems and modules are known to those skilled in the art.

In certain embodiments, the human monoclonal antibodies of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. Liposomes may comprise one or more moieties which are selectively transported into specific cells or organs, thus enhance targeted drug delivery (see, e.g., Ranade, J. Clin. Pharmacol. 29:685 (1989)). Exemplary targeting moieties include folate or biotin (see, e.g., U.S. Pat. No. 5,416,016 to Low et al.); mannosides (Umezawa et al., Biochem. Biophys. Res. Commun. 153:1038 (1988)); antibodies (Bloeman et al. FEBS Lett. 357:140 (1995); Owais et al. Antimicrob. Agents Chemother. 39:180 (1995)); surfactant protein A receptor (Briscoe et al. Am. J. Physiol. 1233:134 (1995)); Schreier et al. J. Biol. Chem. 269:9090 (1994)); see, also, Keinanen and Laukkanen FEBS Lett. 346:123 (1994); Killion and Fidler Immunomethods 4:273 (1994).

### Uses and Methods of the Invention

The antibodies, particularly the human antibodies, antibody compositions and methods of the present invention have numerous in vitro and in vivo diagnostic and therapeutic utilities involving the diagnosis and treatment of BMP2, BMP4, BMPR1A, BMPR1B, ACTR1, and/or BMPR2 mediated disorders. For example, these molecules can be administered to cells in culture, in vitro or ex vivo or to human subjects, e.g., in vivo, to treat, prevent and to diagnose a variety of disorders. As used herein, the term “subject” is intended to include human and non-human animals. The term “non-human animals” includes all vertebrates, e.g., mammals and non-mammals, such as non-human primates, sheep, dogs, cats, cows, horses, chickens, amphibians and reptiles. Preferred subjects include human patients having disorders mediated by BMP2, BMP4, BMPR1A, BMPR1B, ACTR1, and/or BMPR2 activity. The methods are particularly suitable for treating human patients having a disorder mediated by BMP2, BMP4, BMPR1A, BMPR1B, ACTR1, and/or BMPR2 expression or function. When antibodies to BMP2, BMP4, BMPR1A, BMPR1B, ACTR1, and/or BMPR2 are administered together with another agent, the two can be administered either in order or simultaneously.

Given the specific binding of the antibodies of the invention for BMP2, BMP4, BMPR1A, BMPR1B, ACTR1, and/or BMPR2, the antibodies of the invention can be used to specifically detect BMP2, BMP4, BMPR1A, BMPR1B, ACTR1, and/or BMPR2 expression by cells and tissues, moreover, can be used to purify BMP2, BMP4, BMPR1A, BMPR1B, ACTR1, and/or BMPR2 via immunoaffinity purification.

As described above, BMP2, BMP4, BMPR1A, BMPR1B, ACTR1, and/or BMPR2 are associated with a variety of diseases involving inflammation and abnormal bone formation and ossification. These diseases include Spondyloarthritides (SpA) diseases that, together, are characterized by spinal inflammation, significant pain, and functional disability. SpA diseases include, for example, ankylosing spondylitis, psoriatic spondyloarthritides, reactive spondyloarthritides, spondyloarthritides associated with inflammatory bowel disease, and undifferentiated spondyloarthritides. In particular, anti-BMP2, anti-BMP4, anti-BMPR1A, anti-BMPR1B, anti-ACTR1, and/or anti-BMPR2 antibodies of the present invention may be effective in the treatment of ankylosing spondylitis (AS), other spondyloarthropathies, and related inflammatory rheumatic diseases, which are typically characterized by inflammatory back pain, usually caused by sacroiliitis and enthesitis. Thus, the invention encompasses methods of treating the aforementioned diseases comprising administering the monoclonal antibodies disclosed herein to a subject.

The current standard of treatment for many AS patients includes TNFα blockade. Therapy with TNFα blockade has shown to be effective in reducing symptoms of the disorder, likely by reducing chronic inflammation which contributes to the disease pathology. However, there are negative consequences that can occur after prolonged use of TNFα blockers. These include, for example, increased incidence of tuberculosis, allergic reactions, and hematological disorders such as anemia. In addition, TNFα blockade is contraindicated for those with congestive heart failure.

To overcome the difficulties associated with TNFα blockade treatment, the antibodies of the invention may be used in combination with TNFα blockade for the treatment of AS. The combination of one or more anti-BMP2, anti-BMP4, anti-BMPR1A, anti-BMPR1B, anti-ACTR1, and/or anti-BMPR2 antibodies with TNFα blockade is advantageous in that the combination may result in synergy between the two therapies resulting in treatment or prevention of disease progression. Indeed, the amount or frequency of TNFα blockade can be reduced when used in combination with an antibody of the invention. This combination therapy mitigates some of the negative consequences of prolonged TNFα blockade. It has been reported (Kaplan et al, J. of Bone and Joint Surgery 2007 89:347-357) that abrogation of inflammation is ineffective at inhibiting heterotopic bone formation once the endochondral anlagen is induced. Thus, the combination of one or more anti-BMP2, anti-BMP4, anti-BMPR1A, anti-BMPR1B, anti-ACTR1, and/or anti-BMPR2 antibodies and a palliative, such as, for example, TNFα blockade, could prove to be effective in treating or preventing AS disease progression and ameliorating its symptoms.

Anti-BMP2, anti-BMP4, anti-BMPR1A, anti-BMPR1B, anti-ACTR1, and/or anti-BMPR2 antibodies are additionally used to treat other diseases and medical conditions with abnormal bone formation or ossification including fibrodysplasia ossificans progressiva (FOP) (Kan et al., Am. J. Path. 165(4):1107-15 (2004)), progressive osseous heteroplasia (POH), spinal chord injury, blunt trauma resulting in intramuscular hematoma, orthopedic surgery, psoriatic arthritis, osteoarthritis, ankylosing spondylitis, seronegative anthropathies, skeletal hyperpstosis, otosclerosis, stapes ankylosis, bone cancers, prostate cancer and exotoses, artherosclerosis, valvular heart disease, and post-operative resynostosis.

Medical conditions that involve heterotopic bone formation sometimes also include bone loss, or osteolysis, in normal bone. Thus, the present invention includes treatment of patients having heterotopic bone formation with one or more anti-BMP2, anti-BMP4, anti-BMPR1A, anti-BMPR1B, anti-ACTR1, and/or anti-BMPR2 antibodies in combination with inhibitors of bone resorption including, but not limited to, bisphosphonates, PTH inhibitors, direct and indirect inhibitors of RANKL, and inhibitors of other osteoclastic factors, such as MCSF (see WO 2005/068503, the contents of which are expressly incorporated herein by reference).

BMP2, BMP4, BMPR1A, BMPR1B, ACTR1, and/or BMPR2 are also expressed in a variety of human cancers including bone cancers, prostate cancers, lung cancers, melanomas and other hematopoietic cancers, and breast cancers. One or more anti-BMP2, anti-BMP4, anti- BMPR1A, anti-BMPR1B, anti-ACTR1, and/or anti-BMPR2 antibody may be used alone to inhibit the growth or metastasis of cancerous tumors. Alternatively, one or more anti-BMP2, anti-BMP4, anti-BMPR1A, anti-BMPR1B, anti-ACTR1, and/or anti-BMPR2 antibodies may be used in conjunction with other immunogenic agents, standard cancer treatments or other antibodies, as described herein.

Preferred cancers whose growth or metastasis may be inhibited using the antibodies of the invention include cancers typically responsive to immunotherapy. Non-limiting examples of preferred cancers for treatment include breast cancer (e.g., breast cell carcinoma), ovarian cancer (e.g., ovarian cell carcinoma), brain tumors, chronic or acute leukemias including acute myeloid leukemia, chronic myeloid leukemia, acute lymphoblastic leukemia, chronic lymphocytic leukemia, lymphomas (e.g., Hodgkin's and non-Hodgkin's lymphoma, lymphocytic lymphoma, primary CNS lymphoma, T-cell lymphoma) and nasopharangeal carcinomas. Examples of other cancers that may be treated using the methods of the invention include melanoma (e.g., metastatic malignant melanoma), prostate cancer, colon cancer, lung cancer, bone cancer, pancreatic cancer, skin cancer, cancer of the head or neck, cutaneous or intraocular malignant melanoma, uterine cancer, rectal cancer, cancer of the anal region, stomach cancer, renal cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the breast gland, sarcoma of soft tissue, cancer of the urethra, cancer of the penis, solid tumors of childhood, cancer of the bladder, cancer of the kidney or ureter, carcinoma of the breast pelvis, neoplasm of the central nervous system (CNS), tumor angiogenesis, spinal axis tumor, brain stem glioma, pituitary adenoma, Kaposi's sarcoma, epidermoid cancer, squamous cell cancer, environmentally induced cancers including those induced by asbestos, e.g., mesothelioma and combinations of said cancers.

Furthermore, given the expression of BMP2, BMP4, BMPR1A, BMPR1B, ACTR1, and/or BMPR2 on various tumor cells, the human antibodies, antibody compositions and methods of the present invention can be used to treat a subject with a tumorigenic disorder, e.g., a disorder characterized by the presence of tumor cells expressing BMP2, BMP4, BMPR1A, BMPR1B, ACTR1, and/or BMPR2 including, for example, breast cancer (e.g., breast cell carcinoma), ovarian cancer (e.g., ovarian cell carcinoma), glioblastoma, brain tumors, nasopharangeal carcinomas, non-Hodgkin's lymphoma (NHL), acute lymphocytic leukemia (ALL), chronic lymphocytic leukemia (CLL), Burkitt's lymphoma, anaplastic large-cell lymphomas (ALCL), multiple myeloma, cutaneous T-cell lymphomas, nodular small cleaved-cell lymphomas, lymphocytic lymphomas, peripheral T-cell lymphomas, Lennert's lymphomas, immunoblastic lymphomas, T-cell leukemia/lymphomas (ATLL), adult T-cell leukemia (T-ALL), entroblastic/centrocytic (eb/cc) follicular lymphomas cancers, diffuse large cell lymphomas of B lineage, angioimmunoblastic lymphadenopathy (AILD)-like T cell lymphoma, HIV associated body cavity based lymphomas, embryonal carcinomas, undifferentiated carcinomas of the rhino-pharynx (e.g., Schmincke's tumor), Castleman's disease, Kaposi's Sarcoma, multiple myeloma, Waldenstrom's macroglobulinemia and other B-cell lymphomas.

Accordingly, in one embodiment, the invention provides a method of inhibiting growth of tumor cells in a subject, comprising administering to the subject a therapeutically effective amount of an anti-BMP2, anti-BMP4, anti-BMPR1A, anti-BMPR1B, anti-ACTR1, and/or anti-BMPR2 antibody or antigen-binding portion thereof. Typically, the antibody is a human antibody. Additionally or alternatively, the antibody may be a chimeric or humanized anti-BMP2, anti-BMP4, anti-BMPR1A, anti-BMPR1B, anti-ACTR1, and/or anti-BMPR2 antibody.

In one embodiment, the antibodies (e.g., human monoclonal antibodies, multispecific and bispecific molecules and compositions) of the invention can be used to detect levels of BMP2, BMP4, BMPR1A, BMPR1B, ACTR1, and/or BMPR2 or levels of cells which contain BMP2, BMP4, BMPR1A, BMPR1B, ACTR1, and/or BMPR2 on their membrane surface, which levels can then be linked to certain disease symptoms. Alternatively, the antibodies can be used to inhibit or block BMP2, BMP4, BMPR1A, BMPR1B, ACTR1, and/or BMPR2 function which, in turn, can be linked to the prevention or amelioration of certain disease symptoms, thereby implicating BMP2, BMP4, BMPR1A, BMPR1B, ACTR1, and/or BMPR2 as a mediator of the disease. This can be achieved by contacting an experimental sample and a control sample with the anti-BMP2, anti-BMP4, anti-BMPR1A, anti-BMPR1B, anti-ACTR1, and/or anti-BMPR2 antibody under conditions that allow for the formation of a complex between the antibody and BMP2, BMP4, BMPR1A, BMPR1B, ACTR1, and/or BMPR2. Any complexes formed between the antibody and BMP2, BMP4, BMPR1A, BMPR1B, ACTR1, and/or BMPR2 are detected and compared in the experimental sample and the control.

In another embodiment, the antibodies (e.g., human antibodies, humanized antibodies, multispecific and bispecific molecules and compositions) of the invention can be initially tested for binding activity associated with therapeutic or diagnostic use in vitro. For example, compositions of the invention can be tested using the flow cytometric assays described in the Examples below.

The antibodies (e.g., human antibodies, humanized antibodies, multispecific and bispecific molecules, immunoconjugates and compositions) of the invention have additional utility in therapy and diagnosis of BMP2, BMP4, BMPR1A, BMPR1B, ACTR1, and/or BMPR2 related diseases. For—example, the human monoclonal antibodies, the multispecific or bispecific molecules and the immunoconjugates can be used to elicit in vivo or in vitro one or more of the following biological activities: to inhibit the growth of and/or kill a cell expressing BMP2, BMP4, BMPR1A, BMPR1B, ACTR1, and/or BMPR2; to mediate phagocytosis or ADCC of a cell expressing BMP2, BMP4, BMPR1A, BMPR1B, ACTR1, and/or BMPR2 in the presence of human effector cells; or to block BMP2 and/or BMP4 binding to BMPR1A, BMPR1B, ACTR1, and/or BMPR2.

Suitable routes of administering the antibody compositions (e.g., human monoclonal antibodies, humanized antibodies, multispecific and bispecific molecules and immunoconjugates) of the invention in vivo and in vitro are well known in the art and can be selected by those of ordinary skill. For example, the antibody compositions can be administered by injection (e.g., intravenous or subcutaneous). Suitable dosages of the molecules used will depend on the age and weight of the subject and the concentration and/or formulation of the antibody composition.

As previously described, human anti-BMP2, anti-BMP4, anti-BMPRI A, anti-BMPR1B, anti-ACTR1, and/or anti-BMPR2 antibodies of the invention can be co-administered with one or other more therapeutic agents, e.g., a cytotoxic agent, a radiotoxic agent or an immunosuppressive agent. The antibody can be linked to the agent (as an immunocomplex) or can be administered separate from the agent. In the latter case (separate administration), the antibody can be administered before, after or concurrently with the agent or can be co-administered with other known therapies, e.g., an anti-cancer therapy, e.g., radiation. Such therapeutic agents include, among others, anti-neoplastic agents such as doxorubicin (adriamycin), cisplatin bleomycin sulfate, carmustine, chlorambucil and cyclophosphamide hydroxyurea which, by themselves, are only effective at levels which are toxic or subtoxic to a patient. Cisplatin is intravenously administered as a 100 mg/kg dose once every four weeks and adriamycin is intravenously administered as a 60-75 mg dose once every 21 days. Co-administration of the human anti-BMP2, anti-BMP4, anti-BMPR1A, anti-BMPR1B, anti-ACTR1, and/or anti-BMPR2 antibodies or antigen binding fragments thereof, of the present invention with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibody.

Target-specific effector cells, e.g., effector cells linked to compositions (e.g., human antibodies, multispecific and bispecific molecules) of the invention can also be used as therapeutic agents. Effector cells for targeting can be human leukocytes such as macrophages, neutrophils or monocytes. Other cells include eosinophils, natural killer cells and other IgG- or IgA-receptor bearing cells. If desired, effector cells can be obtained from the subject to be treated. The target-specific effector cells can be administered as a suspension of cells in a physiologically acceptable solution. The number of cells administered can be in the order of 10⁸-10⁹ but will vary depending on the therapeutic purpose. In general, the amount will be sufficient to obtain localization at the target cell, e.g., a tumor cell expressing BMP2, BMP4, BMPR1A, BMPR1B, ACTR1, and/or BMPR2 and to effect cell killing by, e.g., phagocytosis. Routes of administration can also vary.

Therapies with target-specific effector cells can be performed in conjunction with other techniques for removal of targeted cells. For example, anti-tumor therapy using the compositions (e.g., human antibodies, multispecific and bispecific molecules) of the invention and/or effector cells armed with these compositions can be used in conjunction with chemotherapy. Additionally, combination immunotherapy may be used to direct two distinct cytotoxic effector populations toward tumor cell rejection. For example, anti-BMP2, anti-BMP4, anti-BMPR1A, anti-BMPR1B, anti-ACTR1, and/or anti-BMPR2 antibodies linked to anti-Fe-gamma RI or anti-CD3 may be used in conjunction with IgG- or IgA-receptor specific binding agents.

Bispecific and multispecific molecules of the invention can also be used to modulate FcγR or FcγR levels on effector cells, such as by capping and elimination of receptors on the cell surface. Mixtures of anti-Fc receptors can also be used for this purpose.

The compositions (e.g., human, humanized, or chimeric antibodies, multispecific and bispecific molecules and immunoconjugates) of the invention which have complement binding sites, such as portions from IgG1, IgG2, IgG3, or IgM, which bind complement, can also be used in the presence of complement. In one embodiment, ex vivo treatment of a population of cells comprising target cells with a binding agent of the invention and appropriate effector cells can be supplemented by the addition of complement or serum containing complement. Phagocytosis of target cells coated with a binding agent of the invention can be improved by binding of complement proteins. In another embodiment target cells coated with the compositions (e.g., human antibodies, multispecific and bispecific molecules) of the invention can also be lysed by complement. In yet another embodiment, the compositions of the invention do not activate complement.

The compositions (e.g., human, humanized, or chimeric antibodies, multispecific and bispecific molecules and immunoconjugates) of the invention can also be administered together with complement. Accordingly, within the scope of the invention are compositions comprising human antibodies, humanized antibodies, multispecific or bispecific molecules and serum or complement. These compositions are advantageous in that the complement is located in close proximity to the human antibodies, multispecific or bispecific molecules. Alternatively, the human antibodies, multispecific or bispecific molecules of the invention and the complement or serum can be administered separately.

Also within the scope of the present invention are kits comprising the antibody compositions of the invention (e.g., human antibodies, bispecific or multispecific molecules or immunoconjugates) and instructions for use. The kit can further contain one ore more additional reagents, such as an immunosuppressive reagent, a cytotoxic agent or a radiotoxic agent or one or more additional human antibodies of the invention (e.g., a human antibody having a complementary activity which binds to an epitope in the BMP2, BMP4, BMPR1A, BMPR1B, ACTR1, and/or BMPR2 antigen distinct from the first human antibody).

Accordingly, patients treated with antibody compositions of the invention can be additionally administered (prior to, simultaneously with or following administration of a human antibody of the invention) with another therapeutic agent, such as a cytotoxic or radiotoxic agent, which enhances or augments the therapeutic effect of the human antibodies.

In other embodiments, the subject can be additionally treated with an agent that modulates, e.g., enhances or inhibits, the expression or activity of Fcγ or Fcγ receptors by, for example, treating the subject with a cytokine. Preferred cytokines for administration during treatment with the multispecific molecule include of granulocyte colony-stimulating factor (G-CSF), granulocyte-macrophage colony-stimulating factor (GM-CSF), interferon-γ (IFN-γ) and tumor necrosis factor (TNF).

The compositions (e.g., human antibodies, humanized antibodies, multispecific and bispecific molecules) of the invention can also be used to target cells expressing FcγR or one or more of BMP2, BMP4, BMPR1A, BMPR1B, ACTR1, and/or BMPR2, for example for labeling such cells. For such use, the binding agent can be linked to a molecule that can be detected. Thus, the invention provides methods for localizing ex vivo or in vitro cells expressing Fc receptors, such as FcγR and/or one or more of BMP2, BMP4, BMPR1A, BMPR1B, ACTR1, and/or BMPR2. The detectable label can be, e.g., a radioisotope, a fluorescent compound, an enzyme or an enzyme co-factor.

In a particular embodiment, the invention provides methods for detecting the presence of BMP2, BMP4, BMPR1A, BMPR1B, ACTR1, and/or BMPR2 antigen in a sample or measuring the amount of BMP2, BMP4, BMPR1A, BMPR1B, ACTR1, and/or BMPR2 antigen, comprising contacting the sample and a control sample, with a human monoclonal antibody or an antigen binding portion thereof, which specifically binds to BMP2, BMP4, BMPR1A, BMPR1B, ACTR1, and/or BMPR2, under conditions that allow for formation of a complex between the antibody or portion thereof and BMP2, BMP4, BMPR1A, BMPR1B, ACTR1, and/or BMPR2. The formation of a complex is then detected, wherein a difference complex formation between the sample compared to the control sample is indicative the presence of BMP2, BMP4, BMPR1A, BMPR1B, ACTR1, and/or BMPR2 antigen in the sample.

In yet another embodiment, immunoconjugates of the invention can be used to target compounds (e.g., therapeutic agents, labels, cytotoxins, radiotoxoins immunosuppressants, etc.) to cells which have BMPR1A, BMPR1B, ACTR1, and/or BMPR2 cell surface receptors by linking such compounds to the antibody. For example, a BMPR1A, BMPR1B, ACTR1, and/or BMPR2 antibody can be conjugated to UPT, as described in U.S. Pat. No. 6,989,452, U.S. patent application Ser. Nos. 10/160,972, 10/161,234, 11/134,826, 11/134,685, and U.S. Provisional Patent Application No. 60/720,499, and/or any of the toxin compounds described in U.S. Pat. Nos. 6,281,354 and 6,548,530, U.S. patent publication Nos. 20030050331, 20030064984, 20030073852 and 20040087497 or published in WO 03/022806, which are hereby incorporated by reference in their entireties. Thus, the invention also provides methods for localizing ex vivo or in vivo cells expressing BMPR1A, BMPR1B, ACTR1, and/or BMPR2 (e.g., with a detectable label, such as a radioisotope, a fluorescent compound, an enzyme or an enzyme co-factor). Alternatively, the immunoconjugates can be used to kill cells which have BMPR1A, BMPR1B, ACTR1, and/or BMPR2 cell surface receptors by targeting cytotoxins or radiotoxins to BMPR1A, BMPR1B, ACTR1, and/or BMPR2.

The present invention is further illustrated by the following examples which should not be construed as further limiting. The contents of all figures and all references, patents and published patent applications cited throughout this application are expressly incorporated herein by reference.

### EXAMPLES

### Example 1

### Generation of Human Monoclonal Antibodies Against BMP2, BMP4, BMPR1A, BMPR1B, ACTR1, and BMPR2

This Example discloses methodology for the generation of human monoclonal antibodies that specifically bind to human BMP2, BMP4, BMPR1A, BMPR1B, ACTR1, and BMPR2.

### Antigen

Mice are immunized with recombinant human BMP2, BMP4, BMPR1A, BMPR1B, ACTR1, and/or BMPR2. In particular, mice were immunized with commercially available recombinant human BMP2 or BMP4. Human recombinant BMP-2 was obtained from R&D Systems, Inc. (Catalog No. 355-BM/CF, Lot.-MSA10605H) or Medtronic, Inc (Lot.- M115006AAJ). Human recombinant BMP4 was obtained from R&D Systems, Inc (Catalog No. 31-BP/CF, Lots BEM186051 and BEM316071 and MSA10605H). The lyophilized antigens were reconstituted according to the manufacturer instructions and stored at −20° C.

### Transgenic HuMAb Mouse® and KM Mouse®

Fully human monoclonal antibodies to BMP2, BMP4, BMPR1A, BMPR1B, ACTR1, and BMPR2 may be prepared using the HCo7, HCo12 and HCo17 strains of HuMab transgenic mice or a KM transgenic mouse, which expresses human antibody genes. In these mouse strains, the endogenous mouse kappa light chain gene has been homozygously disrupted as described in Chen et al. (1993) EMBO J. 12:811-820 and the endogenous mouse heavy chain gene has been homozygously disrupted as described in Example 1 of PCT Publication WO 01/09187. Furthermore, this mouse strain carries a human kappa light chain transgene, KCo5, as described in Fishwild et al. Nature Biotechnology 14:845-851 (1996) and a human heavy chain transgene, HCo7, HCo12 or HCo17 as described in Example 2 of PCT Publication WO 01/09187.

Fully human monoclonal antibodies to BMP-2 and BMP-4 were prepared using HCo20:02{M/K} (Balb) F1 and HCo27:04{M/K} strains of the transgenic HuMAb Mouse® and the KM strain of transgenic transchromosomic mice, each of which express human antibody genes. The HCo20:02{M/K} (Balb) F1 and HCo27:04{MIK} mice were constructed as described in WO 2005/058815, which is incorporated herein by reference in its entirety. The KM strain was constructed as described in WO 02/43478, which is incorporated herein by reference in its entirety.

### HuMAb Mouse® and KM Mouse® Immunizations

To generate fully human monoclonal antibodies to human BMP2 and BMP4, mice of the HuMAb Mouse® and KM Mouse® were immunized with either human recombinant BMP2 or BMP4. General immunization schemes for the HuMAb Mouse® are described in Lonberg, N. et al (1994) Nature 368(6474): 856-859; Fishwild, D. et al. (1996) Nature Biotechnology 14: 845-851 and PCT Publication WO 98/24884. The mice were 6-16 weeks of age upon the first infusion of antigen. A purified preparation (10-15 μg) of recombinant BMP2 or BMP4 was used to immunize each HuMab mouse® and KM mouse®.

Transgenic mice were immunized with antigen emulsified in Ribi adjuvant either intraperitonealy and subcutaneously or via footpad at one week intervals for up to 12 immunizations. Mice selected for B cell fusions were further immunized intravenously and intraperitonealy with antigen 3 days and again one day prior the splenectomy. The immune response was monitored by retroorbital bleeds. The plasma was screened by ELISA (as described below) and mice with sufficient titers of anti-BMP2 and BMP4 human immunoglobulin were used for fusions. Mice were boosted intravenously with antigen 3 and 1 days before sacrifice and removal of the spleen. Four fusions were performed and a total of 33 mice were immunized.

### Selection of HuMab Mouse® or KM Mouse® Producing Anti-BMP2, Anti-BMP4,

### Anti-BMPR1A, Anti-BMPR1B, Anti-ACTR1, and Anti-BMPR2 Antibodies

To select a HuMab Mouse™ or a KM Mouse™ producing antibodies that bind BMP2, BMP4, BMPR1A, BMPR1B, ACTR1, and/or BMPR2, sera from immunized mice are screened by ELISA using purified antigen adsorbed to microtiter plates as described by Fishwild et al. (1996), supra.

In particular, microtiter plates were coated with purified recombinant BMP2 or BMP4 at 1-2 μg/ml in PBS, 50 μl/wells, incubated at ambient temperature overnight, washed four times with PBS/Tween (0.05%) and then blocked with 200 μl/well of PBS/Tween (0.05%) supplemented with 0.5% bovine serum albumin (BSA). Dilutions of plasma from BMP2 or BMP4 immunized mice were added to each well and incubated for 1-2 hours at ambient temperature. The plates were washed with PBS/Tween (0.05%) and then incubated with a goat-anti-human IgG Fc specific polyclonal antibody conjugated with horseradish peroxidase (HRP) for 1 hour at room temperature. After washing, the plates were developed with ABTS substrate (Moss, Inc. Cat. No. ABTS-1000) and analyzed by spectrophotometer at OD 415-495.

Mice that develop the highest titers of antigen-specific antibodies may be used for fusions. Fusions are performed as described below and hybridoma supernatants are tested for anti-BMP2, anti-BMP4, anti-BMPR1A, anti-BMPR1B, anti-ACTR1, and/or anti-BMPR2 activity by ELISA. Antibodies that bind to the antigen adsorbed to a microtitre plate may be, for example, expressed as a fusion protein in CHO cells, but not the parental CHO cells. The antibodies are identified by flow cytometry for binding to a cell line expressing recombinant human antigen, but not to a control cell line that does not express the respective antigen. Binding of anti-BMP2, anti-BMP4, anti-BMPR1A, anti-BMPR1B, anti-ACTR1, and anti-BMPR2 antibodies may be assessed, for example, by incubating antigen-expressing CHO cells with the antibody or interest at a concentration of 20 μg/ml. The cells are washed and binding is detected with a label such as FITC conjugated to an anti-human IgG Ab. Flow cytometric analyses are performed using a FACScan flow cytometry (Becton Dickinson, San Jose, Calif.).

### Generation of Hybridomas Producing Human Monoclonal Antibodies to BMP2, BMP4, BMPR1A, BMPR1B, ACTR1, and BMPR2

Hybridomas producing human monoclonal antibodies to BMP2, BMP4, BMPR1A, BMPR1B, ACTR1, and BMPR2 are produced, for example, using the protocol described below. In particular, mouse splenocytes, isolated from a HuMab mouse® or a KM mouse® immunized with BMP2, were fused using electric field based electrofusion using a Cyto Pulse large chamber cell fusion electroporator (Cyto Pulse Sciences, Inc., Glen Burnie, Md.). The resulting hybridomas were then screened for the production of antigen-specific antibodies using an antibody capture Elisa assay. Single cell suspensions of splenic lymphocytes from immunized mice were fused with Ag8.653 nonsecreting mouse myeloma cells (ATCC, CRL 1580) using electric field based electrofusion using a Cyto Pulse large chamber cull fusion electroporator (Cyto Pulse Sciences, Inc., Glen Burnie, Md.). Cells were plated at approximately 1×10⁴ cells/well in flat bottom microtiter plates, followed by a two week incubation in selective medium containing 10% fetal bovine serum 388D1 (ATCC, CRL TIB-63) conditioned medium, 3-5% Hybridoma cloning factor (Bioveris, Inc.) in DMEM (Mediatech, CRL 10013, with high glucose, L-glutamine and sodium pyruvate) supplemented with 10 mM HEPES, 0.055 mM 2-mercaptoethanol, and 1× HAT (Sigma, CRL P-7185). After 1-2 weeks, cells were cultured in medium in which the HAT was replaced with HT. Individual wells were then screened by ELISA (described above) for human anti-BMP2 or BMP4 monoclonal IgG antibodies. Once extensive hybridoma growth occurred (10-14 days), medium was monitored for antibody production usually after 10-14 days. The antibody secreting hybridomas were further propagated in larger culture vessels and screened again for production of antigen specific antibodies. Selected colonies were cryopreserved and cloned once or twice by limiting dilution. The stable subclones were then cryopreserved and propagated in vitro to generate amounts of antibody sufficient for further characterization.

From the BMP2-immunized mice, a total of 495 hybridoma colonies that produce human anti-BMP2/4 antibodies were produced. Thirty five colonies were selected for cloning and subsequent propagation for further analysis. Among the thirty five colonies were the 6H4, 11F2, 12E3, 1F6, 10F6, 10H6, 16b7, 7D6, 8B3, 33F7, and 15F3 hybridomal cell lines.

### Example 2

### Structural Characterization of Human Monoclonal Antibodies

This Example discloses the structural characteristics of human monoclonal antibodies that specifically bind to BMP2 and BMP4. In particular, the structures of the anti-BMP2/4 monoclonal antibodies 6H4, 11F2, 12E3, 1F6, 10F6, 10H6, 16b7, 7D6, 8B3, 33F7, and 15F3 are disclosed in this example.

The cDNA sequences encoding the heavy and light chain variable regions of monoclonal antibodies derived by the methodology of Example 1 are obtained from the anti-BMP2, anti-BMP4, anti-BMPR1A, anti-BMPR1B, anti-ACTR1, and/or anti-BMPR2 hybridomas, respectively, using standard PCR techniques and are sequenced using standard DNA sequencing techniques.

The cDNA sequences encoding the heavy and light chain variable regions of the 6H4, 11 F2 and 12E3 monoclonal antibodies were obtained from the 6H4, I1F2 and 12E3 hybridomas, respectively, using standard PCR techniques and were sequenced using standard DNA sequencing techniques.

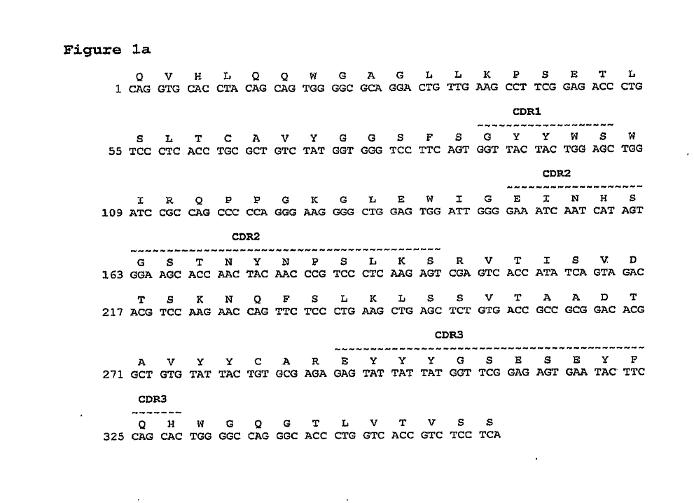

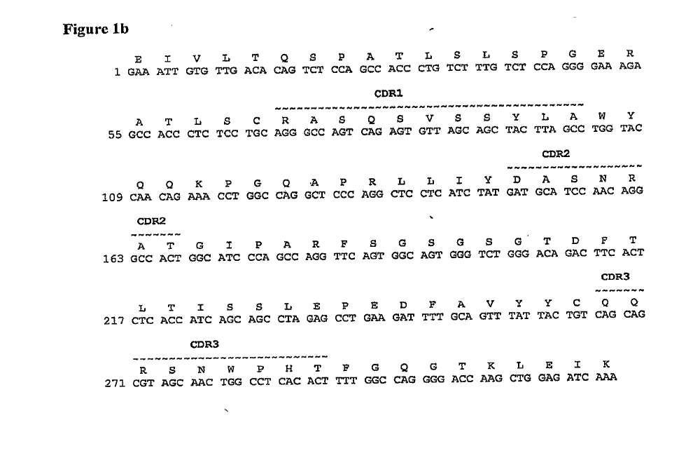

The nucleotide and amino acid sequences of the heavy chain variable region of 6H4 are shown in FIG. 1A and in SEQ ID NO:31 and 37, respectively. The nucleotide and amino acid sequences of the light chain variable region of 6H4 are shown in FIG. 1B and in SEQ ID NO:34 and 40, respectively.

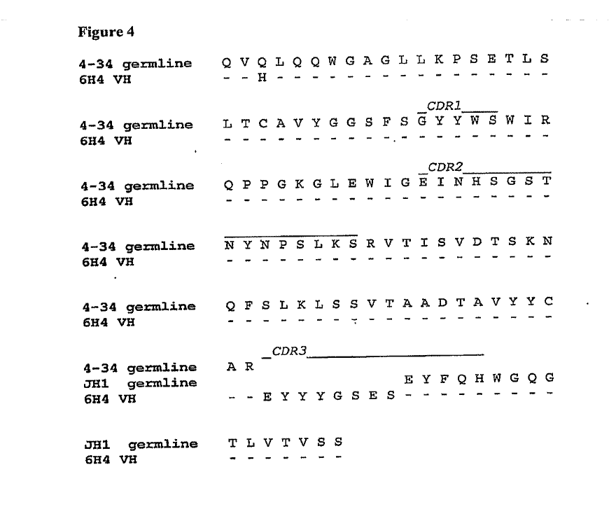

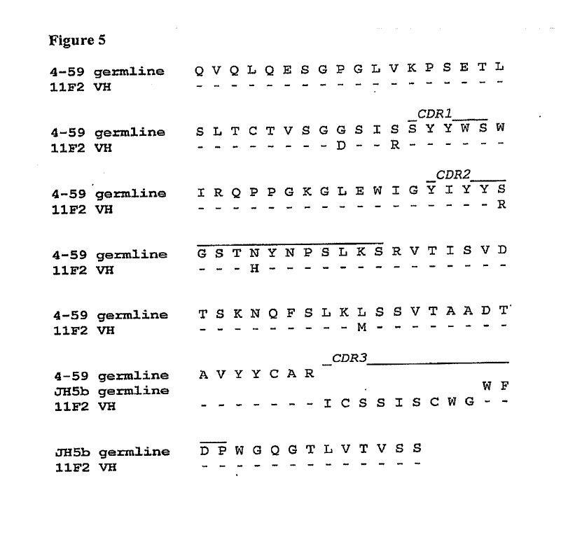

Comparison of the 6H4 heavy chain immunoglobulin sequence to the known human germline immunoglobulin heavy chain sequences demonstrated that the 6H4 heavy chain utilizes a V_{H} segment from human germline V_{H} 4-34 (SEQ ID NO:51), a D segment from the human germline 3-10 (SEQ ID NO:52), and a J_{H} segment from human germline JH1 (SEQ ID NO:53). The alignment of the 6H4 V_{H} sequence to the germline V_{H} 4-34 sequence is shown in FIG. 4. Further analysis of the 6H4 V_{H} sequence using the Kabat system of CDR region determination led to the delineation of the heavy chain CDR1, CDR2 and CD3 regions as shown in FIGS. 1A and 4, and in SEQ ID NOs:13, 16 and 19, respectively.

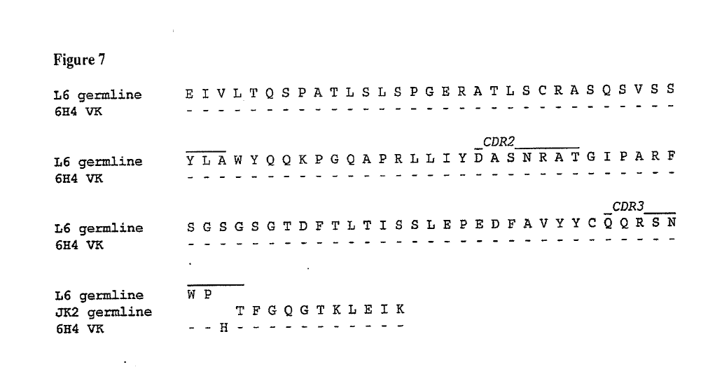

Comparison of the 6H4 light chain immunoglobulin sequence to the known human germline immunoglobulin light chain sequences demonstrated that the 6H4 light chain utilizes a V_{L} segment from human germline V_{K} L6 (SEQ ID NO:54) and a JK segment from human germline JK2 (SEQ ID NO:55). The alignment of the 6H4 V_{K} sequence to the germline V_{K} L6 sequence is shown in FIG. 7. Further analysis of the 6H4 V_{L} sequence using the Kabat system of CDR region determination led to the delineation of the light chain CDR1, CDR2 and CD3 regions as shown in FIGS. 1B and 7, and in SEQ ID NOs:22, 25, and 28, respectively.

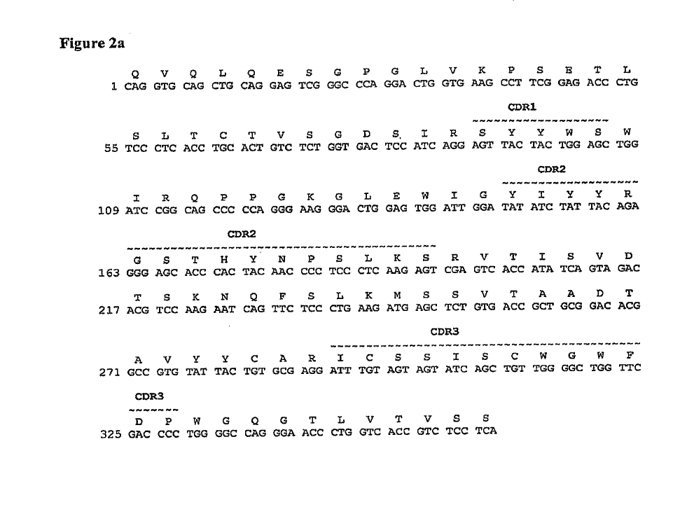

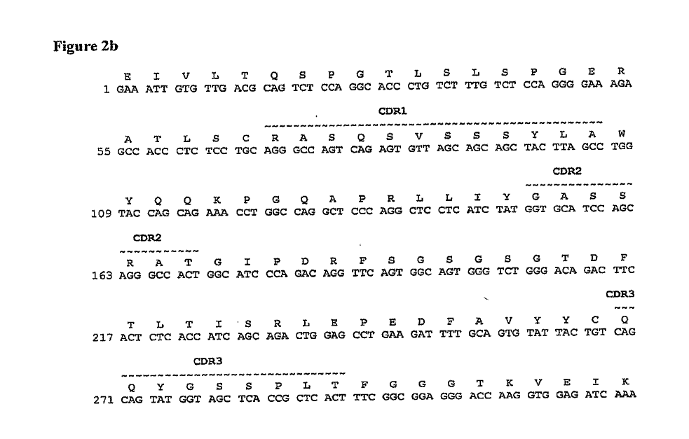

The nucleotide and amino acid sequences of the heavy chain variable region of 11F2 are shown in FIG. 2A and in SEQ ID NO:32 and 38, respectively. The nucleotide and amino acid sequences of the light chain variable region of 11F2 are shown in FIG. 2B and in SEQ ID NO:35 and 41, respectively.

Comparison of the 11F2 heavy chain immunoglobulin sequence to the known human germline immunoglobulin heavy chain sequences demonstrated that the 6H4 heavy chain utilizes a V_{H} segment from human germline V_{H} 4-59 (SEQ ID NO:43), a D segment from the human germline 2-2 (SEQ ID NO:45), and a J_{H} segment from human germline JH5b (SEQ ID NO:46). The alignment of the 11F2 V_{H} sequence to the germline V_{H} 4-59 sequence is shown in FIG. 5. Further analysis of the 11F2 V_{H} sequence using the Kabat system of CDR region determination led to the delineation of the heavy chain CDR1, CDR2 and CD3 regions as shown in FIGS. 2A and 5, and in SEQ ID NOs:14, 17 and 20, respectively.

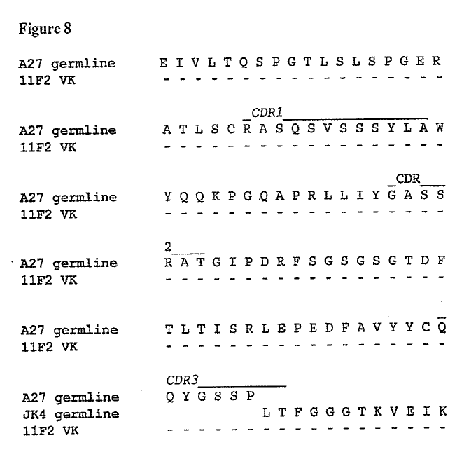

Comparison of the 11 F2 light chain immunoglobulin sequence to the known human germline immunoglobulin light chain sequences demonstrated that the 11F2 light chain utilizes a V_{L} segment from human germline V_{K} A27 (SEQ ID NO:48) and a JK segment from human germline JK4 (SEQ ID NO:50). The alignment of the 11F2 V_{K} sequence to the germline V_{K} A27 sequence is shown in FIG. 8. Further analysis of the 11F2 V_{L} sequence using the Kabat system of CDR region determination led to the delineation of the light chain CDR1, CDR2 and CD3 regions as shown in FIGS. 2B and 8, and in SEQ ID NOs:23, 26, and 29, respectively.

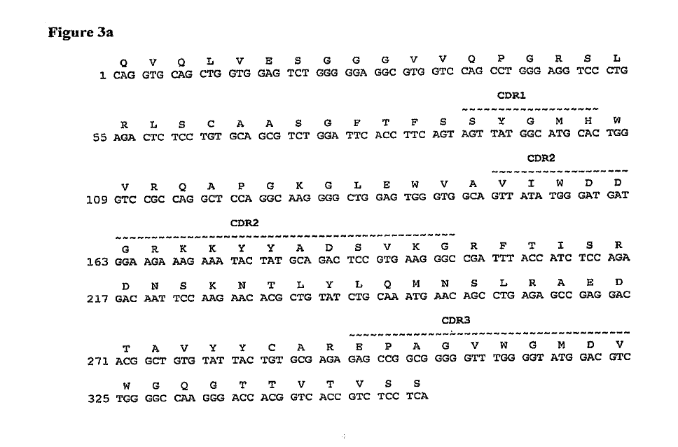

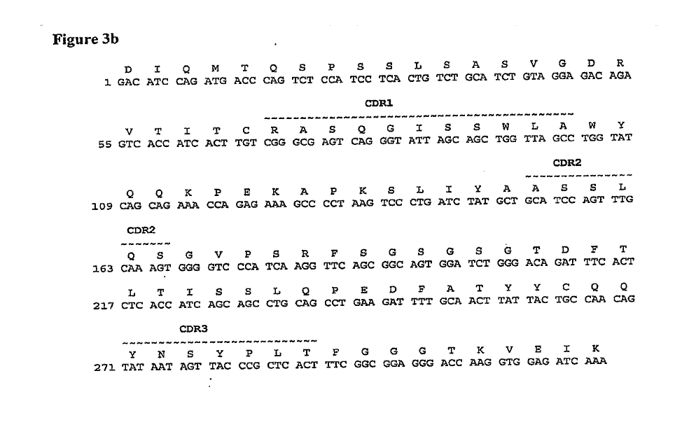

The nucleotide and amino acid sequences of the heavy chain variable region of 12E3 are shown in FIG. 3A and in SEQ ID NO:33 and 39, respectively. The nucleotide and amino acid sequences of the light chain variable region of 12E3 are shown in FIG. 3B and in SEQ ID NO:36 and 42, respectively.

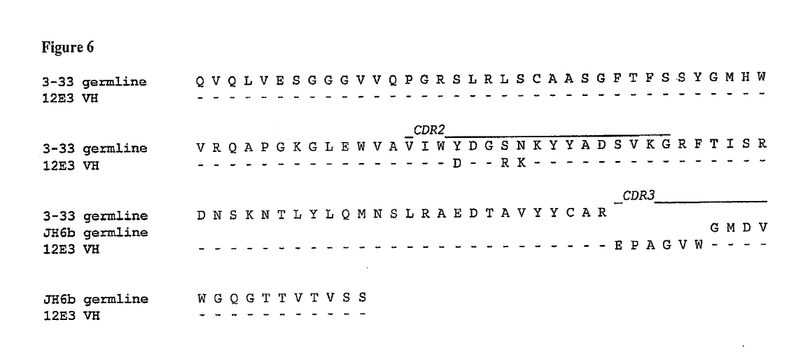

Comparison of the 12E3 heavy chain immunoglobulin sequence to the known human germline immunoglobulin heavy chain sequences demonstrated that the 12E3 heavy chain utilizes a V_{H} segment from human germline V_{H} 3-33 (SEQ ID NO:44) and a J_{H} segment from human germline JH6b (SEQ ID NO:47). The alignment of the 12E3 V_{H} sequence to the germline V_{H} 4-33 sequence is shown in FIG. 6. Further analysis of the 12E3 V_{H} sequence using the Kabat system of CDR region determination led to the delineation of the heavy chain CDR1, CDR2 and CD3 regions as shown in FIGS. 3A and 6, and in SEQ ID NOs:15, 18 and 21, respectively.

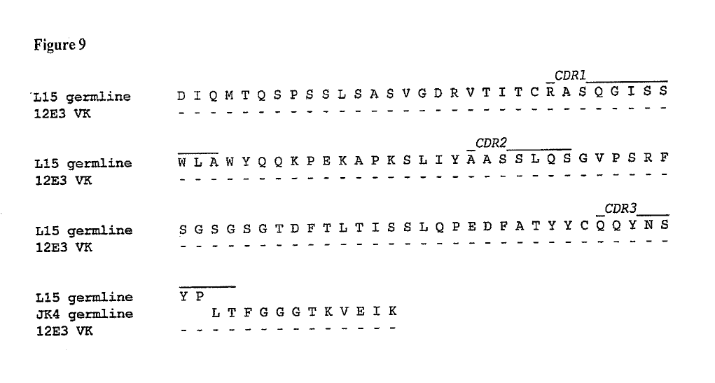

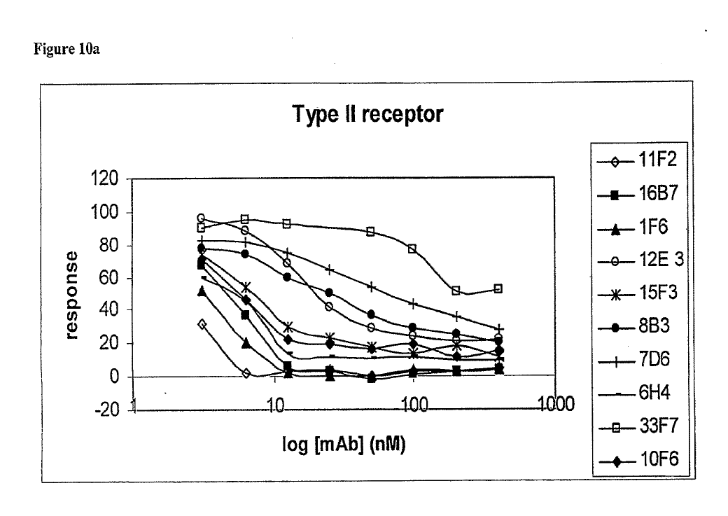

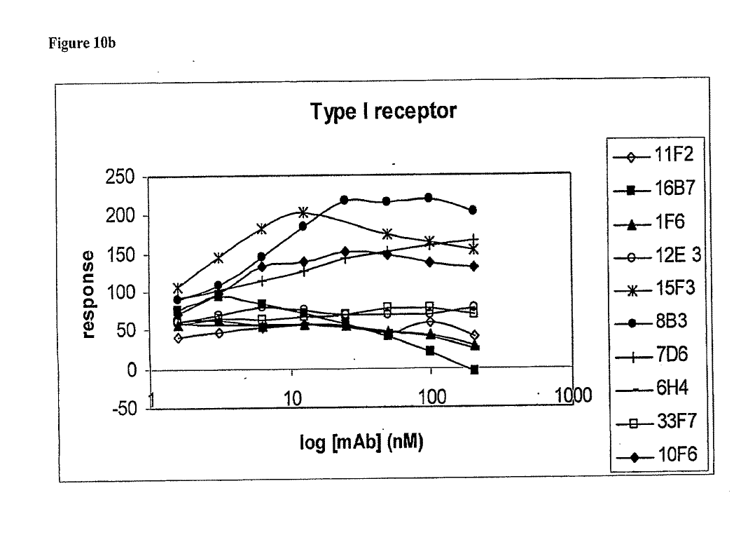

Comparison of the 12E3 light chain immunoglobulin sequence to the known human germline immunoglobulin light chain sequences demonstrated that the 12E3 light chain utilizes a V_{L} segment from human germline V_{K} L15 (SEQ ID NO:49) and a J_{K} segment from human germline JK4 (SEQ ID NO:50). The alignment of the 12E3 V_{K} sequence to the germline V_{K} L15 sequence is shown in FIG. 9. Further analysis of the 12E3 V_{L} sequence using the Kabat system of CDR region determination led to the delineation of the light chain CDR1, CDR2 and CD3 regions as shown in FIGS. 3B and 9, and in SEQ ID NOs:24, 27, and 30, respectively.

The cDNA sequences encoding the heavy and light chain variable regions of the 10F6, 10H6 and 16b7 monoclonal antibodies were obtained froth the 10F6, 10H6 and 16b7 hybridomas, respectively, using standard PCR techniques and were sequenced using standard DNA sequencing techniques. The heavy chain of the 10F6 and 10H6 monoclonal antibodies utilize human germline V_{H} 3-33 (SEQ ID NO:44), D_{H} 6-13, and J_{H} JH4b genes (SEQ ID NO:88). The light chain of the 10F6 and 10H6 monoclonal antibodies utilize human germline V_{K} L15 and J_{K} JK4 genes. The heavy chain of the 16B7 monoclonal antibody utilizes human germline V_{H} 3-33, D_{H} 6-13, and J_{H} JH2 (SEQ ID NO:89) genes. The light chain of the 16B7 monoclonal antibody utilizes human germline V_{K} L15 and J_{K} JK4 genes.

The cDNA sequence encoding the heavy and light chain variable regions of the 1F6 monoclonal antibody was obtained from the 1F6 hybridoma using standard PCR techniques and was sequenced using standard DNA sequencing techniques. The heavy chain of the 1F6 monoclonal antibody utilizes human germline V_{H} 4-59, D_{H} 2-2, and J_{H} JH5b genes. The light chain of the 1F6 monoclonal antibody utilizes human germline V_{K} A27 and J_{K} JK4 genes.

The cDNA sequences encoding the heavy and light chain variable regions of the 7D6, 8B3, 33F7, and 15F3 monoclonal antibodies were obtained from the 7D6, 8B3, 33F7, and 15F3 hybridomas, respectively, using standard PCR techniques and were sequenced using standard DNA sequencing techniques. The heavy chains of these monoclonal antibodies utilize human germline V_{H} 1-69 (SEQ ID NO:91) and J_{H} JH3b genes (SEQ ID NO:90). The light chains of these monoclonal antibodies utilize human germline V_{K} A27 and J_{K} JK2 genes.

### Example 3

### Characterization of Binding Specificity of Anti-BMP2, Anti-BMP4, Anti-BMPR1A, Anti-BMPR1B, Anti-ACTR1, and Anti-BMPR2 Monoclonal Antibodies

This Example discloses methodologies for comparing anti-BMP2, anti-BMP4, anti-BMPR1A, anti-BMPR1B, anti-ACTR1, and/or anti-BMPR2 antibodies on binding to immunopurified antigen by ELISA and western blot assays or binding to BMP2/4 in tissues using immunohistochemistry to examine the specificity of antigen binding.

Recombinant His-tagged and myc-tagged antigens are coated on a plate overnight, then tested for binding against the human monoclonal antibodies generated by the methodology disclosed in Example 1. Standard ELISA procedures are performed. The anti-BMP2, anti-BMP4, anti-BMPR1A, anti-BMPR1B, anti-ACTR1, and/or anti-BMPR2 human monoclonal antibodies are added at a concentration of 1 μg/ml and titrated down at 1:2 serial dilutions. Goat-anti-human IgG (Fc or kappa chain-specific) polyclonal antibody conjugated with horseradish peroxidase (HRP) is used as secondary antibody.

Recombinant B7H4-Ig is purified from supernatants of 293T cells transfected with a B7H4-Ig construct by chromatography using protein A. An ELISA plate is coated with the human antibodies, followed by addition of purified protein and then detection with the rabbit anti-B7H4 antisera. Recombinant Penta-B7H4 protein with a C-9 tag is purified from supernatants of 293T cells transfected with a Penta-B7H4-C9 construct by chromatography using a 2A7 affinity column. An ELISA plate is coated with anti-mouse Fc, followed by monoclonal anti-C9 (0.6 ug/ml), then titrated Penta-B7H4 as indicated, then the human monoclonal antibodies at 1 μg/ml. Coated anti-mouse Fc followed by M-anti-C9 (0.6 ug/ml), then titrated Penta-BMP2, Penta-BMP4, Penta-BMPR1A, Penta-BMPR1B, Penta-ACTR1, and/or Penta-BMPR2, then human monoclonal antibodies@1 μg/ml.

Anti-BMP2/4 antibodies were characterized for binding to BMP2 under reducing and non-reducing conditions by western blot assays. 0.5 μg of recombinant human BMP2 protein (Medtronic) was diluted directly into sample buffer (Cell Signaling, Cat #SB7722) with or without a reducing agent. Samples were heated to 100° for 3 minutes to denature the protein followed by electrophoresis and western blotting. The membrane-bound proteins were probed with 0.5 μg/ml of the test antibodies followed by detection with alkaline phosphatase conjugated Fab2 Goat anti-human IgG (Jackson ImmunoReseach Labs, cat #109-056-09) and stained with BCIP/NBT (Pierce, cat #34042). The results show that all the monoclonal antibodies tested recognize a non-reduced band at approximately 36 kDa that corresponds to the BMP2 homodimer. In addition, some of the monoclonal antibodies (e.g. 8B3) recognized BMP2 under reducing conditions. Two bands of approximately 17-18 kDa that correspond to BMP monomers were revealed.

For immunohistochemistry, 2,000 μm mouse tissue cores are used (IMGENEX Histo-Array; Imgenex Corp., San Diego, Calif.). After drying for 30 minutes, sections are fixed with acetone (at room temperature for 10 minutes) and air-dried for 5 minutes. Slides are rinsed in PBS and then pre-incubated with 10% normal goat serum in PBS for 20 min and subsequently incubated with 10 μg/ml fitcylated antibody in PBS with 10% normal goat serum for 30 min at room temperature. Next, slides are washed three times with PBS and incubated for 30 min with mouse anti-FITC (10 μg/ml DAKO) at room temperature. Slides are washed again with PBS and incubated with Goat anti-mouse HRP conjugate (DAKO) for 30 minutes at room temperature. Slides are washed again 3× with PBS. Diaminobenzidine (Sigma) is used as substrate, resulting in brown staining. After washing with distilled water, slides are counter-stained with hematoxyllin for 1 min. Subsequently, slides are washed for 10 seconds in running distilled water and mounted in glycergel (DAKO).

The epitopes recognized by a subset of the anti-BMP2/4 monoclonal antibodies was determined using receptor peptides conjugated to biotin and captured by streptavidin chip (SA chip, BIAcore) and analyzed by BIAcore. The antibodies were flowed across the chips at 40 ug/ml. The 8B3 and 7D6 antibodies bound to a BMP2 epitope (ISMLYLDENEKVVLK) (SEQ ID NO:92) that binds a BMP2 type 2 receptor. The 12E3, 11F2 and 16B7 antibodies bound a BMP2 epitope (QAKHKQRKRLKSSCKRH) (SEQ ID NO:93) that binds heparin. Additionally, the anti-BMP2/4 human monoclonal antibody 33F7 (SEQ ID NOS: 63 and 71) blocked the interaction between BMP2/4 and heparin. This blocks the function of BMP2/4.

### Example 4

### Characterization of Anti-BMP2, Anti-BMP4, Anti-BMPR1A, Anti-BMPR1B, Anti-ACTR1, and/or Anti-BMPR2 Antibody Binding to the Respective Antigen Expressed on the Surface of a Chondrocyte Cell Line

This Example discloses flow cytometry methodology for testing of anti-BMP2, anti-BMP4, anti-BMPR1A, anti-BMPR1B, anti-ACTR1, and/or anti-BMPR2 antibodies for binding to CHO-antigen transfectants and chondrocyte cells expressing BMP2, BMP4, BMPR1A, BMPR1B, ACTR1, and/or BMPR2 on their cell surface.

A CHO cell line transfected with BMP2, BMP4, BMPR1A, BMPR1B, ACTR1, and/or BMPR2 as well as the chondrocyte cell line ATDC5 (RIKEN Biosource, RCB0565) or the fibroblastic cell line MC3T3 (ATCC Accession Nos. CRL-2595, CRL-2596, CRL-2594 and CRL-2593) are tested for antibody binding. The cells are washed and binding is detected with a FITC-labeled anti-human IgG Ab. Flow cytometric analyses are performed using a FACScan flow cytometry (Becton Dickinson, San Jose, Calif.).

### Example 5

### Analysis of Binding Affinity of Anti-BMP2, Anti-BMP4, Anti-BMPR1A, Anti-BMPR1B, Anti-ACTR1, and/or Anti-BMPR2 Monoclonal Antibodies

This Example discloses methodologies for testing of monoclonal antibodies for specific binding affinity to a BMP2, BMP4, BMPR1A, BMPR1B, ACTR1, and/or BMPR2.

In one methodology, HEK cells are transfected with full length BMP2, BMP4, BMPR1A, BMPR1B, ACTR1, and/or BMPR2 using standard techniques and grown in RPMI media containing 10% fetal bovine serum (FBS). Cells are trypsinized and washed once in Tris based binding buffer (24 mM Tris pH 7.2, 137 mM NaCl, 2.7 mM KCl, 2 mM Glucose, 1 mM CaCl₂, 1 mM MgCl₂, 0.1% BSA) and adjusted to 2×10⁶ cells/ml in binding buffer. Millipore plates (MAFB NOB) are coated with 1% nonfat dry milk in water and stored at 4° C. overnight. The plates are washed three times with 0.2 ml of binding buffer. Fifty microliters of buffer alone is added to the maximum binding wells (total binding). Twenty-five microliters of buffer alone is added to the control wells (non-specific binding). Varying concentration of ¹²⁵I-antibody is added to all wells in a volume of 25 μl. Varying concentrations of unlabeled antibody at 100 fold excess is added in a volume of 25 μl to control wells and 25 μl of BMP2, BMP4, BMPR1A, BMPR1B, ACTR1, and/or BMPR2 transfected CHO cells (2×10⁶ cells/ml) in binding buffer is added to all wells. The plates are incubated for 2 hours at 200 RPM on a shaker at 4° C. Following incubation, the Millipore plates are washed three times with 0.2 ml of cold wash buffer (24 mM Tris pH 7.2, 500 mM NaCl; 2.7 mM KCl, 2 mM Glucose, 1 mM CaCl₂, 1 mM MgCl₂, 0.1% BSA.). The filters are removed and counted in a gamma counter. Evaluation of equilibrium binding is performed using single site binding parameters with the Prism software (San Diego, Calif.). Data are analyzed by non-linear regression using a sigmoidal dose response (PRIZM™) and result in calculation of an EC50, which is used to rank the antibodies for EC50 and 95% CI.

In another methodology, anti-BMP2/4 monoclonal antibodies were characterized for affinity and binding kinetics by Biacore analysis (Biacore AB, Uppsala, Sweden). The anti-BMP-2/4 antibodies were captured on a chip with an anti human Fc antibody covalently linked to a CM5 chip (carboxy methyl dextran coated chip) via primary amines, using standard amine coupling chemistry and kit provided by Biacore. Binding was measured by flowing BMP2 or BMP4 in HBS-EP buffer (pH 7.4) at a concentration of 10 nM at a flow rate of 25 μl/min. The antigen-antibody association kinetics was followed for 2 minutes and the dissociation kinetics was followed for 8 minutes. The association and dissociation curves were fit to a 1:1 Langmuir binding model using BIAevaluation software (Biacore, AB). The Kd, kₒₙ and k_{off} values that were determined are shown in Table 1.

TABLE 1
Binding affinity of anti BMP-2&4 mAbs.

| | |
|---|---|
| BMP-2 | BMP-4 |

| mAb | K_{d} (nM) | kₒₙ (1/Ms) | k_{off} (1/s) | mAb | K_{d} (nM) | kₒₙ (1/Ms) | k_{off} (1/s) |
|---|---|---|---|---|---|---|---|
| 1F6 | 0.02 | 4.65 × 10⁶ | 8.49 × 10⁻⁵ | 1F6 | 0.0003 | 4.97 × 10⁶ | 1.27 × 10⁻⁶ |
| 11F2 | 0.01 | 2.83 × 10⁶ | 3.09 × 10⁻⁵ | 11F2 | 0.0007 | 4.53 × 10⁴ | 3.04 × 10⁻⁸ |
| 16B7 | 0.08 | 3.70 × 10⁶ | 2.82 × 10⁻⁴ | 16B7 | 0.02 | 2.86 × 10⁶ | 6.12 × 10⁻⁵ |
| 12E3 | 0.02 | 3.39 × 10⁶ | 8.26 × 10⁻⁵ | 12E3 | 0.28 | 1.75 × 10⁶ | 4.82 × 10⁻⁴ |
| 10F6 | 0.19 | 2.04 × 10⁶ | 3.85 × 10⁻⁴ | 10F6 | 0.75 | 2.61 × 10⁶ | 1.97 × 10⁻³ |
| 6H4 | 0.10 | 1.42 × 10⁷ | 1.98 × 10⁻⁴ | 6H4 | 0.30 | 2.00 × 10⁶ | 4.97 × 10⁻⁴ |
| 7D6 | 0.28 | 4.00 × 10⁶ | 1.14 × 10⁻³ | 7D6 | 0.42 | 2.84 × 10⁶ | 1.20 × 10⁻³ |
| 8B3 | 0.19 | 3.02 × 10⁶ | 5.82 × 10⁻⁴ | 8B3 | 0.18 | 4.69 × 10⁶ | 8.27 × 10⁻⁴ |
| 15F3 | 0.03 | 7.96 × 10⁶ | 2.70 × 10⁻⁴ | 15F3 | 0.18 | 3.14 × 10⁶ | 5.60 × 10⁻⁴ |
| 33F7 | 0.12 | 5.71 × 10⁶ | 6.54 × 10⁻⁴ | 33F7 | 0.38 | 3.65 × 10⁶ | 1.40 × 10⁻³ |

### Example 6

### Cross-Reactivity of the Anti-BMP2/4 Monoclonal Antibodies with a Panel of BMPs

The anti-BMP2/4 monoclonal antibodies were characterized for cross-reactivity across the BMP family by measuring their binding affinities with BMP-3, 5, 6, 7 and 8b as well as with GDF-5 and 7 by Biacore analysis. The BMPs and GDFs were covalently linked to CM5 chips (carboxy methyl dextran coated chip) via primary amines, using the standard amine coupling chemistry and kit provided by Biacore. Binding was measured by flowing the antibodies in HBS-EP buffer (pH7.4) at a concentration of 20 ug/ml at a flow rate of 20 μl/min. The antigen-antibody association kinetics was followed for 4 minutes and the dissociation kinetics was followed for 6 minutes. The association and dissociation curves were fit to a 1:1 Langmuir binding model using BIAevaluation software (Biacore, AB). The Kd values that were determined are shown in Table 2.

TABLE 2
Cross reactivity of anti BMP-2&4 human monoclonal antibodies against a
panel of BMP family members.

| | BMP2 | BMP4 | BMP5 | BMP6 | BMP7 | BMP8b | BMP3 | GDF5 | GDF7 |
|---|---|---|---|---|---|---|---|---|---|
| 1F6 | 0.7 | 1.0 | 124 | 85800 | 71.7 | no | no | 17.7 | 8.8 |
| 11F2 | 0.6 | 0.4 | 26.3 | 77.0 | 20.0 | no | 104 | 16.3 | 3.5 |
| 16B7 | 0.5 | 0.5 | 18.1 | 30.0 | 8.9 | no | no | 80.0 | 2.8 |
| 12E3 | 1.4 | 2.2 | 132 | no | 106 | no | no | no | no |
| 10F6 | 17.5 | 76 | 20.1 | 103 | 18.8 | 195 | no | no | no |
| 6H4 | 3.7 | 104 | 5.9 | 40.3 | 1.1 | 159 | 246 | 0.8 | 1.1 |
| 7D6 | 4.6 | 7.7 | no | no | no | no | no | 493 | 1810 |
| 8B3 | 4.7 | 11.2 | no | no | 155 | no | no | 90.8 | 57.5 |
| 15F3 | 4.6 | 12.0 | 289 | no | 79900 | no | no | 64.8 | 57.7 |
| 33F7 | 4.3 | 271 | no | no | 579.0 | no | no | no | no |

### Example 7

### BMP Receptor Type I & II Blocking

The ability of the anti-BMP2/4 monoclonal antibodies to block BMP4 binding to type-I and type II BMP receptors (R&D systems, Minneapolis, Minn.) was determined using Biacore.

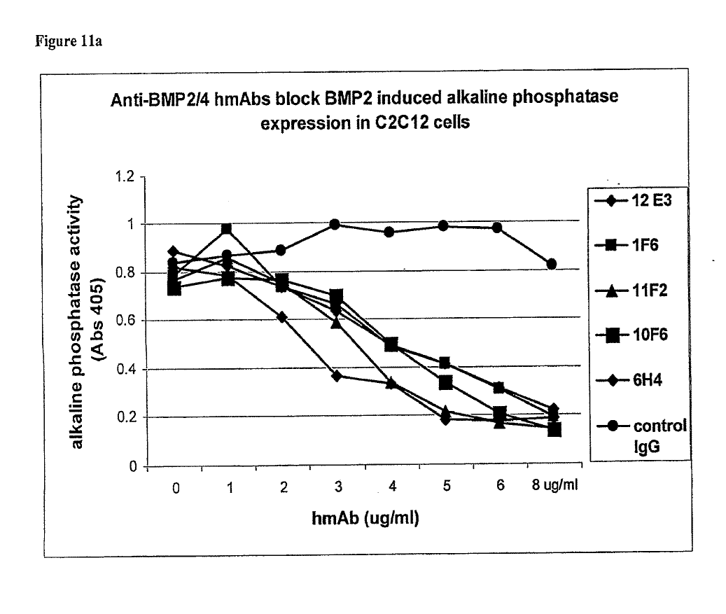

Both type-I and type-II BMP receptors were covalently linked to a CM5 chip (carboxy methyl dextran coated chip) via primary amines, using the standard amine coupling chemistry and kit provided by Biacore. Mixtures of antibody-antigen complex were flowed across the immobilized receptors. The antibody concentrations were a two-fold dilution series starting at 400 nM for type II and 200 nM for type I. The BMP4 concentration was between 3 and 10 nM. Antibodies and BMP-4 were pre-incubated for at least 1 hour prior to injection. Antibody-antigen mixtures were injected at a flow rate of 5 μl/min for 3 minutes. Antibodies that have overlapping epitopes will compete out (decreasing response with increasing antibody concentration) whereas those with distinct epitopes will simultaneously bind to the antigen (increasing response with increasing antibody concentration). This analysis showed that 1F6, 11F2, 16B7, 12E3, 10F6, 6H4, 7D6, 8B3, 15F3, and 33F7 were all able to block BMP binding to a type II receptor ranging from strong blocking to weak blocking (FIG. 10a) In addition, some of the monoclonal antibodies were also able to block type I binding whereas others only blocked type II receptor binding (FIG. 10b).

### Monoclonal Antibodies Block BMP2 Binding to Heparin

The ability of the anti-BMP2/4 monoclonal antibodies to block BMP-2 binding to heparin (Sigma) Was determined using an AlphaScreen Assay (Berthold Technologies). Biotinylated heparin (Sigma) at a concentration of 5 nM was captured by streptavidin coated donor beads (25 ug/ml) and the antibodies (5 nM) were captured using protein A coated acceptor beads. BMP/2 was titrated in a 2-fold dilution series starting from 20 nM. If the antibodies block heparin binding to BMP-2 then no complex between heparin, BMP2 and the human monoclonal antibodies would be formed and no signal observed. If the antibodies do not block heparin binding to BMP2 then a ternary complex would form and a signal would increase with increasing BMP2 concentrations. In this assay, only the 33F7 monoclonal antibody blocked heparin binding to BMP2. 33F7 binds to both heparin and BMP2 and also blocks the interaction between heparin and BMP2.

### Example 8

### Antibody Stability

### Thermostability of Anti-BMP2/4 Monoclonal Antibodies

The thermal stability of the anti-BMP2/4 monoclonal antibodies was determined by calorimetric analysis of the melting temperature of the antibodies. Calorimetric measurements of melting temperatures (Tm) were performed on a VP-Capillary DSC differential scanning microcalorimeter platform that is combined with an autosampler (MicroCal LLC, Northampton, Mass., USA). The sample cell volume was 0.144 mL. Denaturation data on the antibodies was obtained by heating the samples, at a concentration of 0.25 mg/ml, from 30 to 95° C. at a rate of 1° C./min. The antibody samples were present in phosphate-buffered saline (PBS) at pH 7.4. The same buffer was used in the reference cell to obtain the molar heat capacity by comparison. The observed thermograms were baseline corrected and normalized data analyzed based on a non-2-state model, using the software Origin v7.0. As shown in Table 3, 11F2 is the most stable anti-BMP2/4 antibody. It shows the highest Tm value for its major peak.

TABLE 3
Differential scanning calorimetry data for anti-BMP2/4 monoclonal
antibodies.

| | Tm (Major) | Tm (minor) | Tm (minor) |
|---|---|---|---|
| 11F2 | 81 | 71 | |
| 6H4 | 80 | 71 | |
| 15F3 | 80 | 72 | |
| 12E3 | 79 | 74 | |
| 1F6 | 78 | 71 | 85 |
| 8B3 | 75 | 83 | |
| 7D6 | 74 | 84 | |
| 10F6 | 73 | 68 | |
| 16B7 | 72 | 82 | |
| 33F7 | 72 | 82 | |

### Chemical Stability of Anti-BMP2/4 Monoclonal Antibodies

The stability of the anti-BMP2/4 monoclonal antibodies was compared by measuring the midpoint of their chemical denaturation by fluorescence spectroscopy. Fluorescence measurements of chemical denaturation were performed on a SPEX Fluorolog 3.22 equipped with a Micromax plate reader (SPEX, Edison, N.J.). The measurements were performed on antibody samples that had been left for 20 hours to equilibrate in 16 different concentrations of guanidinium hydrochloride in PBS buffer. The measurements were made in black, low volume, non-binding surface 384-well plates (Corning, Acton, Mass.) and required 1 μM of antibody in a well volume of 12 μL. Fluorescence was excited at 280 nm and the emission spectra were measured between 300 and 400 nm. The scan speed was 1 second per nm and slits were set to 5 nm bandpass. A buffer blank was performed using PBS and automatically subtracted from the data. Data was fitted to a two-state denaturation model using the GraphPad Prism software. As shown in Table 4, 15F3 is the most stable anti-BMP2/4 monoclonal antibody. It had the highest unfolding midpoint.

TABLE 4
The chemical denaturation of anti BMP-2&4 monoclonal antibodies
determined by fluorescence spectroscopy.

| | Unfolding Midpoint (M) |
|---|---|
| 15F3 | 2.70 |
| 10F6 | 2.66 |
| 6H4 | 2.61 |
| 8B3 | 2.53 |
| 1F6 | 2.47 |
| 7D6 | 2.41 |
| 16B7 | 2.38 |
| 12E3 | biphasic |

### Example 9

### Anti-BMP2/4 Antibodies Block BMP Cell Signaling

The effects on cell signaling by the BMP2/4 monoclonal antibodies were determined by observing alkaline phosphatase expression in C2C12 cells. To measure the ability of the monoclonal antibodies to neutralize the bioactivity of BMP2 and BMP4, C2C12 cells were plated at a density of 8,000 cells per well in a flat bottom 96 well plate in DMEM media containing 10% fetal bovine serum and 1× pen/strep and were incubated at 37° with CO₂ overnight. The next morning, the media was replaced with 100 ul of fresh medium containing monoclonal antibodies, followed by 100 μl of media containing recombinant human BMP2 protein (Medtronic) or BMP4 protein (R&D, Cat #314-BP/CF) at a concentration of 1.6 μg/ml. The plates were incubated at 37° with CO₂ for 2 days.

On the second day, the cells were assayed for alkaline phosphatase activity using a cell permeabilization method. Here, the media was removed from the wells and the cells were fixed with 100 μl of ice cold acetone/ethanol solution (50:50 v/v). The acetone/ethanol solution was removed immediately and was replaced with 100 ul of p-Nitrophenyl phosphate liquid substrate (Sigma, Cat. #N7653). The plates were kept in the dark for 3 min. at RT, and the reaction was stopped by the addition of 50 μl 3N NAOH to each well. Substrate cleavage results in a color reaction which is proportional to the amount of alkaline phosphatase in the cells. The plates were read on a SpectraMAx 340 (Molecular Devices) at a wavelength of 405 nm. The ND₅₀ for the monoclonal antibodies under these conditions were between 1-5 ug/ml.

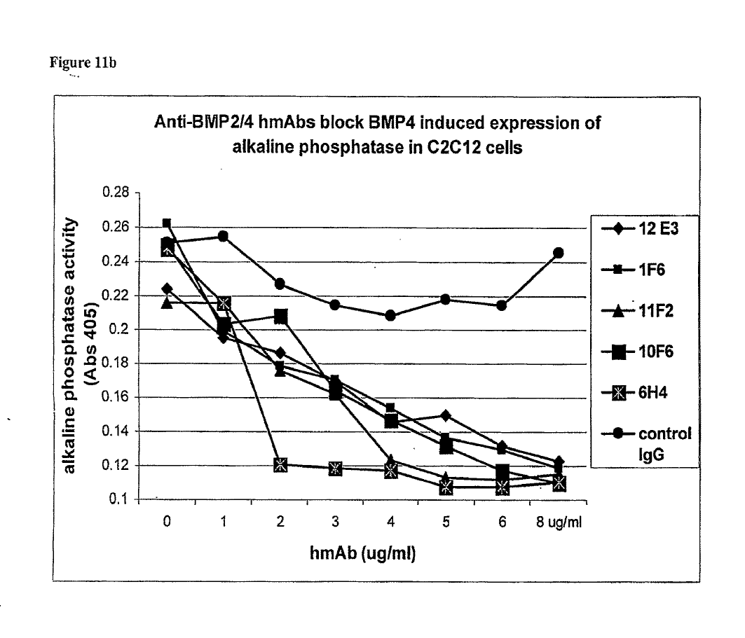

As shown in FIG. 11, expression of alkaline phosphatase by BMP2 (FIG. 11a) and BMP4 (FIG. 11b) was inhibited by the BMP2/4 monoclonal antibodies. Thus, the antibodies disclosed herein can neutralize BMP proteins.

### Example 10

### Anti-BMP2/4 Antibodies Block BMP2 Induced Heterotopic Ossification In Vivo

This example shows that the anti-BMP2/4 monoclonal antibodies block BMP2-induced heteroptopic bone formation. BMP2 induces heteroptopic bone formation when it is absorbed by a collagen gel and implanted subcutaneously into the hind limb of a mouse. The BMP2 recruits chondrocyte progenitors and vascular cells to the site of the implant to initiate bone formation. Over a 3 week period, the collagen gel becomes replaced by mature bone (Nakamura, Y. et. al. J Bone Miner Res. 2003 October; 18(10):1854-62). To show that anti-BMP2/4 antibodies can block heterotopic bone formation in vivo, mice were implanted with BMP2 infused collagen gel and were immediately treated with anti-BMP2/4 antibodies or a control irrelevant IgG (BD Pharmingen, cat #A6618M).

Absorbable collagen sponges (Helistat® Bone Graft, Integra Life Sciences cat #1690-ZZ) were infused with 96 ug/ml BMP2 (Medtronic, Infuse Bone graft) and were cut into implants with a final weight of 0.23 grams each. The BMP2-infused collagen sponges were implanted subcutaneously into the left and right hind limbs of 36 adult male C57BL6 mice. For the implantation surgery, mice were anesthetized with ketamine/xylazine according to standard protocols. In the right hind limb, the skin over the semitendinous muscle was shaved using an electric clipper and prepared with chlorhexadine scrub and alcohol. The mouse was placed in lateral recumbency. Using a scalpel or scissors, a 0.5 cm incision was made in the skin in line with the long bone. A subcutaneous implant pocket was prepared by blunt dissection. Using aseptic technique, each implant sample (collagen sponge infused with ˜25 ug BMP2) was placed in the pocket. The same procedure was repeated for implantation on the left hind limb. Wound closure was accomplished using stainless steel wound clips.

Immediately following surgery, animals were divided into 6 treatment groups (Table 5) and a single bolus injection of 300 ul of the appropriate antibody at a concentration of 1.25 mg/ml was delivered to the peritoneal cavity of each mouse. Group 1 was treated with an irrelevant control IgG. Groups 2-6 were treated with BMP2/4 neutralizing monoclonal antibodies (Table 5).

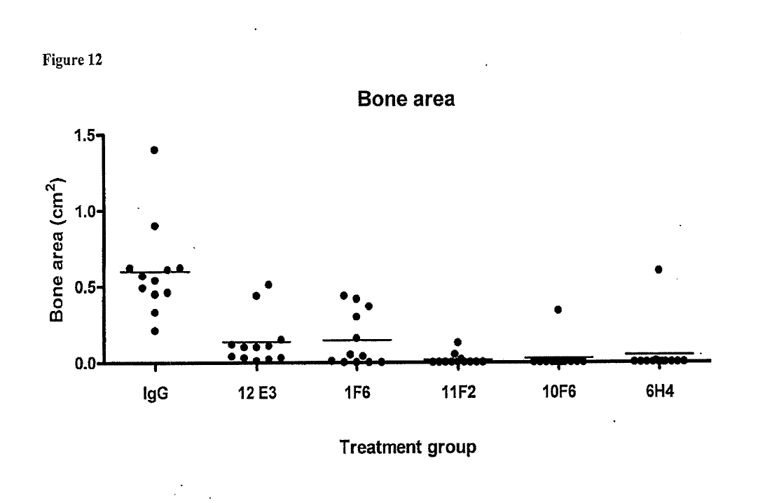

After 21 days, the implants and adjacent tissues were excised and placed in 10% neutral buffered formalin. The excised implants were subjected to densitometry scanning (PIXI, GE Lunar, Madison, Wis.). The bone mineral area (BMA) for each implant was tabulated. As shown in FIG. 12, all 5 monoclonal antibodies were effective in preventing BMP2-induced bone formation in the implants.

TABLE 5

| | Implant (left | | Concentration | |
|---|---|---|---|---|
| Group | and right) | mAb | (single IP dose) | N |
| 1 | subcutaneous | control IgG | 15 mg/Kg | 6 |
| 2 | subcutaneous | 12 E3 | 15 mg/Kg | 6 |
| 3 | subcutaneous | 1F6 | 15 mg/Kg | 6 |
| 4 | subcutaneous | 11F2 | 15 mg/Kg | 6 |
| 5 | subcutaneous | 10F6 | 15 mg/Kg | 6 |
| 6 | subcutaneous | 6H4 | 15 mg/Kg | 6 |

### Example 11

### Internalization of Anti-BMPR1A, Anti-BMPR1B, Anti-ACTR1, and/or Anti-BMPR2 Monoclonal Antibodies

This Example demonstrates methodology for testing of anti-BMPR1A, anti-BMPR1B, anti-ACTR1, and/or anti-BMPR2/4 human monoclonal antibodies for the ability to internalize into BMPR1A, BMPR1B, ACTR1, and/or BMPR2 expressing CHO cells using a Hum-Zap internalization assay. The Hum-Zap assay tests for internalization of a primary human antibody through binding of a secondary antibody with affinity for human IgG conjugated to the toxin saporin.

Antigen-expressing cells are seeded at 1.25×10⁴ cells/well in 100 μl wells overnight. The respective antigen-specific human monoclonal antibodies are added to the wells at a concentration of 10 pM. An isotype control antibody that is non-specific for any of the antigens is used as a negative control. Hum-Zap (Advanced Targeting Systems, San Diego, Calif., IT-22-25) is added at a concentration of 11 nM and plates allowed to incubate for 72 hours. Plates are then pulsed with 1.0 μCi of ³H-thymidine for 24 hours, harvested and read in a Top Count Scintillation Counter (Packard Instruments, Meriden, Conn.).

The internalization activity of saporin conjugates in antigen expressing CHO cells is measured with a dose response through a ˜500 pM to 1 pM range using human monoclonal antibodies generated as described in Example 1. A CHO parental cell line and Hu IgG-SAP are used as negative controls as a measure of background toxicity or non-specific internalization.

### Example 12

### Assessment of Cell Killing of Toxin-Conjugated Anti-BMP2, Anti-BMP4, Anti-BMPR1A, Anti-BMPR1B, Anti-ACTR1, and/or Anti-BMPR2 Antibodies on a Chondrocyte Cell Line

This Example discloses methodology for testing anti-BMP2; anti-BMP4, anti-BMPR1A, anti-BMPR1B, anti-ACTR1, and/or anti-BMPR2 monoclonal antibodies conjugated to a toxin for their ability to kill an antigen expressing chondrocyte cell line in a cell proliferation assay.

HuMAb antibodies prepared by the methodology of Example 1 may be conjugated to a toxin via a linker, such as a peptidyl, hydrazone or disulfide linker. An antigen-expressing chondrocyte or osteoblastic cell line, such as ATDC5 or MC3T3 cells, is seeded at between about 1 and 3×10⁴ cells/wells in 100 μl wells for 3 hours. An antibody-toxin conjugate is added to the wells at a starting concentration of 30 nM and titrated down at 1:3 serial dilutions. An isotype control antibody that is non-specific for antigen is used as a negative control. Plates are allowed to incubate for 69 hours. The plates are then pulsed with 1.0 μCi of ³H-thymidine for 24 hours, harvested, and read in a Top Count Scintillation Counter (Packard Instruments, Meriden, Conn.). Cell killing is shown by an antibody-toxin concentration dependent decrease in ³H-thymidine incorporation in antigen-expressing chondrocyte cells.

### Example 13

### Assessment of ADCC Activity of Anti-BMP2, Anti-BMP4, Anti-BMPR1A, Anti-BMPR1B, Anti-ACTR1, and/or Anti-BMPR2 Antibodies

This Example discloses methodology for testing of anti-BMP2, anti-BMP4, anti-BMPR1A, anti-BMPR1B, anti-ACTR1, and/or anti-BMPR2 monoclonal antibodies for the ability to kill antigen⁺ cell lines in the presence of effector cells via antibody dependent cellular cytotoxicity (ADCC) in a fluorescence cytotoxicity assay.

Human effector cells are prepared from whole blood as follows. Human peripheral blood mononuclear cells are purified from heparinized whole blood by standard Ficoll-paque separation. Cells were resuspended in RPMI1640 media containing 10% FBS and 200 U/ml of human IL-2 and incubated overnight at 37° C. The following day, the cells are collected and washed four times in culture media and resuspended at 2×10⁷ cells/ml. Target antigen⁺ cells are incubated with BATDA reagent (Perkin Elmer, Wellesley, Mass.) at 2.5 μl BATDA per 1×10⁶ target cells/mL for 20 minutes at 37° C. The target cells are washed four times, spun down and brought to a final volume of 1×10⁵ cells/ml.

The antigen⁺ cell lines are tested for antibody-specific ADCC to the human monoclonal antibodies using the Delfia fluorescence emission analysis as follows. Each target cell line (100 μl of labeled target cells) is incubated with 50 μl of effector cells and 50 μl of antibody. A target to effector ratio of 1:50 is used throughout the experiments. In all studies, a human IgG1 isotype control is used as a negative control. Following a 2000 rpm pulse spin and one hour incubation at 37° C., the supernatants are collected, quick spun again and 20 μl of supernatant is transferred to a flat bottom plate, to which 180 μl of Eu solution (Perkin Elmer, Wellesley, Mass.) is added and read in a RubyStar reader (BMG Labtech). The % lysis is calculated as follows: (sample release−spontaneous release*100)/(maximum release−spontaneous release), where the spontaneous release is the fluorescence from wells which only contain target cells and maximum release is the fluorescence from wells containing target cells and have been treated with 2% Triton-X.

### Example 14

### Treatment of In Vivo Tumor Xenograft Model Using Naked and Cytotoxin-Conjugated Anti-BMP2, Anti-BMP4, Anti-BMPR1A, Anti-BMPR1B, Anti-ACTR1, and/or Anti-BMPR2 Antibodies

This Example discloses methodology for the in vivo treatment of mice implanted with a carcinoma tumor cell with toxin-conjugated antibodies to examine the in vivo effect of the antibodies on tumor growth.

Carcinoma cells are expanded in vitro using standard laboratory procedures. Male Ncr athymic nude mice (Taconic, Hudson, N.Y.) between 6-8 weeks of age are implanted subcutaneously in the right flank with 7.5×10⁶ cells in 0.2 ml of PBS/Matrigel (1:1) per mouse. Mice are weighed and measured for tumors three dimensionally using an electronic caliper twice weekly after implantation. Tumor volumes are calculated as height×width×length. Mice with tumors averaging 110-270 mm³ are randomized into treatment groups. The mice are dosed intraperitoneally with PBS vehicle, toxin-conjugated isotype control antibody, or toxin-conjugated anti-BMP2, anti-BMP4, anti-BMPR1A, anti-BMPR1B, anti-ACTR1, and/or anti-BMPR2 HuMAb on Day 0. Examples of toxin compounds that may be conjugated to the antibodies of the current invention are described in PCT Application Publication No. WO2005/112919. The mice receiving antigen-specific human monoclonal antibodies are tested with three different toxin compounds. Mice are monitored for tumor growth for 60 days post dosing. Mice are euthanized when the tumors reach the tumor end point (2000 mm³). Suitable antigen-specific antibodies conjugated to a toxin extend the mean time to reaching the tumor end point volume (2000 mm³) and slow tumor growth progression. Thus, treatment with such an antibody-toxin conjugate has a direct in vivo inhibitory effect on tumor growth.

### Example 15

### Production of Defucosylated Human Monoclonal Antibodies

This Example discloses methodology for producing human monoclonal antibodies lacking fucosyl residues. Antibodies with reduced amounts of fucosyl residues have been demonstrated to increase the ADCC ability of the antibody. The CHO cell line Ms704-PF, which lacks the fucosyltransferase gene FUT 8 (Biowa, Inc., Princeton, N.J.), is electroporated with a vector that expresses the heavy and light chains of an antigen-specific HuMAb. Drug-resistant clones are selected by growth in Ex-Cell 325-PF CHO media (JRH Biosciences, Lenexa, KS) with 6 mM L-glutamine and 500 μg/ml G418 (Invitrogen, Carlsbad, Calif.). Clones are screened for IgG expression by standard ELISA assay. Two separate clones are produced, B8A6 and B8C11, which has production rates ranging from 1.0 to 3.8 picograms/cell/day.

### Example 16

### Assessment of ADCC Activity of Defucosylated Antibodies

This Example discloses the testing of defucosylated and non-defucosylated monoclonal antibodies for the ability to kill BMP2, BMP4, BMPR1A, BMPR1B, ACTR1, and/or BMPR2⁺ cells in the presence of effector cells via antibody dependent cellular cytotoxicity (ADCC) in a fluorescence cytotoxicity assay.

Human antigen-specific monoclonal antibodies are defucosylated as described above. Human effector cells are prepared from whole blood as follows. Human peripheral blood mononuclear cells are purified from heparinized whole blood by standard Ficoll-paque separation. The cells are resuspended in RPMI1640 media containing 10% FBS (culture media) and 200 U/ml of human IL-2 and incubated overnight at 37° C. The following day, the cells are collected and washed once in culture media and resuspended at 2×10⁷ cells/nil. Target antigen⁺ cells are incubated with BATDA reagent (Perkin Elmer, Wellesley, Mass.) at 2.5 μl BATDA per 1×10⁶ target cells/mL in culture media supplemented with 2.5 mM probenecid (assay media) for 20 minutes at 37° C. The target cells are washed four times in PBS with 20 mM HEPES and 2.5 mM probenecid, spun down and brought to a final volume of 1×10⁵ cells/ml in assay media.

A target to effector ratio of 1:100 is used throughout. A human IgG1 isotype control is used as a negative control. Following a 2100 rpm pulse spin and one hour incubation at 37° C., the supernatants are collected, quick spun again and 20 μl of supernatant is transferred to a flat bottom plate, to which 180 μl of Eu solution (Perkin Elmer, Wellesley, Mass.) is added and read in a Fusion Alpha TRF plate reader (Perkin Elmer). The % lysis is calculated as follows: (sample release−spontaneous release*100)/(maximum release−spontaneous release), where the spontaneous release is the fluorescence from wells which only contain target cells and maximum release is the fluorescence from wells containing target cells and have been treated with 3% Lysol. The antigen⁺ expressing cell line will show an antibody mediated cytotoxicity with the HuMAb antigen-specific antibodies and an increased percentage of specific lysis associated with the defucosylated form of the antigen-specific antibody. Thus, defucosylated HuMAb antibodies increase specific cytotoxicity to antigen-expressing cells.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, product descriptions, and protocols are cited throughout this application, the disclosures of which are incorporated herein by reference in their entireties for all purposes.

SUMMARY OF SEQUENCE LISTING

| SEQ ID NO: | SEQUENCE |
|---|---|
| 1 | BMP2 n.t. |
| 2 | BMP2 a.a. |
| 3 | BMP4 n.t. |
| 4 | BMP4 a.a. |
| 5 | BMPR1A n.t. |
| 6 | BMPR1A a.a. |
| 7 | BMPR1B n.t. |
| 8 | BMPR1B a.a. |
| 9 | ACTR1 n.t. |
| 10 | ACTR1 a.a. |
| 11 | BMPR2 n.t. |
| 12 | BMPR2 a.a. |
| 13 | V_{H} CDR1 a.a. 6H4 |
| 14 | V_{H} CDR1 a.a. 11F2 |
| 15 | V_{H} CDR1 a.a. 12E3 |
| 16 | V_{H} CDR2 a.a. 6H4 |
| 17 | V_{H} CDR2 a.a. 11F2 |
| 18 | V_{H} CDR2 a.a. 12E3 |
| 19 | V_{H} CDR3 a.a. 6H4 |
| 20 | V_{H} CDR3 a.a. 11F2 |
| 21 | V_{H} CDR3 a.a. 12E3 |
| 22 | V_{K} CDR1 a.a. 6H4 |
| 23 | V_{K} CDR1 a.a. 11F2 |
| 24 | V_{K} CDR1 a.a. 12E3 |
| 25 | V_{K} CDR2 a.a. 6H4 |
| 26 | V_{K} CDR2 a.a. 11F2 |
| 27 | V_{K} CDR2 a.a. 12E3 |
| 28 | V_{K} CDR3 a.a. 6H4 |
| 29 | V_{K} CDR3 a.a. 11F2 |
| 30 | V_{K} CDR3 a.a. 12E3 |
| 31 | V_{H} a.a. 6H4 |
| 32 | V_{H} a.a. 11F2 |
| 33 | V_{H} a.a. 12E3 |
| 34 | V_{K} a.a. 6H4 |
| 35 | V_{K} a.a. 11F2 |
| 36 | V_{K} a.a. 12E3 |
| 37 | V_{H} n.t. 6H4 |
| 38 | V_{H} n.t. 11F2 |
| 39 | V_{H} n.t. 12E3 |
| 40 | V_{K} n.t. 6H4 |
| 41 | V_{K} n.t. 11F2 |
| 42 | V_{K} n.t. 12E3 |
| 43 | V_{H} 4-59 germline a.a. |
| 44 | V_{H} 3-33 germline a.a. |
| 45 | D_{H} 2-2 germline a.a. |
| 46 | J_{H} JH5b germline a.a. |
| 47 | J_{H} JH6b germline a.a. |
| 48 | V_{K} A27 germline a.a. |
| 49 | V_{K} L15 germline a.a. |
| 50 | J_{K} JK4 germline a.a. |
| 51 | V_{H} 4-34 germline a.a. |
| 52 | D_{H} 3-10 germline a.a. |
| 53 | J_{H} JH1 germline a.a. |
| 54 | V_{K} L6 germline a.a. |
| 55 | J_{K} JK2 germline a.a. |
| 56 | V_{H} a.a. 10F6 |
| 57 | V_{H} a.a. 10H6 |
| 58 | V_{H} a.a. 16B7 |
| 59 | V_{H} a.a. 1F6 |
| 60 | V_{H} a.a. 7D6 |
| 61 | V_{H} a.a. 8B3 |
| 62 | V_{H} a.a. 15F3 |
| 63 | V_{H} a.a. 33F7 |
| 64 | V_{K} a.a. 10F6 |
| 65 | V_{K} a.a. 10H6 |
| 66 | V_{K} a.a. 16B7 |
| 67 | V_{K} a.a. 1F6 |
| 68 | V_{K} a.a. 7D6 |
| 69 | V_{K} a.a. 8B3 |
| 70 | V_{K} a.a. 15F3 |
| 71 | V_{K} a.a. 33F7 |
| 72 | V_{H} n.t. 10F6 |
| 73 | V_{H} n.t. 10H6 |
| 74 | V_{H} n.t. 16B7 |
| 75 | V_{K} n.t. 1F6 |
| 76 | V_{H} n.t. 7D6 |
| 77 | V_{H} n.t. 8B3 |
| 78 | V_{H} n.t. 15F3 |
| 79 | V_{H} n.t. 33F7 |
| 80 | V_{K} n.t. 10F6 |
| 81 | V_{K} n.t. 10H6 |
| 82 | V_{K} n.t. 16B7 |
| 83 | V_{K} n.t. 1F6 |
| 84 | V_{K} n.t. 7D6 |
| 85 | V_{K} n.t. 8B3 |
| 86 | V_{K} n.t. 15F3 |
| 87 | V_{K} n.t. 33F7 |
| 88 | J_{H} JH4b germline a.a. |
| 89 | J_{H} JH2 germline a.a. |
| 90 | J_{H} JH3b germline a.a. |
| 91 | V_{H} 1-69 germline a.a. |
| 92 | BMP2 epitope |
| 93 | BMP2 epitope |

We claim:

1. An isolated monoclonal antibody or an antigen binding portion thereof, an anibody fragment, or an antibody mimetic which binds an epitope on human BMP2 or BMP4 recognized by an antibody comprising a heavy chain variable region comprising the amino acid sequence set forth in SEQ ID NO:32 and a light chain variable region comprising the amino acid sequence set forth in SEQ ID NO:35.

2. The isolated antibody of claim 1, wherein said antibody is a full-length antibody of an IgG1, IgG2, IgG3, or IgG4 isotype.

3. The isolated antibody of claim 1, wherein said antibody is selected from the group consisting of: a whole antibody, an antibody fragment, a humanized antibody, a single chain antibody, an immunoconjugate, a defucosylated antibody, and a bispecific antibody.

4. The antibody fragment of claim 1, wherein the fragment is selected from the group consisting of: a UniBody, a domain antibody, and a Nanobody.

5. The antibody mimetic of claim 1, wherein the mimetic is selected from the group consisting of: an Affibody, a DARPin, an Anticalin, an Avimer, a Versabody, and a Duocalin.

6. The immunoconjugate of claim 3, wherein said immunoconjugate comprises a therapeutic agent.

7. The immunoconjugate of claim 3 wherein the therapeutic agent is a cytotoxin or a radioactive isotope.

8. The isolated antibody of claim 1, wherein said antibody binds to human BMP2 or BMP4 with a K_{D} of 5.5×10⁻⁹ M or less.

9. The isolated antibody of claim 1, wherein said antibody binds to human BMP2 or BMP4 with a K_{D} of 3×10⁻⁹ M or less.

10. The isolated antibody of claim 1, wherein said antibody binds to human BMP2 or BMP4 with a K_{D} of 2×10⁻⁹ M or less.

11. A composition comprising the isolated antibody or antigen-binding portion thereof of claim 1 and a pharmaceutically acceptable carrier.

12. An isolated nucleic acid molecule encoding the heavy or light chain of the isolated antibody or antigen-binding portion thereof of claim 1.

13. An expression vector comprising the nucleic acid molecule of claim 12.

14. A host cell comprising the expression vector of claim 13.

15. A method for preparing an anti-BMP2 or anti-BMP4 antibody, said method comprising the steps of:
a) obtaining a host cell that contains one or more nucleic acid molecules encoding the antibody of claim 1;
b) growing the host cell in a host cell culture;
c) providing host cell culture conditions wherein the one or more nucleic acid molecules are expressed; and
d) recovering the antibody from the host cell or from the host cell culture.

16. A method for treating or preventing a disease associated with abnormal bone formation and ossification, said method comprising the step of administering to a subject an anti-BMP2 or anti-BMP4 antibody, or antigen-binding portion thereof, in an amount effective to treat or prevent the disease.

17. The method of claim 14, wherein said disease is selected from the group consisting of: fibrodysplasia ossificans progressiva (FOP), progressive osseous heteroplasia (POH), spinal chord injury, intramuscular hematoma, complications from orthopedic surgery, psoriatic arthritis, osteoarthritis, ankylosing spondylitis (AS), seronegative anthropathies, skeletal hyperpstosis, otosclerosis, stapes ankylosis, bone cancer, prostate cancer, exotoses, artherosclerosis, valvular heart disease.

18. The method of claim 16, wherein said disease is a cancer selected from the group consisting of: bone cancer, prostate cancer, lung cancer, melanoma, hematopoietic cancer, renal cancer, and breast cancer.

19. An isolated monoclonal antibody or an antigen binding portion thereof, an anibody fragment, or an antibody mimetic which binds an epitope on human BMP2 or BMP4 recognized by an antibody comprising a heavy chain variable region and a light chain variable region selected from the group consisting of:
a. the heavy chain variable region amino acid sequence set forth in SEQ ID NO:33 and the light chain variable region amino acid sequence set forth in SEQ ID NO:36;
b. the heavy chain variable region amino acid sequence set forth in SEQ ID NO:34 and the light chain variable region amino acid sequence set forth in SEQ ID NO:37;
c. the heavy chain variable region amino acid sequence set forth in SEQ ID NO:56 and the light chain variable region amino acid sequence set forth in SEQ ID NO:64;
d. the heavy chain variable region amino acid sequence set forth in SEQ ID NO:57 and the light chain variable region amino acid sequence set forth in SEQ ID NO:65;
e. the heavy chain variable region amino acid sequence set forth in SEQ ID NO:58 and the light chain variable region amino acid sequence set forth in SEQ ID NO:66;
f. the heavy chain variable region amino acid sequence set forth in SEQ ID NO:59 and the light chain variable region amino acid sequence set forth in SEQ ID NO:67;
g. the heavy chain variable region amino acid sequence set forth in SEQ ID NO:60 and the light chain variable region amino acid sequence set forth in SEQ ID NO:68;
h. the heavy chain variable region amino acid sequence set forth in SEQ ID NO:61 and the light chain variable region amino acid sequence set forth in SEQ ID NO:69;
i. the heavy chain variable region amino acid sequence set forth in SEQ ID NO:62 and the light chain variable region amino acid sequence set forth in SEQ ID NO:70; and
j. the heavy chain variable region amino acid sequence set forth in SEQ ID NO:63 and the light chain variable region amino acid sequence set forth in SEQ ID NO:71.

20. The isolated antibody of claim 19, wherein said antibody is selected from the group consisting of a whole antibody, an antibody fragment, a humanized antibody, a single chain antibody, an immunoconjugate, a defucosylated antibody, and a bispecific antibody.

21. The antibody fragment of claim 19, wherein the fragment is selected from the group consisting of: a UniBody, a domain antibody, and a Nanobody.

22. The antibody mimetic of claim 19, wherein the mimetic is selected from the group consisting of: an Affibody, a DARPin, an Anticalin, an Avimer, a Versabody, and a Duocalin.

23. A composition comprising the isolated antibody or antigen binding portion thereof of claim 19 and a pharmaceutically acceptable carrier.

24. An isolated nucleic acid molecule encoding the heavy or light chain of the isolated antibody or antigen binding portion thereof of claim 19.

25. An expression vector comprising the nucleic acid molecule of claim 24.

26. A host cell comprising the expression vector of claim 25.

27. A hybridoma expressing the antibody or antigen binding portion thereof of any one of claim 1 or 19.

28. A method of making the antibody of any one of claim 1 or 19, comprising the steps of:
a. immunizing a transgenic animal comprising human immunoglobulin genes with a BMP2 or BMP4 peptide;
b. recovering B-cells from said transgenic animal;
c. making hybridomas from said B-cells;
d. selecting hybridomas that express antibodies that bind BMP2 or BMP4; and
e. recovering said antibodies that bind BMP2 or BMP4 from said selected hybridomas.

29. A method of making anti-BMP2 or anti-BMP4 antibodies, comprising the steps of:
a. immunizing a transgenic animal comprising human immunoglobulin genes with a BMP2 or BMP4 peptide;
b. recovering mRNA from the B cells of said transgenic animal;
c. converting said mRNA to cDNA;
d. expressing said cDNA in phages such that anti-BMP2 or anti-BMP4 antibodies encoded by said cDNA are presented on the surface of said phages;
e. selecting phages that present anti-BMP2 or anti-BMP4 antibodies;
f. recovering nucleic acid molecules from said selected phages that encode said anti-BMP2 or anti-BMP4 immunoglobulins;
g. expressing said recovered nucleic acid molecules in a host cell; and
recovering antibodies from said host cell that bind BMP2 or BMP4.


SEQUENCE LISTING (93 sequences)

SEQ ID NO: 1 - DNA, 3140 bp, Homo sapiens
ccacaaagggcacttggccccagggctaggagagcgaggggagagcacagccacccgcctcggcggcccgggactcggctcgactcgccggagaatgcgcccgaggacgacggggcgccagagccgcggtgctttcaactggcgagcgcgaatgggggtgcactggagtaaggcagagtgatgcgggggggcaactcgcctggcaccgagatcgccgccgtgcccttccctggacccggcatggctgccccgagccatgggccgcggcggagctagcgcggagcgcccgaccctcgacccccgagtcccggagccggccccgcgcggggccacgcgtccctcgggcgctggttcctaaggaggacgacagcaccagcttctcctttctcccttcccttccctgccccgcactcctccccctgctcgctgttgttgtgtgtcagcacttggctggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatatatatatatattttagaaaaaagaaaaaaacaaacaaacaaaaaaaccccaccccagttgacactttaatatttcccaatgaagactttatttatggaatggaatggaaaaaaaaacagctattttgaaaatatatttatatctacgaaaagaagttgggaaaacaaatattttaatcagagaattattccttaaagatttaaaatgtatttagttgtacattttatatgggttcaaccccagcacatgaagtataatggtcagatttattttgtatttatttactattataaccactttttaggaaaaaaatagctaatttgtatttatatgtaatcaaaagaagtatcgggtttgtacataattttccaaaaattgtagttgttttcagttgtgtgtatttaagatgaaaagtctacatggaaggttactctggcaaagtgcttagcacgtttgcttttttgcagtgctactgttgagttcacaagttcaagtccagaaaaaaaaagtggataatccactctgctgactttcaagattattatattattcaattctcaggaatgttgcagagtgattgtccaatccatgagaatttacatccttattaggtggaatatttggataagaaccagacattgctgatctattatagaaactctcctcctgccccttaatttacagaaagaataaagcaggatccatagaaataattaggaaaacgatgaacctgcaggaaagtgaatgatggtttgttgttcttctttcctaaattagtgatcccttcaaaggggctgatctggccaaagtattcaataaaacgtaagatttcttcattattgatattgtggtcatatatatttaaaattgatatctcgtggccctcatcaagggttggaaatttatttgtgttttacctttacctcatctgagagctctttattctccaaagaacccagttttctaactttttgcccaacacgcagcaaaattatgcacatcgtgttttctgcccaccctctgttctctgacctatcagcttgcttttctttccaaggttgtgtgtttgaacacatttctccaaatgttaaacctatttcagataataaatatcaaatctctggcatttcattctataaagtc

SEQ ID NO: 2 - protein, 392 aa, Homo sapiens [sequence omitted]

SEQ ID NO: 3 - DNA, 1779 bp, Homo sapiens
gaaggaagtggcgggggaaggagtgtggtggtggtttaaaaaataagggaagccgaggcgagagagacgcagacgcagaggtcgagcgcaggccgaaagctgttcaccgttttctcgactccggggagccattccgtagtgccatcccgagcaacgcactgctgcagcttccctgagcctttccagcaagtttgttcaagattggctgtcaagaatcatggactgttattatatgccttgttttctgtcaagacaccatgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctcctgcgggacttcgaggcgacacttctgcagatgtttgggctgcgccgccgcccgcagcctagcaagagtgccgtcattccggactacatgcgggatctttaccggcttcagtctggggaggaggaggaagagcagatccacagcactggtcttgagtatcctgagcgcccggccagccgggccaacaccgtgaggagcttccaccacgaagaacatctggagaacatcccagggaccagtgaaaactctgcttttcgtttcctctttaacctcagcagcatccctgagaacgaggcgatctcctctgcagagcttcggctcttccgggagcaggtggaccagggccctgattgggaaaggggcttccaccgtataaacatttatgaggttatgaagcccccagcagaagtggtgcctgggcacctcatcacacgactactggacacgagactggtccaccacaatgtgacacggtgggaaacttttgatgtgagccctgcggtccttcgctggacccgggagaagcagccaaactatgggctagccattgaggtgactcacctccatcagactcggacccaccagggccagcatgtcaggattagccgatcgttacctcaagggagtgggaattgggcccagctccggcccctcctggtcacctttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctgagatcaggcagtccttgaggatagacagatatacacaccacacacacacaccacatacaccacacacacacgttcccatccactcacccacacactacacagactgcttccttatagctggacttttatttaaaaaaaaaaaaaaaaaaatggaaaaaatccctaaacattcaccttgaccttatttatgactttacgtgcaaatgttttgaccatattgatcatatattttgacaaaatatatttataactacgtattaaaagaaaaaaataaaatgagtcattattttaaaggt

SEQ ID NO: 4 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEAISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 5 - DNA, 3631 bp, Homo sapiens
gcggccgctgcagagattggaatccgcctgccgggcttggcgaaggagaagggaggaggcaggagcgaggagggaggagggccaagggcgggcaggaaggcttaggctcggcgcgtccgtccgcgcgcggcgaagatcgcacggcccgatcgaggggcgaccgggtcggggccgctgcacgccaagggcgaaggccgattcgggccccacttcgccccggcggctcgccgcgcccacccgctccgcgccgagggctggaggatgcgttccctggggtccggacttatgaaaatatgcatcagtttaatactgtcttggaattcatgagatggaagcataggtcaaagctgtttggagaaaatcagaagtacagttttatctagccacatcttggaggagtcgtaagaaagcagtgggagttgaagtcattgtcaagtgcttgcgatcttttacaagaaaatctcactgaatgatagtcatttaaattggtgaagtagcaagaccaattattaaaggtgacagtacacaggaaacattacaattgaacaatgcctcagctatacatttacatcagattattgggagcctatttgttcatcatttctcgtgttcaaggacagaatctggatagtatgcttcatggcactgggatgaaatcagactccgaccagaaaaagtcagaaaatggagtaaccttagcaccagaggataccttgccttttttaaagtgctattgctcagggcactgtccagatgatgctattaataacacatgcataactaatggacattgctttgccatcatagaagaagatgaccagggagaaaccacattagcttcagggtgtatgaaatatgaaggatctgattttcagtgcaaagattctccaaaagcccagctacgccggacaatagaatgttgtcggaccaatttatgtaaccagtatttgcaacccacactgccccctgttgtcataggtccgttttttgatggcagcattcgatggctggttttgctcatttctatggctgtctgcataattgctatgatcatcttctccagctgcttttgttacaaacattattgcaagagcatctcaagcagacgtcgttacaatcgtgatttggaacaggatgaagcatttattccagttggagaatcactaaaagaccttattgaccagtcacaaagttctggtagtgggtctggactacctttattggttcagcgaactattgccaaacagattcagatggtccggcaagttggtaaaggccgatatggagaagtatggatgggcaaatggcgtggcgaaaaagtggcggtgaaagtattctttaccactgaagaagccagctggtttcgagaaacagaaatctaccaaactgtgctaatgcgccatgaaaacatacttggtttcatagcggcagacattaaaggtacaggttcctggactcagctctatttgattactgattaccatgaaaatggatctctctatgacttcctgaaatgtgctacactggacaccagagccctgcttaaattggcttattcagctgcctgtggtctgtgccacctgcacacagaaatttatggcacccaaggaaagcccgcaattgctcatcgagacctaaagagcaaaaacatcctcatcaagaaaaatgggagttgctgcattgctgacctgggccttgctgttaaattcaacagtgacacaaatgaagttgatgtgcccttgaataccagggtgggcaccaaacgctacatggctcccgaagtgctggacgaaagcctgaacaaaaaccacttccagccctacatcatggctgacatctacagcttcggcctaatcatttgggagatggctcgtcgttgtatcacaggagggatcgtggaagaataccaattgccatattacaacatggtaccgagtgatccgtcatacgaagatatgcgtgaggttgtgtgtgtcaaacgtttgcggccaattgtgtctaatcggtggaacagtgatgaatgtctacgagcagttttgaagctaatgtcagaatgctgggcccacaatccagcctccagactcacagcattgagaattaagaagacgcttgccaagatggttgaatcccaagatgtaaaaatctgatggttaaaccatcggaggagaaactctagactgcaagaactgtttttacccatggcatgggtggaattagagtggaataaggatgttaacttggttctcagactctttcttcactacgtgttcacaggctgctaatattaaacctttcagtactcttattaggatacaagctgggaacttctaaacacttcattctttatatatggacagctttattttaaatgtggtttttgatgcctttttttaagtgggtttttatgaactgcatcaagacttcaatcctgattagtgtctccagtcaagctctgggtactgaattgcctgttcataaaacggtgctttctgtgaaagccttaagaagataaatgagcgcagcagagatggagaaatagactttgccttttacctgagacattcagttcgtttgtattctacctttgtaaaacagcctatagatgatgatgtgtttgggatactgcttattttatgatagtttgtcctgtgtccttagtgatgtgtgtgtgtctccatgcacatgcacgccgggattcctctgctgccatttgaattagaagaaaataatttatatgcatgcacaggaagatattggtggccggtggttttgtgctttaaaaatgcaatatctgaccaagattcgccaatctcatacaagccatttactttgcaagtgagatagcttccccaccagctttattttttaacatgaaagctgatgccaaggccaaaagaagtttaaagcatctgtaaatttggactgttttccttcaaccaccattttttttgtggttattatttttgtcacggaaagcatcctctccaaagttggagcttctattgccatgaaccatgcttacaaagaaagcacttcttattgaagtgaattcctgcatttgatagcaatgtaagtgcctataaccatgttctatattctttattctcagtaacttttaaaagggaagttatttatattttgtgtataatgtgctttatttgcaaatcacccactcctttacaaccatactttatatatgtacatacattcatactgtagaaaccagctcatgtgtacctcatatcccatccttaagagaagaaatgttataaagtagaactaaatataaattttcagaattaatgcattcaaagtaatatatcaaatccaggactttgttaacttcaggtaaaaacttcattagggtaatatcatctcaattttttcaaatgaaaggattctctaattagaaatttatatgtcagagctgttataaatttatcaactgtcaaatatgttctggacagctaaatcatttgagatttttggttttttgatttctattccctaacttgtgaagacaatgaaaaatcaggcagaaatatttagtatctagtcagtatctgtagctacactgtataactgttcttcaataaaatggttcatattttatagaaaaaaaaaaaaaaaa

SEQ ID NO: 6 - protein, 532 aa, Homo sapiens
MPQLYIYIRLLGAYLFIISRVQGQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRWLVLLISMAVCIIAMIIFSSCFCYKHYCKSISSRRRYNRDLEQDEAFIPVGESLKDLIDQSQSSGSGSGLPLLVQRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDVPLNTRVGTKRYMAPEVLDESLNKNHFQPYIMADIYSFGLIIWEMARRCITGGIVEEYQLPYYNMVPSDPSYEDMREVVCVKRLRPIVSNRWNSDECLRAVLKLMSECWAHNPASRLTALRIKKTLAKMVESQDVKI

SEQ ID NO: 7 - DNA, 2032 bp, Homo sapiens
cgcggggcgcggagtcggcggggcctcgcgggacgcgggcagtgcggagaccgcggcgctgaggacgcgggagccgggagcgcacgcgcggggtggagttcagcctactctttcttagatgtgaaaggaaaggaagatcatttcatgccttgttgataaaggttcagacttctgctgattcataaccatttggctctgagctatgacaagagaggaaacaaaaagttaaacttacaagcctgccataagtgagaagcaaacttccttgataacatgcttttgcgaagtgcaggaaaattaaatgtgggcaccaagaaagaggatggtgagagtacagcccccaccccccgtccaaaggtcttgcgttgtaaatgccaccaccattgtccagaagactcagtcaacaatatttgcagcacagacggatattgtttcacgatgatagaagaggatgactctgggttgcctgtggtcacttctggttgcctaggactagaaggctcagattttcagtgtcgggacactcccattcctcatcaaagaagatcaattgaatgctgcacagaaaggaacgaatgtaataaagacctacaccctacactgcctccattgaaaaacagagattttgttgatggacctatacaccacagggctttacttatatctgtgactgtctgtagtttgctcttggtccttatcatattattttgttacttccggtataaaagacaagaaaccagacctcgatacagcattgggttagaacaggatgaaacttacattcctcctggagaatccctgagagacttaattgagcagtctcagagctcaggaagtggatcaggcctccctctgctggtccaaaggactatagctaagcagattcagatggtgaaacagattggaaaaggtcgctatggggaagtttggatgggaaagtggcgtggcgaaaaggtagctgtgaaagtgttcttcaccacagaggaagccagctggttcagagagacagaaatatatcagacagtgttgatgaggcatgaaaacattttgggtttcattgctgcagatatcaaagggacagggtcctggacccagttgtacctaatcacagactatcatgaaaatggttccctttatgattatctgaagtccaccaccctagacgctaaatcaatgctgaagttagcctactcttctgtcagtggcttatgtcatttacacacagaaatctttagtactcaaggcaaaccagcaattgcccatcgagatctgaaaagtaaaaacattctggtgaagaaaaatggaacttgctgtattgctgacctgggcctggctgttaaatttattagtgatacaaatgaagttgacataccacctaacactcgagttggcaccaaacgctatatgcctccagaagtgttggacgagagcttgaacagaaatcacttccagtcttacatcatggctgacatgtatagttttggcctcatcctttgggaggttgctaggagatgtgtatcaggaggtatagtggaagaataccagcttccttatcatgacctagtgcccagtgacccctcttatgaggacatgagggagattgtgtgcatcaagaagttacgcccctcattcccaaaccggtggagcagtgatgagtgtctaaggcagatgggaaaactcatgacagaatgctgggctcacaatcctgcatcaaggctgacagccctgcgggttaagaaaacacttgccaaaatgtcagagtcccaggacattaaactctgataggagaggaaaagtaagcatctctgcagaaagccaacaggtactcttctgtttgtgggcagagcaaaagacatcaaataagcatccacagtacaagccttgaacatcgtcctgcttcccagtgggttcagacctcacctttcagggagcgacctgggcaaagacagagaagctcccagaaggagagattgatccgtgtctgtttgtaggcggagaaaccgttgggtaacttgttcaagatatgatgcat

SEQ ID NO: 8 - protein, 502 aa, Homo sapiens
MLLRSAGKLNVGTKKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHRALLISVTVCSLLLVLIILFCYFRYKRQETRPRYSIGLEQDETYIPPGESLRDLIEQSQSSGSGSGLPLLVQRTIAKQIQMVKQIGKGRYGEVWMGKWRGEKVAVKVFFTTEEASWFRETEIYQTVLMRHENILGFIAADIKGTGSWTQLYLITDYHENGSLYDYLKSTTLDAKSMLKLAYSSVSGLCHLHTEIFSTQGKPAIAHRDLKSKNILVKKNGTCCIADLGLAVKFISDTNEVDIPPNTRVGTKRYMPPEVLDESLNRNHFQSYIMADMYSFGLILWEVARRCVSGGIVEEYQLPYHDLVPSDPSYEDMREIVCIKKLRPSFPNRWSSDECLRQMGKLMTECWAHNPASRLTALRVKKTLAKMSESQDIKL

SEQ ID NO: 9 - DNA, 3062 bp, Homo sapiens
gaagagatgtgggcctctggggccgctggattcagtaacttccgtcgggttctagactggctcggctctgtccagtttgtgccagatagtctcccaccccctccccacccctcctttcccctggagatttgaacgctgcttgcatgggagaaaagctacttagagaagaaaacgttccacttagtaacagaagaaaagtcttggttaaaaagttgtcatgaatttggcttttggagagaggcagcaagcctggagcattggtaagcgtcacactgccaaagtgagagctgctggagaactcataatcccaggaacgcctcttctactctccgagtaccccagtgaccagagtgagagaagctctgaacgagggcacgcggcttgaaggactgtgggcagatgtgaccaagagcctgcattaagttgtacaatggtagatggagtgatgattcttcctgtgcttatcatgattgctctcccctcccctagtatggaagatgagaagcccaaggtcaaccccaaactctacatgtgtgtgtgtgaaggtctctcctgcggtaatgaggaccactgtgaaggccagcagtgcttttcctcactgagcatcaacgatggcttccacgtctaccagaaaggctgcttccaggtttatgagcagggaaagatgacctgtaagaccccgccgtcccctggccaagctgtggagtgctgccaaggggactggtgtaacaggaacatcacggcccagctgcccactaaaggaaaatccttccctggaacacagaatttccacttggaggttggcctcattattctctctgtagtgttcgcagtatgtcttttagcctgcctgctgggagttgctctccgaaaatttaaaaggcgcaaccaagaacgcctcaatccccgagacgtggagtatggcactatcgaagggctcatcaccaccaatgttggagacagcactttagcagatttattggatcattcgtgtacatcaggaagtggctctggtcttccttttctggtacaaagaacagtggctcgccagattacactgttggagtgtgtcgggaaaggcaggtatggtgaggtgtggaggggcagctggcaaggggaaaatgttgccgtgaagatcttctcctcccgtgatgagaagtcatggttcagggaaacggaattgtacaacactgtgatgctgaggcatgaaaatatcttaggtttcattgcttcagacatgacatcaagacactccagtacccagctgtggttaattacacattatcatgaaatgggatcgttgtacgactatcttcagcttactactctggatacagttagctgccttcgaatagtgctgtccatagctagtggtcttgcacatttgcacatagagatatttgggacccaagggaaaccagccattgcccatcgagatttaaagagcaaaaatattctggttaagaagaatggacagtgttgcatagcagatttgggcctggcagtcatgcattcccagagcaccaatcagcttgatgtggggaacaatccccgtgtgggcaccaagcgctacatggcccccgaagttctagatgaaaccatccaggtggattgtttcgattcttataaaagggtcgatatttgggcctttggacttgttttgtgggaagtggccaggcggatggtgagcaatggtatagtggaggattacaagccaccgttctacgatgtggttcccaatgacccaagttttgaagatatgaggaaggtagtctgtgtggatcaacaaaggccaaacatacccaacagatggttctcagacccgacattaacctctctggccaagctaatgaaagaatgctggtatcaaaatccatccgcaagactcacagcactgcgtatcaaaaagactttgaccaaaattgataattccctcgacaaattgaaaactgactgttgacattttcatagtgtcaagaaggaagatttgacgttgttgtcattgtccagctgggacctaatgctggcctgactggttgtcagaatggaatccatctgtctccctccccaaatggctgctttgacaaggcagacgtcgtacccagccatgtgttggggagacatcaaaaccaccctaacctcgctcgatgactgtgaactgggcatttcacgaactgttcacactgcagagactaatgttggacagacactgttgcaaaggtagggactggaggaacacagagaaatcctaaaagagatctgggcattaagtcagtggctttgcatagctttcacaagtctcctagacactccccacgggaaactcaaggaggtggtgaatttttaatcagcaatattgcctgtgcttctcttctttattgcactaggaattctttgcattccttacttgcactgttactcttaattttaaagacccaacttgccaaaatgttggctgcgtactccactggtctgtctttggataataggaattcaatttggcaaaacaaaatgtaatgtcagactttgctgcattttacacatgtgctgatgtttacaatgatgccgaacattaggaattgtttatacacaactttgcaaattatttattacttgtgcacttagtagtttttacaaaactgctttgtgcatatgttaaagcttatttttatgtggtcttatgattttattacagaaatgtttttaacactatactctaaaatggacattttcttttattatcagttaaaatcacattttaagtgcttcacatttgtatgtgtgtagactgtaactttttttcagttcatatgcagaacgtatttagccattacccacgtgacaccaccgaatatattactgatttagaagcaaagatttcagtagaattttagtcctgaacgctacggggaaaatgcattttcttcagaattatccattacgtgcatttaaactctgccagaaaaaaataactattttgttttaatctactttttgtatttagtagttatttgtataaattaaataaactgttttcaagtcaaaaaaaaaaaaaaaaaaaa

SEQ ID NO: 10 - protein, 509 aa, Homo sapiens
MVDGVMILPVLIMIALPSPSMEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEVGLIILSVVFAVCLLACLLGVALRKFKRRNQERLNPRDVEYGTIEGLITTNVGDSTLADLLDHSCTSGSGSGLPFLVQRTVARQITLLECVGKGRYGEVWRGSWQGENVAVKIFSSRDEKSWFRETELYNTVMLRHENILGFIASDMTSRHSSTQLWLITHYHEMGSLYDYLQLTTLDTVSCLRIVLSIASGLAHLHIEIFGTQGKPAIAHRDLKSKNILVKKNGQCCIADLGLAVMHSQSTNQLDVGNNPRVGTKRYMAPEVLDETIQVDCFDSYKRVDIWAFGLVLWEVARRMVSNGIVEDYKPPFYDVVPNDPSFEDMRKVVCVDQQRPNIPNRWFSDPTLTSLAKLMKECWYQNPSARLTALRIKKTLTKIDNSLDKLKTDC

SEQ ID NO: 11 - DNA, 11449 bp, Homo sapiens
gcgaaacttaaggaatcctgccttcccggagccgcgggcgatgcgactagggctgccgggcgccgccgccgcccgtccggcttcgtccttcccggcagtcgggaactagttctgaccctcgccccccgaccccggatcgaatccccgccctccgcaccctggatatgttttctcccagacctggatatttttttgatatcgtgaaactacgagggaaataatttgggggatttcttcttggctccctgctttccccacagacatgccttccgtttggagggccgcggcaccccgtccgaggcgaaggaacccccccagccgcgagggagagaaatgaagggaatttctgcagcggcatgaaagctctgcagctaggtcctctcatcagccatttgtcctttcaaactgtattgtgatacgggcaggatcagtccacgggagagaagacgagcctcccggctgtttctccgccggtctacttcccatatttcttttctttgccctcctgattcttggctggcccagggatgacttcctcgctgcagcggccctggcgggtgccctggctaccatggaccatcctgctggtcagcactgcggctgcttcgcagaatcaagaacggctatgtgcgtttaaagatccgtatcagcaagaccttgggataggtgagagtagaatctctcatgaaaatgggacaatattatgctcgaaaggtagcacctgctatggcctttgggagaaatcaaaaggggacataaatcttgtaaaacaaggatgttggtctcacattggagatccccaagagtgtcactatgaagaatgtgtagtaactaccactcctccctcaattcagaatggaacataccgtttctgctgttgtagcacagatttatgtaatgtcaactttactgagaattttccacctcctgacacaacaccactcagtccacctcattcatttaaccgagatgagacaataatcattgctttggcatcagtctctgtattagctgttttgatagttgccttatgctttggatacagaatgttgacaggagaccgtaaacaaggtcttcacagtatgaacatgatggaggcagcagcatccgaaccctctcttgatctagataatctgaaactgttggagctgattggccgaggtcgatatggagcagtatataaaggctccttggatgagcgtccagttgctgtaaaagtgttttcctttgcaaaccgtcagaattttatcaacgaaaagaacatttacagagtgcctttgatggaacatgacaacattgcccgctttatagttggagatgagagagtcactgcagatggacgcatggaatatttgcttgtgatggagtactatcccaatggatctttatgcaagtatttaagtctccacacaagtgactgggtaagctcttgccgtcttgctcattctgttactagaggactggcttatcttcacacagaattaccacgaggagatcattataaacctgcaatttcccatcgagatttaaacagcagaaatgtcctagtgaaaaatgatggaacctgtgttattagtgactttggactgtccatgaggctgactggaaatagactggtgcgcccaggggaggaagataatgcagccataagcgaggttggcactatcagatatatggcaccagaagtgctagaaggagctgtgaacttgagggactgtgaatcagctttgaaacaagtagacatgtatgctcttggactaatctattgggagatatttatgagatgtacagacctcttcccaggggaatccgtaccagagtaccagatggcttttcagacagaggttggaaaccatcccacttttgaggatatgcaggttctcgtgtctagggaaaaacagagacccaagttcccagaagcctggaaagaaaatagcctggcagtgaggtcactcaaggagacaatcgaagactgttgggaccaggatgcagaggctcggcttactgcacagtgtgctgaggaaaggatggctgaacttatgatgatttgggaaagaaacaaatctgtgagcccaacagtcaatccaatgtctactgctatgcagaatgaacgcaacctgtcacataataggcgtgtgccaaaaattggtccttatccagattattcttcctcctcatacattgaagactctatccatcatactgacagcatcgtgaagaatatttcctctgagcattctatgtccagcacacctttgactataggggaaaaaaaccgaaattcaattaactatgaacgacagcaagcacaagctcgaatccccagccctgaaacaagtgtcaccagcctctccaccaacacaacaaccacaaacaccacaggactcacgccaagtactggcatgactactatatctgagatgccatacccagatgaaacaaatctgcataccacaaatgttgcacagtcaattgggccaacccctgtctgcttacagctgacagaagaagacttggaaaccaacaagctagacccaaaagaagttgataagaacctcaaggaaagctctgatgagaatctcatggagcactctcttaaacagttcagtggcccagacccactgagcagtactagttctagcttgctttacccactcataaaacttgcagtagaagcaactggacagcaggacttcacacagactgcaaatggccaagcatgtttgattcctgatgttctgcctactcagatctatcctctccccaagcagcagaaccttcccaagagacctactagtttgcctttgaacaccaaaaattcaacaaaagagccccggctaaaatttggcagcaagcacaaatcaaacttgaaacaagtcgaaactggagttgccaagatgaatacaatcaatgcagcagaacctcatgtggtgacagtcaccatgaatggtgtggcaggtagaaaccacagtgttaactcccatgctgccacaacccaatatgccaatgggacagtactatctggccaaacaaccaacatagtgacacatagggcccaagaaatgttgcagaatcagtttattggtgaggacacccggctgaatattaattccagtcctgatgagcatgagcctttactgagacgagagcaacaagctggccatgatgaaggtgttctggatcgtcttgtggacaggagggaacggccactagaaggtggccgaactaattccaataacaacaacagcaatccatgttcagaacaagatgttcttgcacagggtgttccaagcacagcagcagatcctgggccatcaaagcccagaagagcacagaggcctaattctctggatctttcagccacaaatgtcctggatggcagcagtatacagataggtgagtcaacacaagatggcaaatcaggatcaggtgaaaagatcaagaaacgtgtgaaaactccctattctcttaagcggtggcgcccctccacctgggtcatctccactgaatcgctggactgtgaagtcaacaataatggcagtaacagggcagttcattccaaatccagcactgctgtttaccttgcagaaggaggcactgctacaaccatggtgtctaaagatataggaatgaactgtctgtgaaatgttttcaagcctatggagtgaaattattttttgcatcatttaaacatgcagaagatgtttaaaaataaaaaaaaaactgctttatcctcctgtcagcaccccctcccacccctgcaacaaagacttgctttaaatagatttcagctatgcagaaaaatttagcttatgcttccatatttttaaattttgttttttaagttttgcacttttgtttagtctcgctaaagttatatttgtctgttatgaccacagagttatatgtgtgtgtatcaaaagtggtctcaaaatatttttttaagaaaaaaagcaaaaacaatgtattgctgataatcagtttggaccagtttcttaaggtcattaaaacagaagcaaattaagacaggtttgactgcagtggtgtctggtatccatgttttatttctgggcacaagctagtttttatgttgatacgttcctgaacatattatcttgttggacatcttttctcttgtgttttgtttgaatgtgcaatagtttataggccacaaataagctttcttgtaagctctcttcctaacagggcacatattcttccataatataaacacttttctgccccatctcccatacttttgaaggtcagttctatgacagtgaattttgcacaggagaagcagctacctgatttcttactttctctctccttatcatggagaatacagaaacattgtctgaaagggctctaaagaaggaactaccaaaacctgacttgaaatgccatttcttttaaccttccaaatcctaaatgtttccttcaaggcatcttaataaacttatttgcttctggttttgggagttcataagagagaatagaacaaaatacaggacatcaaatattagccatttcccattttattttatttttctatgtaggttcatgttccatgttcatttatttaagaaatacatttttattggtaagcttatagagctacacttatggaatttttaagtaggtaaataaatggttaagacaaaatagtgttatagccttcattctctgaataggccatctttgactcataaaattacccttactgtttattataacttcagaagtaatttatagttctgaacctatagtatcttttaccctgttcccaagcaaagactggtgactttatctgaaaatgattcctcttcccatgacctaaaacactgtgaggaaaaatcattcaagtggcatgccaagtccctatgaaggaagggctgctatcaaacctaccttttttgagcaaactgagactaaacttctctcttttcaaaattgtgttatcttccttaatcctattttcataatttttccttttgccagtttttcacattatctttgatatgtgagcaacatttattatttacattagagtataccttttagtaataaaatgacttgaaatcatattatttttaaaagccctttgcttctttcattacttataatctcctctaaaacaacctctgcatgttttttttaaataaagcactttctgtcaaataatggacttttttctaaacagaaattattttctattaatttgcaaactgatgatttcacttttttttacttttttttcgattatcagagtacttagtaaatgttatatagtttagttctaagatagttccagggattaaaaggttaagaggaaaacacaaatcaccaaatttctgatttatgtttttatctcctgaacaatattttctcactcatattcctctatcttatcacttagctaaagacagcctaaatttcccaattttctgtccaaaatatttgtgatttacttgtatataagccttctcatttgccatgtgctgtgatcttacaagttagatgttactatacctaccatttattcagctggattgctgaacacagttctggattcatagaattaagaatatcttgttagtgcccaggatttccacgttttgtgttttattggcccttttctttattcagccccttaatctattttcagtcttctggcacgtaatttttttcacagttattattcttctattaacaaattatttttatctttcctagtacattttcacttagttctcttgcccttaaatatctttcacatgcattttaggatattcttttcaaatatttgtaggacaactttgaatcaaaataaattatgttccttctccaatttgaagcattgaggataaatgaccatttgaggtctaactgatcttttcctgccagaagagttatcttacgttctgctatatttgtatttgggccagttgattgtaggttgtccaacattttttaatattgggaaaattatgataaaatgctttaaaaattaatatgccagattaaaataactgaatagtttactatttcattcaagcatgtttaaaacaaataatttcctttcaccagtttttcttagtaaactcctgaaaaagtaggaaaggtggaaagtatatatcatttttataaattttaaattgtacatcagacttttaaaatctgtaatatacaagcaagcaaaattattttaaatgacttaattgtatgctaatactcatctgataataaatgcttcttaaagttgacatttaactgctatcacaaagttttatatgtagaaaagtggggtccttttgaataaaagatcattcaactaaaaatattaaaatttatttcactggatggtaatgtaaccttaaaagcatcataataggtaaagtctaatattagttcccttaacaaaatcctaactgtataccagaattaggtcactgaaagaacttgatttgaattacgtttagacaaaaatgatttaattgtaaattcttaaaactttctaaatgcataattggcaaaaaaaaaaacccactgttaccagtgtaggaagttacaagaaggcacatactgaatgctgaagtatacatatgctatttctcttaaacctcagagcaaccatatgagcattgtaattaatattcccattttacagatgaggaaactgaagctaagagaagctaagtaatatgcccaaggtccacatctagtaacagacaaagctgggatttcagtctatgtctgcctctctccacatctctttcatccataccacactgcctacatgccatatgacaggatgtgtaatgggctaacgtttatttaaaaagttcaggccaggtgcagtggcttatgcctataatccccgcactttgggaggccgagccgggtggatcatgaggtcagacgttcaacaccagcctggccaagatggtgaaaccccgtctctattaaaaatattttttaaaaattaactgagcgtggtggtgggcgcctgtagtcccagctactcaggaggctaaggcaggagaatctcttgaacccgagaggtggaggttgcagtgagctgagatcgcgccactgcactgcagcctaggcgacagagcaagactgcctcaaaaaaaaaacataaaaattcagtcactatactctggcacaattttcatttgtatatgagccaaaccacataccttaatgatttgggaagttaggcaaatatgagatatgtagacatatctatgctgattgttgcttgagaaataattactaattctagacaaaactcaatcctgtatcttccatcatgaatcttaaaatcatttcacttcactcctaacatttcttacattgcagaactaatggtaaagtaaattttacggaaaaagttaagatagctttgggaacagagacctttccctaaattgattccatagcagatttgggggaattaacaaagaatttcagtctcatcaatcctttgaatccatcttcaaaacttctgcttttaataactttagaaaatttactaatctatagaactaattgagtaggatataggaaggatacaaggatataatgtcctttttataaaagtttagtatagcttctttacatgtatccacttgttccagaaaatgtgcattggttctgaatgtgaaaatatttaaagagagaaaggaacactcaagtaagtgtgggcttcagtgggaattatcacaaaacattggcaagtatttttatttaaattattttcaaatttgacttctacagccaagtggaattggtaggctgtagctgttacactgaaatttctagtctttgtaagtgcctcctgaaagtcatttaaaatggaaaaatatttcaatgagcttttccttttttcatatttatggacatgaatattttattggagatcattaactcctagaatttgagattatatttccatacaacattttataaagttatgttgaacttactacctgttatgtgcaggttattatgtaactattcacagattgcttcatatattgctttatcttcccatctaacttcttaaagttaaaatccggacacacatgttgattatctagaccagtcattctggaaattgtaacactcccacataaaccccaggagactttttcagaatgcaatgtttctaaatgtactgttactggcagtttactctccagcatataaggttgcattttaacttttagattatgaactgtgcaaactttacccaaaactatcttgcatgattccctcctaaatatattccttgattaagtaaatctggcaaatcactgtttgagctagttacataaaatttgttatcaagagaaggcttttctacaagtttccagattaacataaagaaaagagggaatcacagggcatttaagtgcaccttcccattactttccttaaatcacctcatagttaggctgggcgcggtggctcacgcctgtaatcccagcactttgggaggccgagacggtggatcacgaggtcaggggactgagatcatcctggctaacacgatgaaacctcatctctactaaaaatacaaataattagccaggcatggtggcacgcgcctgtagtcccagctactcgggaggcagagacaggagaatcgtttgcacccgggaggcggaggttgttgcagtgagccgagatcgcgccaatgcactccagcctgggctacagagtgagactccatctcaaaaaaaaacaaaaaaaatcacctcatagtttatgtctgacttactccaaacctcagttatctgatttgtagtctgtgtcaggaaagttttcttcatatctattctgtctccctcctctttgattttaaatttttttcttttacccagtaggacaaaaaagagcagttggtcatcatccccaatattcttagtcttcagtatgcttcaggcctctcaatgaacacttaagtctcaattcttcagacaaaattgcttaagctcttctcctcagtcctattttaatgactttatatttaagaatatagaattatattttcttttatattcaaattcattactccagttaagtaatagtttgatagtttgatagaatcgagagttaagatgtttctatttgaaagtggattcaaccatcagaccaccagcaaatcggcacttaatttttgtgttatctaacattttctattgtggaattttatgattttatattctcattagttataactaaaaagccatgcacacagaattgtatatcattttgccattaaaattttttaacatattgcagcaagcttagttttatgattgagccacaaccttttacatattttttgtatgaaatattaaacactaaatgcaagattaactttcaaaagcaaaccctacattaatcaggtattatctatggacatttttgtagaccacttttgaaatacttattattttgcaacatagactggactatacaactttcatttaacttttaggtgactgatttaagttgagtgtgcatatagagaaaaacctagaaatttatctcatggcagatacatttgaaagtacttcagaagaatttatgctgtatattaaaactaggctcaaaataaatctatcgtatctttaaaagtccaattctgttattactgtgatgtttgtagtgttactattaaacattgtgaacatacacatttttaaaacaacttgaaacccattttaaaatctgggtaagagagaaggaatcttcagaacaaaatcacatcattagggtgtccagtttatgattgaatttttaagcaaattactgtatttgaaactacaacttgatttggttttcagttttaaaaggcaacatgtgggttttatccattttatttatacctttagatttcagaaacatcttcatgttttagatgcattctacagacatcatgttacttaaaaactcagggcccctttcatccctttgtacactgaaaaagttcaattgttagcaagtaagcaattagatccagttgaatatttaaagtgtttgttgcacagttcatttaatgtttcatcttatttgactttttcacatagatataatatcagatttcattaattataaaaagttgcccagttctgtaattactgaacagagggaatgactcaactaattggctacatgttgcaacaaatttaggcctttagagttgaagcactgacttaaaacgacttacatttctgttctttggtcaaatgaccatacatgatatgggacaaattgtttcattttgtttgttttttaataagggaacttggtaaagtagttcctgtcagataggattttctcaagagacaatttaacgttataaagccttctaaaagtgaactaaatattttataactttagtaatagcttggatggttttgagaaaataacctgtatttatcacattgtcaaacagaatttttctttgaatcagacaagttcaagctctaaattgatgtgctatatacttaaaatcctaggaagttatctgtaaccagtctcttgtctcaggctcttcaccttgttaccaatcctcgtaagtatgtaaaggaaacatatttttaaagaagcttaacagtaagaaaaaattactaaaagatgcaattcaaagataggtcccagtttaacactgaattgcttgacttctgtggcttttctttttctggccacatttatttatttaagcaatttttgtatgccttgttatttcatttccatagagattatattgtatcagtgtttatgtaagctggaatcatcctcagttttttgctgataatttttcaaataaagatacatggataattgtaaaatacactaactcttagggtgttgtagtagctgaaacatggagatgcgtagctgtcatgctttttctgaatggacaggagaaacataagctacggagtattcacttctgaggatgcttttccggaaaaagaaaggctagaaaatactcgcacttcctcagaaccctctttcttgttaacgggtatcttttgttggtgtgttttgctcttacattacagatagactatcatatatgactttatgaataatttcagttattttgcttttgtataagctgtctgaagccttgctatgctgtataagttgtgtttgatggatcagtgtgagtataaaataaagcaaatcacttttcttttgtattatctatggatgccactatgaaagctgacattaagccactaaagagttttctatgaataagtgtaagtaaatgctttgatatatataaacctaaataaaaagattgtattgatacagagacattggagaaggagattttaaggcagttctttaggtttaaaaaggcttgctgtaaaatggtgcgttattccgtttattaaagatcatattaatgacaata

SEQ ID NO: 12 - protein, 1038 aa, Homo sapiens
MTSSLQRPWRVPWLPWTILLVSTAAASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETIIIALASVSVLAVLIVALCFGYRMLTGDRKQGLHSMNMMEAAASEPSLDLDNLKLLELIGRGRYGAVYKGSLDERPVAVKVFSFANRQNFINEKNIYRVPLMEHDNIARFIVGDERVTADGRMEYLLVMEYYPNGSLCKYLSLHTSDWVSSCRLAHSVTRGLAYLHTELPRGDHYKPAISHRDLNSRNVLVKNDGTCVISDFGLSMRLTGNRLVRPGEEDNAAISEVGTIRYMAPEVLEGAVNLRDCESALKQVDMYALGLIYWEIFMRCTDLFPGESVPEYQMAFQTEVGNHPTFEDMQVLVSREKQRPKFPEAWKENSLAVRSLKETIEDCWDQDAEARLTAQCAEERMAELMMIWERNKSVSPTVNPMSTAMQNERNLSHNRRVPKIGPYPDYSSSSYIEDSIHHTDSIVKNISSEHSMSSTPLTIGEKNRNSINYERQQAQARIPSPETSVTSLSTNTTTTNTTGLTPSTGMTTISEMPYPDETNLHTTNVAQSIGPTPVCLQLTEEDLETNKLDPKEVDKNLKESSDENLMEHSLKQFSGPDPLSSTSSSLLYPLIKLAVEATGQQDFTQTANGQACLIPDVLPTQIYPLPKQQNLPKRPTSLPLNTKNSTKEPRLKFGSKHKSNLKQVETGVAKMNTINAAEPHVVTVTMNGVAGRNHSVNSHAATTQYANGTVLSGQTTNIVTHRAQEMLQNQFIGEDTRLNINSSPDEHEPLLRREQQAGHDEGVLDRLVDRRERPLEGGRTNSNNNNSNPCSEQDVLAQGVPSTAADPGPSKPRRAQRPNSLDLSATNVLDGSSIQIGESTQDGKSGSGEKIKKRVKTPYSLKRWRPSTWVISTESLDCEVNNNGSNRAVHSKSSTAVYLAEGGTATTMVSKDIGMNCL

SEQ ID NO: 13 - protein, 5 aa, Homo sapiens
GYYWS

SEQ ID NO: 14 - protein, 5 aa, Homo sapiens
SYYWS

SEQ ID NO: 15 - protein, 5 aa, Homo sapiens
SYGMH

SEQ ID NO: 16 - protein, 16 aa, Homo sapiens
EINHSGSTNYNPSLKS

SEQ ID NO: 17 - protein, 16 aa, Homo sapiens
YIYYRGSTHYNPSLKS

SEQ ID NO: 18 - protein, 17 aa, Homo sapiens
VIWDDGRKKYYADSVKG

SEQ ID NO: 19 - protein, 13 aa, Homo sapiens
EYYYGSESEYFQH

SEQ ID NO: 20 - protein, 13 aa, Homo sapiens
ICSSISCWGWFDP

SEQ ID NO: 21 - protein, 10 aa, Homo sapiens
EPAGVWGMDV

SEQ ID NO: 22 - protein, 11 aa, Homo sapiens
RASQSVSSYLA

SEQ ID NO: 23 - protein, 12 aa, Homo sapiens
RASQSVSSSYLA

SEQ ID NO: 24 - protein, 11 aa, Homo sapiens
RASQGISSWLA

SEQ ID NO: 25 - protein, 7 aa, Homo sapiens
DASNRAT

SEQ ID NO: 26 - protein, 7 aa, Homo sapiens
GASSRAT

SEQ ID NO: 27 - protein, 6 aa, Homo sapiens
ASSLQS

SEQ ID NO: 28 - protein, 9 aa, Homo sapiens
QQRSNWPHT

SEQ ID NO: 29 - protein, 9 aa, Homo sapiens
QQYGSSPLT

SEQ ID NO: 30 - protein, 7 aa, Homo sapiens
YNSYPLT

SEQ ID NO: 31 - protein, 121 aa, Homo sapiens
QVHLQQWGAGLLKPSETLSLTCAVYGGSFSGYYWSWIRQPPGKGLEWIGEINHSGSTNYNPSLKSRVTISVDTSKNQFSLKLSSVTAADTAVYYCAREYYYGSESEYFQHWGQGTLVTVSS

SEQ ID NO: 32 - protein, 121 aa, Homo sapiens
QVQLQESGPGLVKPSETLSLTCTVSGDSIRSYYWSWIRQPPGKGLEWIGYIYYRGSTHYNPSLKSRVTISVDTSKNQFSLKMSSVTAADTAVYYCARICSSISCWGWFDPWGQGTLVTVSS

SEQ ID NO: 33 - protein, 119 aa, Homo sapiens
QVQLVESGGGVVQPGRSLRLSCAASGFTFSSYGMHWVRQAPGKGLEWVAVIWDDGRKKYYADSVKGRFTISRDNSKNTLYLQMNSLRAEDTAVYYCAREPAGVWGMDVWGQGTTVTVSS

SEQ ID NO: 34 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPHTFGQGTKLEIK

SEQ ID NO: 35 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIK

SEQ ID NO: 36 - protein, 107 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIK

SEQ ID NO: 37 - DNA, 363 bp, Homo sapiens
  CDS (1)..(363)
caggtgcacctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagagagtattattatggttcggagagtgaatacttccagcactggggccagggcaccctggtcaccgtctcctca

SEQ ID NO: 38 - DNA, 363 bp, Homo sapiens
  CDS (1)..(363)
caggtgcagctgcaggagtcgggcccaggactggtgaagccttcggagaccctgtccctcacctgcactgtctctggtgactccatcaggagttactactggagctggatccggcagcccccagggaagggactggagtggattggatatatctattacagagggagcacccactacaacccctccctcaagagtcgagtcaccatatcagtagacacgtccaagaatcagttctccctgaagatgagctctgtgaccgctgcggacacggccgtgtattactgtgcgaggatttgtagtagtatcagctgttggggctggttcgacccctggggccagggaaccctggtcaccgtctcctca

SEQ ID NO: 39 - DNA, 357 bp, Homo sapiens
  CDS (1)..(357)
caggtgcagctggtggagtctgggggaggcgtggtccagcctgggaggtccctgagactctcctgtgcagcgtctggattcaccttcagtagttatggcatgcactgggtccgccaggctccaggcaaggggctggagtgggtggcagttatatgggatgatggaagaaagaaatactatgcagactccgtgaagggccgatttaccatctccagagacaattccaagaacacgctgtatctgcaaatgaacagcctgagagccgaggacacggctgtgtattactgtgcgagagagccggcgggggtttggggtatggacgtctggggccaagggaccacggtcaccgtctcctca

SEQ ID NO: 40 - DNA, 321 bp, Homo sapiens
  CDS (1)..(321)
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctcacacttttggccaggggaccaagctggagatcaaa

SEQ ID NO: 41 - DNA, 324 bp, Homo sapiens
  CDS (1)..(324)
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgctcactttcggcggagggaccaaggtggagatcaaa

SEQ ID NO: 42 - DNA, 321 bp, Homo sapiens
  CDS (1)..(321)
gacatccagatgacccagtctccatcctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttacccgctcactttcggcggagggaccaaggtggagatcaaa

SEQ ID NO: 43 - protein, 97 aa, Homo sapiens
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAR

SEQ ID NO: 44 - protein, 98 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAR

SEQ ID NO: 45 - protein, 10 aa, Homo sapiens
GYCSSTSCYT

SEQ ID NO: 46 - protein, 15 aa, Homo sapiens
WFDPWGQGTLVTVSS

SEQ ID NO: 47 - protein, 15 aa, Homo sapiens
GMDVWGQGTTVTVSS

SEQ ID NO: 48 - protein, 96 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 49 - protein, 95 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYP

SEQ ID NO: 50 - protein, 12 aa, Homo sapiens
LTFGGGTKVEIK

SEQ ID NO: 51 - protein, 97 aa, Homo sapiens
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCAR

SEQ ID NO: 52 - protein, 10 aa, Homo sapiens
YYYGSGSYYN

SEQ ID NO: 53 - protein, 16 aa, Homo sapiens
EYFQHWGQGTLVTVSS

SEQ ID NO: 54 - protein, 95 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWP

SEQ ID NO: 55 - protein, 11 aa, Homo sapiens
TFGQGTKLEIK

SEQ ID NO: 56 - protein, 118 aa, Homo sapiens
QVQLVESGGGVVQPGRSLRLSCAASGFTFRSYGIHWVRQAPGKGLEWVAVIWYDGSHKYYADSVKGRFTISRDNSKNTLDLQMNSLRAEDTAVYYCVRGSSNWYFDYWGQGTLVTVSS

SEQ ID NO: 57 - protein, 118 aa, Homo sapiens; identical to SEQ ID NO: 56

SEQ ID NO: 58 - protein, 118 aa, Homo sapiens
QVQLVESGGGVVQPGRSLRLSCAASGFTFSNYGIHWVRQAPGKGLEWVAVIWYDGSHKYYADSMKGRFTISRDNSKNTLDLQMNSLRAEDTAVYYCARGSSSWYFDLWGRGTLVTVSS

SEQ ID NO: 59 - protein, 121 aa, Homo sapiens
QVQLQESGPGLVKPSETLSLTCTVSGDSISSYYWSWIRQPPGKGLEWIGYIYYSGSTRYNPPLKSRVTISVDTSKNQFSLKLSSVTAADTAVYYCARICSSTSCWGWFDPWGQGTLVTVSS

SEQ ID NO: 60 - protein, 113 aa, Homo sapiens
QVQLVQSGAEVKKPGSSVKVSCKASGGTFSSYAISWVRQAPGQGLEWMGRIIPILGIVNYAQKFQGRVTITADKSTSTAYMELSSLRSEDTAVYYCARGKDIWGQGTMVTVSS

SEQ ID NO: 61 - protein, 113 aa, Homo sapiens
QVQLVQSGAEVKKPGSSVKVSCKASGGTFSSYAISWVRQAPGQGLEWMGRIIPILGIAQYAQKFQGRVTLTADKSTSTAYMELSSLRSEDTAVYYCARGKDIWGQGTMVTVSS

SEQ ID NO: 62 - protein, 113 aa, Homo sapiens
QVQLVQSGAEVKKPGSSVKVSCKASGGTFSSYAISWVRQAPGQGLEWMGRIIPILGIAQYAQKFQGRVTLTADKSTNTAYMELSSLRSEDTAVYYCARGKDIWGQGTMVTVSS

SEQ ID NO: 63 - protein, 113 aa, Homo sapiens
QVQLVQSGAEVKKPGSSVKVSCKASGGTFSSYAINWVRQAPGQGLEWMGRLIPILGIANYAQKFQGRVTITADKSTSTAYMELSSLRSEDTAVYYCARGKDIWGQGTMVTVSS

SEQ ID NO: 64 - protein, 107 aa, Homo sapiens; identical to SEQ ID NO: 36

SEQ ID NO: 65 - protein, 107 aa, Homo sapiens; identical to SEQ ID NO: 36

SEQ ID NO: 66 - protein, 107 aa, Homo sapiens; identical to SEQ ID NO: 36

SEQ ID NO: 67 - protein, 108 aa, Homo sapiens; identical to SEQ ID NO: 35

SEQ ID NO: 68 - protein, 108 aa, Homo sapiens
EIVLTQSPGTLSLSPGERATLSCRASQIVISTYLAWYQQKPGQAPRLLIYGASSRATGIPDRFSGSGSGTDFTLTISRLEPEDFAVYYCQQYGSSPCTFGQGTKLEIK

SEQ ID NO: 69 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTKLEIK

SEQ ID NO: 70 - protein, 108 aa, Homo sapiens
EIVLTQSPGTLSLSPGERATLSCRASQSVSNNYLAWYQQKPGQAPRLLIYGASSRATGIPDRFSGSGSGTDFTLTISRLEPEDFAVYYCQQYGSSPCTFGQGTKLEIK

SEQ ID NO: 71 - protein, 108 aa, Homo sapiens
EIVLTQSPGTLSLSPGERVTLSCRASQSVSSNYLAWYQQKPGQAPRLLIKGASSRATGIPDRFSGSGSGTDFTLTISRLEPEDFAVYYCQQYGSSPCTFGQGTKLEIK

SEQ ID NO: 72 - DNA, 354 bp, Homo sapiens
caggtgcaactggtggagtctgggggaggcgtggtccagcctgggaggtccctgagactctcctgtgcagcgtctggattcaccttccgtagctatggcatacactgggtccgccaggctccaggcaaggggctggagtgggtggcagttatatggtatgatggaagtcataaatactatgcagactccgtgaagggccgattcaccatctccagagacaattccaagaatacgctggatctgcaaatgaacagcctgagagccgaggacacggctgtgtattactgtgtgagaggaagcagcaactggtattttgactactggggccagggaaccctggtcaccgtctcctca

SEQ ID NO: 73 - DNA, 354 bp, Homo sapiens; identical to SEQ ID NO: 72

SEQ ID NO: 74 - DNA, 354 bp, Homo sapiens
caggtgcagttggtggagtctgggggaggcgtggtccagcctgggaggtccctgagactctcctgtgcagcgtctggattcaccttcagtaactatggcatacactgggtccgccaggctccaggcaaggggctggagtgggtggcagttatatggtatgatggaagtcataaatactatgcagactccatgaagggccgattcaccatctccagagacaattccaagaacacgctggatctacaaatgaacagcctgagagccgaggacacggctgtgtattactgtgcgagagggagcagcagctggtatttcgatctctggggccgtggcaccctggtcactgtctcctca

SEQ ID NO: 75 - DNA, 363 bp, Homo sapiens
caggtgcagctgcaggagtcgggcccaggactggtgaagccttcggagaccctgtccctcacctgcactgtctctggtgactccatcagtagttactactggagctggatccggcagcccccagggaagggactggagtggattggatatatctattacagtgggagcacccgctacaatccccccctcaagagtcgagtcaccatatcagtagacacgtccaagaaccagttctccctgaagctgagctctgtgaccgctgcggacacggccgtgtattactgtgcgaggatttgtagtagtaccagctgttggggctggttcgacccctggggccagggaaccctggtcaccgtctcctca

SEQ ID NO: 76 - DNA, 338 bp, Homo sapiens
caggtccagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagtaaactacgcacagaagttccagggcagagtcacgattaccgcggacaaatccacgagcacagcctacatggagctgagcagcctgagatctgaggacacggccgtgtattactgtgcgagagggaaggatatctggggccaagggacaatggtcaccgtctcttc

SEQ ID NO: 77 - DNA, 339 bp, Homo sapiens
caggtccagctggtgcagtctggggctgaggtgaagaagcctgggtcctcggtgaaggtctcctgtaaggcttctggaggcaccttcagcagctatgctatcagctgggtgcgacaggcccctggacaagggcttgagtggatgggaaggatcatccctatccttggtatagcacagtacgcacagaagttccagggcagagtcacgcttaccgcggacaaatccacgagcacagcctacatggagctgagcagcctgagatctgaggacacggccgtgtattactgtgcgagagggaaggatatctggggccaagggacaatggtcaccgtctcttca

SEQ ID NO: 78 - DNA, 339 bp, Homo sapiens
caggtccagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcacagtacgcacagaagttccagggcagagtcacgcttaccgcggacaaatccacgaacacagcctacatggaactgagcagcctgagatctgaggacacggccgtgtattactgtgcgagagggaaggatatctggggccaagggacaatggtcaccgtctcttca

SEQ ID NO: 79 - DNA, 339 bp, Homo sapiens
caggtccagctggtgcagtctggggctgaggtgaagaagcctgggtcctcggtgaaggtctcctgcaaggcttctggaggcaccttcagcagctatgctatcaactgggtgcgacaggcccctggacaagggcttgagtggatgggaaggctcatccctatccttggtatagcaaactacgcacagaagttccagggcagagtcacgattaccgcggacaaatccacgagcacagcctacatggagctgagcagcctgagatctgaggacacggccgtgtattactgtgcgagagggaaggatatctggggccaagggacaatggtcaccgtctcttca

SEQ ID NO: 80 - DNA, 321 bp, Homo sapiens
gacatccagatgacccagtctccatcctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttaccctctcactttcggcggagggaccaaggtggagatcaaa

SEQ ID NO: 81 - DNA, 321 bp, Homo sapiens; identical to SEQ ID NO: 80

SEQ ID NO: 82 - DNA, 321 bp, Homo sapiens; identical to SEQ ID NO: 42

SEQ ID NO: 83 - DNA, 324 bp, Homo sapiens; identical to SEQ ID NO: 41

SEQ ID NO: 84 - DNA, 324 bp, Homo sapiens
gaaattgtgttgacgcagtctccaggcaccctgtctttgtctccaggggaaagagccaccctctcctgcagggccagtcagattgttatcagcacctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagttcaccgtgcacttttggccaggggaccaagctggagatcaaa

SEQ ID NO: 85 - DNA, 324 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagccagaagattttgcagtgtattactgtcagcagtatggtagctcaccgtgcacttttggccaggggaccaagctggagatcaaa

SEQ ID NO: 86 - DNA, 324 bp, Homo sapiens
gaaattgtgttgacgcagtctccaggcaccctgtctttgtctccaggggaaagagccaccctctcctgcagggccagtcagagtgttagcaacaactacttagcctggtaccagcagaaacctggccaggctcccaggctcctcatctatggtgcatccagcagggccactggcatcccagacaggttcagtggcagtgggtctgggacagacttcactctcaccatcagcagactggagccagaagattttgcagtgtattactgtcagcagtatggtagctcaccgtgcacttttggccaggggaccaagctggagatcaaa

SEQ ID NO: 87 - DNA, 324 bp, Homo sapiens
gaaattgtgttgacgcagtctccaggcaccctgtctttgtctccaggggaaagagtcaccctctcctgcagggccagtcagagtgttagcagcaactacttagcctggtaccagcagaaacctggccaggctcccaggctcctcatcaaaggtgcatctagcagggccactggcatcccagacaggttcagtggcagtgggtctgggacagacttcactctcaccatcagcagactggagcctgaagattttgcagtgtattactgtcagcagtatggtagctcaccgtgcacttttggccaggggaccaagctggagatcaaa

SEQ ID NO: 88 - protein, 15 aa, Homo sapiens
YFDYWGQGTLVTVSS

SEQ ID NO: 89 - protein, 16 aa, Homo sapiens
WYFDLWGRGTLVTVSS

SEQ ID NO: 90 - protein, 13 aa, Homo sapiens
DIWGQGTMVTVSS

SEQ ID NO: 91 - protein, 98 aa, Homo sapiens
QVQLVQSGAEVKKPGSSVKVSCKASGGTFSSYTISWVRQAPGQGLEWMGRIIPILGIANYAQKFQGRVTITADKSTSTAYMELSSLRSEDTAVYYCAR

SEQ ID NO: 92 - protein, 15 aa, Homo sapiens
ISMLYLDENEKVVLK

SEQ ID NO: 93 - protein, 17 aa, Homo sapiens
QAKHKQRKRLKSSCKRH